(19) United States
(12) Patent Application Publication
(10) Pub. No.: US 2025/0075209 A1
(43) Pub. Date: Mar. 6, 2025

(54) RNAS FOR COMPLEMENT INHIBITION

(71) Applicant: Apellis Pharmaceuticals, Inc., Waltham, MA (US)

(72) Inventor: Jasmin Gegner, Schweinfurt (DE)

(21) Appl. No.: 18/724,567

(22) PCT Filed: Dec. 23, 2022
(86) PCT No.: PCT/US22/53920

§ 371 Date: Jun. 26, 2024

Related U.S. Application Data
(60) Provisional application No. 63/293,961, filed on Dec. 27, 2021.

(51) Int. Cl. C12N 15/113; A61K 38/12; A61P 37/06; C07K 16/18

(52) U.S. Cl. CPC C12N 15/113; A61K 38/12; A61P 37/06; C07K 16/18; C12N 2310/11; C12N 2310/14; C12N 2310/315; C12N 2310/321; C12N 2310/322; C12N 2310/351

(57) ABSTRACT

RNAs, such as miRNA and siRNA, and their use in treating complement-mediated disorders, are described.

### CROSS REFERENCE TO RELATED APPLICATIONS

This application claims the benefit of U.S. Provisional Application No. 63/293,961, filed Dec. 27, 2021, the contents of which is hereby incorporated by reference in its entirety.

### BACKGROUND

Complement is a system consisting of more than 30 plasma and cell-bound proteins that plays a significant role in both innate and adaptive immunity. The proteins of the complement system act in a series of enzymatic cascades through a variety of protein interactions and cleavage events. Complement activation occurs via three main pathways: the antibody-dependent classical pathway, the alternative pathway, and the mannose-binding lectin (MBL) pathway. Inappropriate or excessive complement activation is an underlying cause or contributing factor to a number of serious diseases and conditions, and considerable effort has been devoted over the past several decades to exploring various complement inhibitors as therapeutic agents.

### SUMMARY

In one aspect, the disclosure features an siRNA comprising an antisense strand and a sense strand, wherein the antisense strand is complementary to a nucleotide sequence that is at least 90% identical to any one of SEQ ID NOs: 76-99.

In some embodiments, the antisense strand is complementary to a nucleotide sequence comprising a sequence that differs by no more than 1, 2, 3, or 4 nucleotides from any one of SEQ ID NOs: 76-99. In some embodiments, the antisense strand is complementary to a nucleotide sequence comprising any one of SEQ ID NOs: 76-99. In some embodiments, the antisense strand comprises a nucleotide sequence comprising any one of SEQ ID NOs: 100-123.

In some embodiments, one or both of the sense strand and the antisense strand comprises at least one overhang region. In some embodiments, the at least one overhang comprises a 1, 2, 3, 4, or 5, nucleotide overhang. In some embodiments, the at least one overhang comprises a 3′ overhang. In some embodiments, the overhang region is complementary to a fragment of SEQ ID NO: 75. In some embodiments, the 3′ overhang of the siRNA comprises a 2-nucleotide overhang.

In some embodiments, an siRNA comprises a sense strand and an antisense strand that comprises at least one additional nucleotide on the 5′ end, the 3′ end, or both the 5′ end and the 3′ end, which is not complementary to a fragment of SEQ ID NO: 75.

In some embodiments, one or both of the sense stand and the antisense strand of the siRNA comprises at least one modified nucleotide. In some embodiments, the at least one modified nucleotide comprises a nucleotide that includes a 2′-O-Methyl group, a nucleotide that includes a 2′-Fluoro group, and/or a phosphorothioate bond with an adjacent nucleotide.

In some embodiments, the sense strand of the siRNA comprises the nucleotide sequence of any one of SEQ ID NOs: 76-99, 124-147, 220, 222, 224, 226, 228, 230, 232, 234, 236, 238, 240, 242, 244, 246, 248, 250, 252, 254, 256, 258, 260, 262, 264, and 266. In some embodiments, the antisense strand of the siRNA comprises the nucleotide sequence of any one of SEQ ID NOs: 100-123, 148-219, 221, 223, 225, 227, 229, 231, 233, 235, 237, 239, 241, 243, 245, 247, 249, 251, 253, 255, 257, 259, 261, 263, 265, and 267.

In some embodiments, the siRNA comprises a sense strand nucleotide sequence/antisense strand nucleotide sequence of any one of the following sets of sense/antisense SEQ ID NOs: 220/221, 222/223, 224/225, 226/227, 228/229, 230/231, 232/233, 234/235, 236/237, 238/239, 240/241, 242/243, 244/245, 246/247, 248/249, 250/251, 252/253, 254/255, 256/257, 258/259, 260/261, 262/263, 264/265, and 266/267.

In some embodiments, the siRNA comprises at least one ligand attached to one or all of the 5′ end of the sense strand, the 3′ end of the sense strand, the 5′ end of the antisense strand, and the 3′ end of the antisense strand. In some embodiments, the ligand comprises at least one GalNAc moiety. In some embodiments, the ligand comprises three GalNAc moieties.

In another aspect, the disclosure features a method of treating a subject having or at risk of a complement-mediated disorder, the method comprising administering to the subject a composition comprising an effective amount of the siRNA. In some embodiments, the method comprises administering to the subject a composition comprising a nucleic acid encoding the siRNA. In some embodiments, the subject is a human.

In some embodiments, after the administration of the composition, a level of factor B transcript or factor B protein in the subject or in a biological sample (e.g., a blood, serum or plasma sample, and/or a sample comprising hepatocytes) from the subject is reduced relative to a level before the administration of the composition. In some embodiment, the level of factor B transcript or factor B protein is reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90%, relative to a level before the administration.

In some embodiments, the composition is administered intravenously or subcutaneously to the subject. In some embodiments, the composition is administered to a hepatocyte of the subject. In some embodiments, the composition is administered to the hepatocyte ex vivo. In some embodiments, the composition is administered to the hepatocyte in vivo.

In some embodiments, the methods include administering to the subject a second agent. In some embodiments, the second agent is an anti-factor B antibody or a compstatin analog.

In some embodiments, the subject has a defect in complement regulation, optionally wherein the defect comprises abnormally low expression of one or more complement regulatory proteins by at least some of the subject's cells. In some embodiments, the complement-mediated disorder is a chronic disorder. In some embodiments, the complement-mediated disorder involves complement-mediated damage to red blood cells, optionally wherein the disorder is paroxysmal nocturnal hemoglobinuria or atypical hemolytic uremic syndrome. In some embodiments, the complement-mediated disorder is an autoimmune disease, optionally wherein the disorder is multiple sclerosis. In some embodiments, the complement-mediated disorder involves the kidney, optionally wherein the disorder is membranoproliferative glomerulonephritis, lupus nephritis, IgA nephropathy (IgAN), primary membranous nephropathy (primary MN), C3 glomerulopathy (C3G), or acute kidney injury. In some embodiments, the complement-mediated disorder involves the central or peripheral nervous system or neuromuscular junction, optionally wherein the disorder is neuromyelitis optica, Guillain-Barré syndrome, multifocal motor neuropathy, or myasthenia gravis.

In some embodiments, the composition comprises a carrier and/or excipient.

In another aspect, the disclosure features an expression vector comprising one or more nucleotide sequences encoding one or more of the siRNAs described herein. In some embodiments, the expression vector comprises a nucleotide sequence encoding a factor B inhibitor (e.g., an aptamer, an anti-factor B antibody, a mammalian complement regulatory protein, or mini factor H).

In another aspect, the disclosure features an antisense nucleic acid comprising the nucleotide sequence of any one of SEQ ID NOs: 100-123, 148-219, 221, 223, 225, 227, 229, 231, 233, 235, 237, 239, 241, 243, 245, 247, 249, 251, 253, 255, 257, 259, 261, 263, 265, and 267.

In another aspect, the disclosure features a method of reducing or inhibiting complement factor B expression in a cell. In some embodiments, the method comprises contacting the cell with an siRNA comprising an antisense strand and a sense strand, wherein the antisense strand is complementary to a nucleotide sequence that is at least 90% identical to any one of SEQ ID NOs: 76-99. In some embodiments, the antisense strand is complementary to a nucleotide sequence comprising a sequence that differs by no more than 1, 2, 3, or 4 nucleotides from any one of SEQ ID NOs: 76-99. In some embodiments, the antisense strand is complementary to a nucleotide sequence comprising any one of SEQ ID NOs: 76-99. In some embodiments, the antisense strand comprises a nucleotide sequence comprising any one of SEQ ID NOs: 100-123. In some embodiments, one or both of the sense strand and the antisense strand comprises at least one overhang region. In some embodiments, the at least one overhang comprises a 1, 2, 3, 4, or 5, nucleotide overhang. In some embodiments, the at least one overhang comprises a 3′ overhang. In some embodiments, the overhang region is complementary to a fragment of SEQ ID NO: 75. In some embodiments, the 3′ overhang of the siRNA comprises a 2-nucleotide overhang. In some embodiments, an siRNA comprises a sense strand and an antisense strand that comprises at least one additional nucleotide on the 5′ end, the 3′ end, or both the 5′ end and the 3′ end, which is not complementary to a fragment of SEQ ID NO: 75. In some embodiments, one or both of the sense stand and the antisense strand of the siRNA comprises at least one modified nucleotide. In some embodiments, the at least one modified nucleotide comprises a nucleotide that includes a 2′-O-Methyl group, a nucleotide that includes a 2′-Fluoro group, and/or a phosphorothioate bond with an adjacent nucleotide. In some embodiments, the sense strand of the siRNA comprises the nucleotide sequence of any one of SEQ ID NOs: 76-99, 124-147, 220, 222, 224, 226, 228, 230, 232, 234, 236, 238, 240, 242, 244, 246, 248, 250, 252, 254, 256, 258, 260, 262, 264, and 266. In some embodiments, the antisense strand of the siRNA comprises the nucleotide sequence of any one of SEQ ID NOs: 100-123, 148-219, 221, 223, 225, 227, 229, 231, 233, 235, 237, 239, 241, 243, 245, 247, 249, 251, 253, 255, 257, 259, 261, 263, 265, and 267. In some embodiments, the siRNA comprises a sense strand nucleotide sequence/antisense strand nucleotide sequence of any one of the following sets of sense/antisense SEQ ID NOs: 220/221, 222/223, 224/225, 226/227, 228/229, 230/231, 232/233, 234/235, 236/237, 238/239, 240/241, 242/243, 244/245, 246/247, 248/249, 250/251, 252/253, 254/255, 256/257, 258/259, 260/261, 262/263, 264/265, and 266/267. In some embodiments, the siRNA comprises at least one ligand attached to one or all of the 5′ end of the sense strand, the 3′ end of the sense strand, the 5′ end of the antisense strand, and the 3′ end of the antisense strand. In some embodiments, the ligand comprises at least one GalNAc moiety. In some embodiments, the ligand comprises three GalNAc moieties.

In some embodiments, the method comprises contacting the cell with an antisense nucleic acid comprising the nucleotide sequence of any one of SEQ ID NOs: 100-123, 148-219, 221, 223, 225, 227, 229, 231, 233, 235, 237, 239, 241, 243, 245, 247, 249, 251, 253, 255, 257, 259, 261, 263, 265, and 267.

In some embodiments, the method comprises contacting the cell with a composition or an expression vector described herein.

In some embodiments, after the contacting step, the level of factor B transcript or factor B protein is reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90%, relative to a level before the contacting step. In some embodiments, the method comprises maintaining the cell for a time sufficient to obtain degradation of the mRNA transcript of a complement factor B gene, thereby inhibiting expression of the complement factor B gene in the cell.

In some embodiments, the cell is in a subject. In some embodiments, the subject is a human. In some embodiments, the subject suffers from a complement-mediated disorder.

In another aspect, the disclosure features a method of reducing or inhibiting expression of factor B in a subject, the method comprising contacting a cell of the subject with an siRNA comprising an antisense strand and a sense strand, wherein the antisense strand is complementary to a nucleotide sequence that is at least 90% identical to any one of SEQ ID NOs: 76-99. In some embodiments, the antisense strand is complementary to a nucleotide sequence comprising a sequence that differs by no more than 1, 2, 3, or 4 nucleotides from any one of SEQ ID NOs: 76-99. In some embodiments, the antisense strand is complementary to a nucleotide sequence comprising any one of SEQ ID NOs: 76-99. In some embodiments, the antisense strand comprises a nucleotide sequence comprising any one of SEQ ID NOs: 100-123. In some embodiments, one or both of the sense strand and the antisense strand comprises at least one overhang region. In some embodiments, the at least one overhang comprises a 1, 2, 3, 4, or 5, nucleotide overhang. In some embodiments, the at least one overhang comprises a 3′ overhang. In some embodiments, the overhang region is complementary to a fragment of SEQ ID NO: 75. In some embodiments, the 3′ overhang of the siRNA comprises a 2-nucleotide overhang. In some embodiments, an siRNA comprises a sense strand and an antisense strand that comprises at least one additional nucleotide on the 5′ end, the 3′ end, or both the 5′ end and the 3′ end, which is not complementary to a fragment of SEQ ID NO: 75. In some embodiments, one or both of the sense stand and the antisense strand of the siRNA comprises at least one modified nucleotide. In some embodiments, the at least one modified nucleotide comprises a nucleotide that includes a 2′-O-Methyl group, a nucleotide that includes a 2′-Fluoro group, and/or a phosphorothioate bond with an adjacent nucleotide. In some embodiments, the sense strand of the siRNA comprises the nucleotide sequence of any one of SEQ ID NOs: 76-99, 124-147, 220, 222, 224, 226, 228, 230, 232, 234, 236, 238, 240, 242, 244, 246, 248, 250, 252, 254, 256, 258, 260, 262, 264, and 266. In some embodiments, the antisense strand of the siRNA comprises the nucleotide sequence of any one of SEQ ID NOs: 100-123, 148-219, 221, 223, 225, 227, 229, 231, 233, 235, 237, 239, 241, 243, 245, 247, 249, 251, 253, 255, 257, 259, 261, 263, 265, and 267. In some embodiments, the siRNA comprises a sense strand nucleotide sequence/antisense strand nucleotide sequence of any one of the following sets of sense/antisense SEQ ID NOs: 220/221, 222/223, 224/225, 226/227, 228/229, 230/231, 232/233, 234/235, 236/237, 238/239, 240/241, 242/243, 244/245, 246/247, 248/249, 250/251, 252/253, 254/255, 256/257, 258/259, 260/261, 262/263, 264/265, and 266/267. In some embodiments, the siRNA comprises at least one ligand attached to one or all of the 5′ end of the sense strand, the 3′ end of the sense strand, the 5′ end of the antisense strand, and the 3′ end of the antisense strand. In some embodiments, the ligand comprises at least one GalNAc moiety. In some embodiments, the ligand comprises three GalNAc moieties.

In some embodiments, the method comprises contacting the cell with an antisense nucleic acid comprising the nucleotide sequence of any one of SEQ ID NOs: 100-123, 148-219, 221, 223, 225, 227, 229, 231, 233, 235, 237, 239, 241, 243, 245, 247, 249, 251, 253, 255, 257, 259, 261, 263, 265, and 267.

In some embodiments, the method comprises contacting the cell with a composition or an expression vector described herein.

In some embodiments, after the contacting step, the level of factor B transcript or factor B protein is reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90%, relative to a level before the contacting step.

In some embodiments, the subject is a human. In some embodiments, the subject suffers from a complement-mediated disorder.

In another aspect, the disclosure features a method of reducing or inhibiting expression of factor B in a subject, the method comprising administering to the subject an siRNA comprising an antisense strand and a sense strand, wherein the antisense strand is complementary to a nucleotide sequence that is at least 90% identical to any one of SEQ ID NOs: 76-99. In some embodiments, the antisense strand is complementary to a nucleotide sequence comprising a sequence that differs by no more than 1, 2, 3, or 4 nucleotides from any one of SEQ ID NOs: 76-99. In some embodiments, the antisense strand is complementary to a nucleotide sequence comprising any one of SEQ ID NOs: 76-99. In some embodiments, the antisense strand comprises a nucleotide sequence comprising any one of SEQ ID NOs: 100-123. In some embodiments, one or both of the sense strand and the antisense strand comprises at least one overhang region. In some embodiments, the at least one overhang comprises a 1, 2, 3, 4, or 5, nucleotide overhang. In some embodiments, the at least one overhang comprises a 3′ overhang. In some embodiments, the overhang region is complementary to a fragment of SEQ ID NO: 75. In some embodiments, the 3′ overhang of the siRNA comprises a 2-nucleotide overhang. In some embodiments, an siRNA comprises a sense strand and an antisense strand that comprises at least one additional nucleotide on the 5′ end, the 3′ end, or both the 5′ end and the 3′ end, which is not complementary to a fragment of SEQ ID NO: 75. In some embodiments, one or both of the sense stand and the antisense strand of the siRNA comprises at least one modified nucleotide. In some embodiments, the at least one modified nucleotide comprises a nucleotide that includes a 2′-O-Methyl group, a nucleotide that includes a 2′-Fluoro group, and/or a phosphorothioate bond with an adjacent nucleotide. In some embodiments, the sense strand of the siRNA comprises the nucleotide sequence of any one of SEQ ID NOs: 76-99, 124-147, 220, 222, 224, 226, 228, 230, 232, 234, 236, 238, 240, 242, 244, 246, 248, 250, 252, 254, 256, 258, 260, 262, 264, and 266. In some embodiments, the antisense strand of the siRNA comprises the nucleotide sequence of any one of SEQ ID NOs: 100-123, 148-219, 221, 223, 225, 227, 229, 231, 233, 235, 237, 239, 241, 243, 245, 247, 249, 251, 253, 255, 257, 259, 261, 263, 265, and 267. In some embodiments, the siRNA comprises a sense strand nucleotide sequence/antisense strand nucleotide sequence of any one of the following sets of sense/antisense SEQ ID NOs: 220/221, 222/223, 224/225, 226/227, 228/229, 230/231, 232/233, 234/235, 236/237, 238/239, 240/241, 242/243, 244/245, 246/247, 248/249, 250/251, 252/253, 254/255, 256/257, 258/259, 260/261, 262/263, 264/265, and 266/267. In some embodiments, the siRNA comprises at least one ligand attached to one or all of the 5′ end of the sense strand, the 3′ end of the sense strand, the 5′ end of the antisense strand, and the 3′ end of the antisense strand. In some embodiments, the ligand comprises at least one GalNAc moiety. In some embodiments, the ligand comprises three GalNAc moieties.

In some embodiments, the method comprises administering an antisense nucleic acid comprising the nucleotide sequence of any one of SEQ ID NOs: 100-123, 148-219, 221, 223, 225, 227, 229, 231, 233, 235, 237, 239, 241, 243, 245, 247, 249, 251, 253, 255, 257, 259, 261, 263, 265, and 267.

In some embodiments, the method comprises administering a composition or an expression vector described herein.

In some embodiments, after the administering step, the level of factor B transcript or factor B protein is reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90%, relative to a level before the administering step.

In some embodiments, the subject is a human. In some embodiments, the subject suffers from a complement-mediated disorder.

In another aspect, the disclosure features a method of reducing or inhibiting complement in a subject, the method comprising administering to the subject an siRNA comprising an antisense strand and a sense strand, wherein the antisense strand is complementary to a nucleotide sequence that is at least 90% identical to any one of SEQ ID NOs: 76-99. In some embodiments, the antisense strand is complementary to a nucleotide sequence comprising a sequence that differs by no more than 1, 2, 3, or 4 nucleotides from any one of SEQ ID NOs: 76-99. In some embodiments, the antisense strand is complementary to a nucleotide sequence comprising any one of SEQ ID NOs: 76-99. In some embodiments, the antisense strand comprises a nucleotide sequence comprising any one of SEQ ID NOs: 100-123. In some embodiments, one or both of the sense strand and the antisense strand comprises at least one overhang region. In some embodiments, the at least one overhang comprises a 1, 2, 3, 4, or 5, nucleotide overhang. In some embodiments, the at least one overhang comprises a 3′ overhang. In some embodiments, the overhang region is complementary to a fragment of SEQ ID NO: 75. In some embodiments, the 3′ overhang of the siRNA comprises a 2-nucleotide overhang. In some embodiments, an siRNA comprises a sense strand and an antisense strand that comprises at least one additional nucleotide on the 5′ end, the 3′ end, or both the 5′ end and the 3′ end, which is not complementary to a fragment of SEQ ID NO: 75. In some embodiments, one or both of the sense stand and the antisense strand of the siRNA comprises at least one modified nucleotide. In some embodiments, the at least one modified nucleotide comprises a nucleotide that includes a 2′-O-Methyl group, a nucleotide that includes a 2′-Fluoro group, and/or a phosphorothioate bond with an adjacent nucleotide. In some embodiments, the sense strand of the siRNA comprises the nucleotide sequence of any one of SEQ ID NOs: 76-99, 124-147, 220, 222, 224, 226, 228, 230, 232, 234, 236, 238, 240, 242, 244, 246, 248, 250, 252, 254, 256, 258, 260, 262, 264, and 266. In some embodiments, the antisense strand of the siRNA comprises the nucleotide sequence of any one of SEQ ID NOs: 100-123, 148-219, 221, 223, 225, 227, 229, 231, 233, 235, 237, 239, 241, 243, 245, 247, 249, 251, 253, 255, 257, 259, 261, 263, 265, and 267. In some embodiments, the siRNA comprises a sense strand nucleotide sequence/antisense strand nucleotide sequence of any one of the following sets of sense/antisense SEQ ID NOs: 220/221, 222/223, 224/225, 226/227, 228/229, 230/231, 232/233, 234/235, 236/237, 238/239, 240/241, 242/243, 244/245, 246/247, 248/249, 250/251, 252/253, 254/255, 256/257, 258/259, 260/261, 262/263, 264/265, and 266/267. In some embodiments, the siRNA comprises at least one ligand attached to one or all of the 5′ end of the sense strand, the 3′ end of the sense strand, the 5′ end of the antisense strand, and the 3′ end of the antisense strand. In some embodiments, the ligand comprises at least one GalNAc moiety. In some embodiments, the ligand comprises three GalNAc moieties.

In some embodiments, the method comprises administering an antisense nucleic acid comprising the nucleotide sequence of any one of SEQ ID NOs: 100-123, 148-219, 221, 223, 225, 227, 229, 231, 233, 235, 237, 239, 241, 243, 245, 247, 249, 251, 253, 255, 257, 259, 261, 263, 265, and 267.

In some embodiments, the method comprises administering a composition or an expression vector described herein.

In some embodiments, after the administering step, complement activity is reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90%, relative to a control, e.g., a control level of complement activity before the administering step.

In some embodiments, the subject is a human. In some embodiments, the subject suffers from a complement-mediated disorder.

### Definitions

Antibody: As used herein, the term “antibody” refers to an immunoglobulin or a derivative thereof containing an immunoglobulin domain capable of binding to an antigen. The antibody can be of any species, e.g., human, rodent, rabbit, goat, chicken, etc. The antibody may be a member of any immunoglobulin class, including any of the human classes: IgG, IgM, IgA, IgD, and IgE, or subclasses thereof such as IgG1, IgG2, etc. In various embodiments of the invention the antibody is a fragment such as an Fab′, F(ab′)₂, scFv (single-chain variable) or other fragment that retains an antigen binding site, or a recombinantly produced scFv fragment, including recombinantly produced fragments. See, e.g., Allen, T., Nature Reviews Cancer, Vol. 2, 750-765, 2002, and references therein. The antibody can be monovalent, bivalent or multivalent. The antibody may be a chimeric or “humanized” antibody in which, for example, a variable domain of rodent origin is fused to a constant domain of human origin, thus retaining the specificity of the rodent antibody. The domain of human origin need not originate directly from a human in the sense that it is first synthesized in a human being. Instead, “human” domains may be generated in rodents whose genome incorporates human immunoglobulin genes. See, e.g., Vaughan, et al., (1998), Nature Biotechnology, 16:535-539. The antibody may be partially or completely humanized. An antibody may be polyclonal or monoclonal, though for purposes of the present invention monoclonal antibodies are generally preferred. Methods for producing antibodies that specifically bind to virtually any molecule of interest are known in the art. For example, monoclonal or polyclonal antibodies can be purified from blood or ascites fluid of an animal that produces the antibody (e.g., following natural exposure to or immunization with the molecule or an antigenic fragment thereof), can be produced using recombinant techniques in cell culture or transgenic organisms, or can be made at least in part by chemical synthesis.

Approximately: As used herein, the terms “approximately” or “about” in reference to a number are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value).

Complementary: As used herein, in accordance with its art-accepted meaning, “complementary” refers to the capacity for precise pairing between particular bases, nucleosides, nucleotides or nucleic acids. For example, adenine (A) and uridine (U) are complementary; adenine (A) and thymidine (T) are complementary; and guanine (G) and cytosine (C), are complementary and are referred to in the art as Watson-Crick base pairings. If a nucleotide at a certain position of a first nucleic acid sequence is complementary to a nucleotide located opposite in a second nucleic acid sequence when the strands are aligned in anti-parallel orientation, the nucleotides form a complementary base pair, and the nucleic acids are complementary at that position. The percent complementarity of a first nucleic acid to a second nucleic acid may be evaluated by aligning them in antiparallel orientation for maximum complementarity over a window of evaluation, determining the total number of nt in both strands that form complementary base pairs within the window, dividing by the total number of nt within the window, and multiplying by 100. For example, AAAAAAAA and TTTGTTAT are 75% complementary since there are 12 nt in complementary base pairs out of a total of 16 nt. When computing the number of complementary nt needed to achieve a particular % complementarity, fractions are rounded to the nearest whole number. A position occupied by non-complementary nucleotides constitutes a mismatch, i.e., the position is occupied by a non-complementary base pair. In certain embodiments a window of evaluation has the length described herein for duplex portions or target portions. Complementary sequences include base-pairing of a polynucleotide comprising a first nucleotide sequence to a polynucleotide comprising a second nucleotide sequence over the entire length of both nucleotide sequences (if the same length) or over the entire length of the shorter sequence (if different lengths). Such sequences can be referred to as “perfectly complementary” (100% complementarity) with respect to each other herein. Nucleic acids that are at least 70% complementary over a window of evaluation are considered “substantially complementary” over that window. In certain embodiments complementary nucleic acids are at least 75%, at least 80%, at least 85%, at least 90%, or at least 95% complementary over the window of evaluation. Where a first sequence is referred to as “substantially complementary” with respect to a second sequence herein, the two sequences may be perfectly complementary or they may comprise one or more unmatched bases upon hybridization, e.g., up to about 5%, 10%, 15%, 20%, or 25% unmatched bases upon hybridization, e.g., 1, 2, 3, 4, 5, or 6 mismatched base pairs upon hybridization for a duplex up to 30 base pairs, while retaining the ability to hybridize under the conditions most relevant to their intended use. It should be understood that where two oligonucleotides are designed to form, upon hybridization, one or more single stranded overhangs, such overhangs are not regarded as mismatches or unpaired nucleotides with regard to the determination of percent complementarity. For example, the two strands of a dsRNA comprising one oligonucleotide 21 nucleotides in length and another oligonucleotide 23 nucleotides in length, wherein the longer oligonucleotide comprises a sequence of 21 nucleotides that is perfectly complementary to the shorter oligonucleotide and a 2 nucleotide overhang, may be referred to as “perfectly complementary” herein. “Complementary” sequences, as used herein may include one or more non-Watson-Crick base pairs and/or base pairs formed from non-natural and other modified nucleotides, in so far as the requirements with respect to their ability to hybridize are fulfilled. Such non-Watson-Crick base pairs include, but are not limited to, G: U Wobble or Hoogsteen base pairing. Those of ordinary skill in the art are aware that guanine, cytosine, adenine, and uracil can be replaced by other bases without substantially altering the base pairing properties of a polynucleotide comprising a nucleotide bearing such bases, according to the so-called “wobble” rules (see, e.g., Murphy, FV IV & V Ramakrishnan, V., Nature Structural and Molecular Biology 11:1251-1252 (2004)). For example, a nucleotide comprising inosine as its base can base pair with nucleotides containing adenine, cytosine, or uracil. Thus, nucleotides containing uracil, guanine, or adenine can be replaced in the nucleotide sequences of an Inhibitory RNA described herein by a nucleotide containing, for example, inosine. It will be understood that the terms “complementary”, “perfectly complementary”, and “substantially complementary” can be used with respect to the base matching between any two nucleic acids, e.g., the base matching between the sense strand and the antisense strand of a dsRNA, or between the antisense strand of a ds inhibitory RNA (e.g., an siRNA) and a target sequence, or between an antisense oligonucleotide and a target sequence, as will be evident from the context. “Hybridize”, as used herein, refers to the interaction between two nucleic acid sequences comprising or consisting of complementary portions such that a duplex structure is formed that is stable under the particular conditions of interest, as will be understood by the ordinary skilled artisan.

Complement component: As used herein, the terms “complement component” or “complement protein” is a molecule that is involved in activation of the complement system or participates in one or more complement-mediated activities. Components of the classical complement pathway include, e.g., C1q, C1r, C1s, C2, C3, C4, C5, C6, C7, C8, C9, and the C5b-9 complex, also referred to as the membrane attack complex (MAC) and active fragments or enzymatic cleavage products of any of the foregoing (e.g., C3a, C3b, C4a, C4b, C5a, etc.). Components of the alternative pathway include, e.g., factors B, D, H, and I, and properdin, with factor H being a negative regulator of the pathway. Components of the lectin pathway include, e.g., MBL2, MASP-1, and MASP-2. Complement components also include cell-bound receptors for soluble complement components. Such receptors include, e.g., C5a receptor (C5aR), C3a receptor (C3aR), Complement Receptor 1 (CR1), Complement Receptor 2 (CR2), Complement Receptor 3 (CR3), etc. It will be appreciated that the term “complement component” is not intended to include those molecules and molecular structures that serve as “triggers” for complement activation, e.g., antigen-antibody complexes, foreign structures found on microbial or artificial surfaces, etc.

Host cell: As used herein, the term “host cell” refers to a cell into which exogenous DNA (recombinant or otherwise) has been introduced. Persons of skill upon reading this disclosure will understand that such terms refer not only to the particular subject cell, but also to the progeny of such a cell. Because certain modifications may occur in succeeding generations due to either mutation or environmental influences, such progeny may not, in fact, be identical to the parent cell, but are still included within the scope of the term “host cell” as used herein. In some embodiments, host cells include prokaryotic and eukaryotic cells selected from any of the Kingdoms of life that are suitable for expressing an exogenous DNA (e.g., a recombinant nucleic acid sequence). Exemplary cells include those of prokaryotes and eukaryotes (single-cell or multiple-cell), bacterial cells (e.g., strains of E. coli, Bacillus spp., Streptomyces spp., etc.), mycobacteria cells, fungal cells, yeast cells (e.g., S. cerevisiae, S. pombe, P. pastoris, P. methanolica, etc.), plant cells, insect cells (e.g., SF-9, SF-21, baculovirus-infected insect cells, Trichoplusia ni, etc.), non-human animal cells, human cells, or cell fusions such as, for example, hybridomas or quadromas. In some embodiments, the cell is a human, monkey, ape, hamster, rat, or mouse cell. In some embodiments, the cell is eukaryotic and is selected from the following cells: CHO (e.g., CHO K1, DXB-1 1 CHO, Veggie-CHO), COS (e.g., COS-7), retinal cell, Vero, CVI, kidney (e.g., HEK293, 293 EBNA, MSR 293, MDCK, HaK, BHK), HeLa, HepG2, WI38, MRC 5, Colo205, HB 8065, HL-60, (e.g., BHK21), Jurkat, Daudi, A431 (epidermal), CV-1, U937, 3T3, L cell, C127 cell, SP2/0, NS-0, MMT 060562, Sertoli cell, BRL 3 A cell, HT1080 cell, myeloma cell, tumor cell, and a cell line derived from an aforementioned cell. In some embodiments, the cell comprises one or more viral genes.

Identity: As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4:11-17), which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

Linked: As used herein, the term “linked”, when used with respect to two or more moieties, means that the moieties are physically associated or connected with one another to form a molecular structure that is sufficiently stable so that the moieties remain associated under the conditions in which the linkage is formed and, preferably, under the conditions in which the new molecular structure is used, e.g., physiological conditions. In certain preferred embodiments of the invention the linkage is a covalent linkage. In other embodiments the linkage is noncovalent. Moieties may be linked either directly or indirectly. When two moieties are directly linked, they are either covalently bonded to one another or are in sufficiently close proximity such that intermolecular forces between the two moieties maintain their association. When two moieties are indirectly linked, they are each linked either covalently or noncovalently to a third moiety, which maintains the association between the two moieties. In general, when two moieties are referred to as being linked by a “linker” or “linking moiety” or “linking portion”, the linkage between the two linked moieties is indirect, and typically each of the linked moieties is covalently bonded to the linker. The linker can be any suitable moiety that reacts with the two moieties to be linked within a reasonable period of time, under conditions consistent with stability of the moieties (which may be protected as appropriate, depending upon the conditions), and in sufficient amount, to produce a reasonable yield.

MicroRNA (miRNA): As used herein, the term “microRNA” or “miRNA” refers to a small non-coding RNA molecule that can function in transcriptional and/or post-transcriptional regulation of target gene expression. The terms encompass a mature miRNA sequence or a precursor miRNA sequence, including a primary transcript (pri-miRNA) and a stem-loop precursor (pre-miRNA). The biogenesis of a naturally occurring miRNA initiates in the nucleus by RNA polymerase II transcription, generating a primary transcript (pri-miRNA). The primary transcript is cleaved by Drosha ribonuclease III enzyme to produce an approximately 70 nt stem-loop precursor miRNA (pre-miRNA). The pre-miRNA is then actively exported to the cytoplasm where it is cleaved by Dicer ribonuclease to form the mature miRNA, which includes an “antisense strand” or “guide strand” (that includes a region that is substantially complementary to a target sequence) and a “sense strand” or “passenger strand” (that includes a region that is substantially complementary to a region of the antisense strand). Those of ordinary skill in the art will appreciate that a guide strand may be perfectly complementary to a target region of a target RNA or may have less than perfect complementarity to a target region of a target RNA. The guide strand of this miRNA is incorporated into an RNA-induced silencing complex (RISC) that recognizes target mRNAs through base pairing with the miRNA, and commonly results in translational inhibition or destabilization of the target mRNA. As is understood in the field, for naturally occurring miRNAs, target mRNA recognition occurs through imperfect base pairing with the mRNA. In some embodiments, an miRNA is synthetic or engineered, and target mRNA recognition occurs through perfect base pairing with the mRNA. Typically, the target mRNA contains a sequence complementary to a “seed” sequence of the miRNA, which usually corresponds to nucleotides 2-8 of the miRNA. Information concerning miRNAs and associated pri-miRNA and pre-miRNA sequences is available in miRNA databases such as miRBase (Griffiths-Jones et al. 2008 Nucl Acids Res 36, (Database Issue: D154-D158) and the NCBI human genome database.

Operably linked: As used herein, the term “operably linked” refers to a juxtaposition wherein the components described are in a relationship permitting them to function in their intended manner. A control element “operably linked” to a functional element is associated in such a way that expression and/or activity of the functional element is achieved under conditions compatible with the control element. In some embodiments, “operably linked” control elements are contiguous (e.g., covalently linked) with the coding elements of interest; in some embodiments, control elements act in trans to or otherwise at a from the functional element of interest.

Recombinant: As used herein, the term “recombinant” is intended to refer to polypeptides that are designed, engineered, prepared, expressed, created, manufactured, and/or or isolated by recombinant means, such as polypeptides expressed using a recombinant expression vector transfected into a host cell; polypeptides isolated from a recombinant, combinatorial human polypeptide library; polypeptides isolated from an animal (e.g., a mouse, rabbit, sheep, fish, etc.) that is transgenic for or otherwise has been manipulated to express a gene or genes, or gene components that encode and/or direct expression of the polypeptide or one or more component(s), portion(s), element(s), or domain(s) thereof; and/or polypeptides prepared, expressed, created or isolated by any other means that involves splicing or ligating selected nucleic acid sequence elements to one another, chemically synthesizing selected sequence elements, and/or otherwise generating a nucleic acid that encodes and/or directs expression of the polypeptide or one or more component(s), portion(s), element(s), or domain(s) thereof. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source such as, for example, in the germline of a source organism of interest (e.g., of a human, a mouse, etc.).

RNA interference: As used herein, the term “RNA interference” or “RNAi” refers generally to a process in which a double-stranded RNA molecule or a short hairpin RNA molecule reduces or inhibits expression of a nucleic acid sequence with which the double-stranded or short hairpin RNA molecule shares substantial or total homology. Without wishing to be bound by any theory, it is believed that, in nature, the RNAi pathway is initiated by a Type III endonuclease known as Dicer, which cleaves long double-stranded RNA (dsRNA) into double-stranded fragments typically of 21-23 base pairs with 2-base 3′ overhangs (although variations in length and overhangs are also contemplated), referred to as “short interfering RNAs” (“siRNAs”). Such siRNAs comprise two single-stranded RNAs (ssRNAs), with an “antisense strand” or “guide strand” that includes a region that is substantially complementary to a target sequence, and a “sense strand” or “passenger strand” that includes a region that is substantially complementary to a region of the antisense strand. Those of ordinary skill in the art will appreciate that a guide strand may be perfectly complementary to a target region of a target RNA or may have less than perfect complementarity to a target region of a target RNA.

Subject: As used herein, the term “subject” or “test subject” refers to any organism to which a provided compound or composition is administered in accordance with the present invention e.g., for experimental, diagnostic, prophylactic, and/or therapeutic purposes. Typical subjects include animals (e.g., mammals such as mice, rats, rabbits, non-human primates, and humans; insects; worms; etc.) and plants. In some embodiments, a subject may be suffering from, and/or susceptible to a disease, disorder, and/or condition.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and/or chemical phenomena.

Suffering from: An individual who is “suffering from” a disease, disorder, and/or condition has been diagnosed with and/or displays one or more symptoms of a disease, disorder, and/or condition.

Target gene: A “target gene”, as used herein, refers to a gene whose expression is to be modulated, e.g., inhibited. As used herein, the term “target RNA” refers to an RNA to be degraded or translationally repressed or otherwise inhibited using one or more miRNAs. A target RNA may also be referred to as a target sequence or target transcript. The RNA may be a primary RNA transcript transcribed from the target gene (e.g., a pre-mRNA) or a processed transcript, e.g., mRNA encoding a polypeptide. As used herein, the term “target portion” or “target region” refers to a contiguous portion of the nucleotide sequence of a target RNA. In some embodiments, a target portion an mRNA is at least long enough to serve as a substrate for RNA interference (RNAi)-mediated cleavage within that portion in the presence of a suitable inhibitory RNA. A target portion may be from about 8-36 nucleotides in length, e.g., about 10-20 or about 15-30 nucleotides in length. A target portion length may have specific value or subrange within the afore-mentioned ranges. For example, in certain embodiments a target portion may be between about 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 nucleotides in length.

Therapeutic agent: As used herein, the phrase “therapeutic agent” refers to any agent that, when administered to a subject, has a therapeutic effect and/or elicits a desired biological and/or pharmacological effect. In some embodiments, a therapeutic agent is any substance that can be used to alleviate, ameliorate, relieve, inhibit, prevent, delay onset of, reduce severity of, and/or reduce incidence of one or more symptoms or features of a disease, disorder, and/or condition.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or signs of the disease, disorder, and/or condition. In some embodiments, a therapeutically effective amount is administered in a single dose; in some embodiments, multiple unit doses are required to deliver a therapeutically effective amount.

Treating: As used herein, the term “treating” refers to providing treatment, i.e., providing any type of medical or surgical management of a subject. The treatment can be provided in order to reverse, alleviate, inhibit the progression of, prevent or reduce the likelihood of a disease, disorder, or condition, or in order to reverse, alleviate, inhibit or prevent the progression of, prevent or reduce the likelihood of one or more symptoms or manifestations of a disease, disorder or condition. “Prevent” refers to causing a disease, disorder, condition, or symptom or manifestation of such not to occur for at least a period of time in at least some individuals. Treating can include administering an agent to the subject following the development of one or more symptoms or manifestations indicative of a complement-mediated condition, e.g., in order to reverse, alleviate, reduce the severity of, and/or inhibit or prevent the progression of the condition and/or to reverse, alleviate, reduce the severity of, and/or inhibit or one or more symptoms or manifestations of the condition. A composition of the disclosure can be administered to a subject who has developed a complement-mediated disorder or is at increased risk of developing such a disorder relative to a member of the general population. A composition of the disclosure can be administered prophylactically, i.e., before development of any symptom or manifestation of the condition. Typically in this case the subject will be at risk of developing the condition.

Nucleic acid: The term “nucleic acid” includes any nucleotides, analogs thereof, and polymers thereof. The term “polynucleotide” as used herein refer to a polymeric form of nucleotides of any length, either ribonucleotides (RNA) or deoxyribonucleotides (DNA). These terms refer to the primary structure of the molecules and, thus, include double- and single-stranded DNA, and double- and single-stranded RNA. These terms include, as equivalents, analogs of either RNA or DNA made from nucleotide analogs and modified polynucleotides such as, though not limited to, methylated, protected and/or capped nucleotides or polynucleotides. The terms encompass poly- or oligo-ribonucleotides (RNA) and poly- or oligo-deoxyribonucleotides (DNA); RNA or DNA derived from N-glycosides or C-glycosides of nucleobases and/or modified nucleobases; nucleic acids derived from sugars and/or modified sugars; and nucleic acids derived from phosphate bridges and/or modified phosphorus-atom bridges (also referred to herein as “internucleotide linkages”). The term encompasses nucleic acids containing any combinations of nucleobases, modified nucleobases, sugars, modified sugars, phosphate bridges or modified phosphorus atom bridges. Examples include, and are not limited to, nucleic acids containing ribose moieties, the nucleic acids containing deoxy-ribose moieties, nucleic acids containing both ribose and deoxyribose moieties, nucleic acids containing ribose and modified ribose moieties. In some embodiments, the prefix poly-refers to a nucleic acid containing 2 to about 10,000, 2 to about 50,000, or 2 to about 100,000 nucleotide monomer units. In some embodiments, the prefix oligo-refers to a nucleic acid containing 2 to about 200 nucleotide monomer units.

Vector: As used herein, the term “vector” refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “expression vectors.”

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al., Molecular Cloning: A Laboratory Manual (2d ed., Cold Spring Harbor Laboratory Press, Cold Spring Harbor, N.Y. (1989)), which is incorporated herein by reference for any purpose.

### BRIEF DESCRIPTION OF THE DRAWING

FIG. 1 shows a chart that discloses example modification patterns 1-5 of the sense and antisense strands of the duplex of an inhibitory RNA (e.g., an siRNA). In FIG. 1, “2OM” represents a 2′-O-methyl modification, “2F” represents a 2′-Fluoro modification, and “PS” represents a phosphorothioate bond with the adjacent 3′ nucleotide.

FIG. 2 shows the structure of pegcetacoplan (“APL-2”), assuming n of about 800 to about 1100 and a PEG of about 40 kD.

### DETAILED DESCRIPTION OF CERTAIN EMBODIMENTS

### I. Complement System

To facilitate understanding of the disclosure, and without intending to limit the invention in any way, this section provides an overview of complement and its pathways of activation. Further details are found, e.g., in Kuby Immunology, 6th ed., 2006; Paul, W. E., Fundamental Immunology, Lippincott Williams & Wilkins; 6th ed., 2008; and Walport M J., Complement. First of two parts. N Engl J Med., 344 (14): 1058-66, 2001.

Complement is an arm of the innate immune system that plays an important role in defending the body against infectious agents. The complement system comprises more than 30 serum and cellular proteins that are involved in three major pathways, known as the classical, alternative, and lectin pathways. The classical pathway is usually triggered by binding of a complex of antigen and IgM or IgG antibody to C1 (though certain other activators can also initiate the pathway). Activated C1 cleaves C4 and C2 to produce C4a and C4b, in addition to C2a and C2b. C4b and C2a combine to form C3 convertase, which cleaves C3 to form C3a and C3b. Binding of C3b to C3 convertase produces C5 convertase, which cleaves C5 into C5a and C5b. C3a, C4a, and C5a are anaphylotoxins and mediate multiple reactions in the acute inflammatory response. C3a and C5a are also chemotactic factors that attract immune system cells such as neutrophils. It will be understood that the names “C2a” and “C2b” used initially were subsequently reversed in the scientific literature.

The alternative pathway is initiated by and amplified at, e.g., microbial surfaces and various complex polysaccharides. In this pathway, hydrolysis of C3 to C3 (H₂O), which occurs spontaneously at a low level, leads to binding of factor B, which is cleaved by factor D, generating a fluid phase C3 convertase that activates complement by cleaving C3 into C3a and C3b. C3b binds to targets such as cell surfaces and forms a complex with factor B, which is later cleaved by factor D, resulting in a C3 convertase. Surface-bound C3 convertases cleave and activate additional C3 molecules, resulting in rapid C3b deposition in close proximity to the site of activation and leading to formation of additional C3 convertase, which in turn generates additional C3b. This process results in a cycle of C3 cleavage and C3 convertase formation that significantly amplifies the response. Cleavage of C3 and binding of another molecule of C3b to the C3 convertase gives rise to a C5 convertase. C3 and C5 convertases of this pathway are regulated by cellular molecules CR1, DAF, MCP, CD59, and fH. The mode of action of these proteins involves either decay accelerating activity (i.e., ability to dissociate convertases), ability to serve as cofactors in the degradation of C3b or C4b by factor I, or both. Normally the presence of complement regulatory proteins on cell surfaces prevents significant complement activation from occurring thereon.

The C5 convertases produced in both pathways cleave C5 to produce C5a and C5b. C5b then binds to C6, C7, and C8 to form C5b-8, which catalyzes polymerization of C9 to form the C5b-9 membrane attack complex (MAC). The MAC inserts itself into target cell membranes and causes cell lysis. Small amounts of MAC on the membrane of cells may have a variety of consequences other than cell death.

The lectin complement pathway is initiated by binding of mannose-binding lectin (MBL) and MBL-associated serine protease (MASP) to carbohydrates. The MB1-1 gene (known as LMAN-1 in humans) encodes a type I integral membrane protein localized in the intermediate region between the endoplasmic reticulum and the Golgi. The MBL-2 gene encodes the soluble mannose-binding protein found in serum. In the human lectin pathway, MASP-1 and MASP-2 are involved in the proteolysis of C4 and C2, leading to a C3 convertase described above.

Complement activity is regulated by various mammalian proteins referred to as complement control proteins (CCPs) or regulators of complement activation (RCA) proteins (U.S. Pat. No. 6,897,290). These proteins differ with respect to ligand specificity and mechanism(s) of complement inhibition. They may accelerate the normal decay of convertases and/or function as cofactors for factor I, to enzymatically cleave C3b and/or C4b into smaller fragments. CCPs are characterized by the presence of multiple (typically 4-56) homologous motifs known as short consensus repeats (SCR), complement control protein (CCP) modules, or SUSHI domains, about 50-70 amino acids in length that contain a conserved motif including four disulfide-bonded cysteines (two disulfide bonds), proline, tryptophan, and many hydrophobic residues. The CCP family includes complement receptor type 1 (CR1; C3b: C4b receptor), complement receptor type 2 (CR2), membrane cofactor protein (MCP; CD46), decay-accelerating factor (DAF), complement factor H (fH), and C4b-binding protein (C4 bp). CD59 is a membrane-bound complement regulatory protein unrelated structurally to the CCPs. Complement regulatory proteins normally serve to limit complement activation that might otherwise occur on cells and tissues of the mammalian, e.g., human host. Thus, “self” cells are normally protected from the deleterious effects that would otherwise ensue were complement activation to proceed on these cells. Deficiencies or defects in complement regulatory protein(s) are involved in the pathogenesis of a variety of complement-mediated disorders, e.g., as discussed herein.

### II. Inhibitory RNAs for Complement Factor B

The disclosure includes compositions and methods related to one or more nucleotide sequences that are, comprise, or encode an inhibitory RNA that binds to and inhibits expression of messenger RNA (mRNA) produced by a target gene (e.g., factor B). Inhibitory RNAs can be single stranded (e.g., an antisense oligonucleotide) or double stranded nucleic acid. In some embodiments, an inhibitory RNA comprises a double stranded RNA duplex such as microRNA (miRNA) or small interfering RNA (siRNA). In some embodiments, in inhibitory RNA is an siRNA or miRNA, or a vector comprising a nucleotide sequence encoding an siRNA or miRNA.

In some embodiments, an inhibitory RNA is capable of inhibiting expression of factor B of one or more non-human species, e.g., a non-human primate factor B, e.g., Macaca fascicularis factor B, or e.g., Chlorocebus sabaeus in addition to human factor B. The Macaca fascicularis factor B gene has been assigned NCBI Gene ID: 102133787 and the predicted amino acid and nucleotide sequence of Macaca fascicularis factor B are listed under NCBI RefSeq accession numbers XP_005553497.1 (derived from genomic sequence NW_005092966.1) and XM_005553440.2, respectively. In some embodiments, an inhibitory RNA comprises an antisense strand that is complementary to a target portion that is identical in the human and Macaca fascicularis factor B transcripts. In some embodiments, an inhibitory RNA comprises an antisense strand that is complementary to a target portion of a human factor B transcript that differs by 1, 2, or 3 nucleotides from a sequence in a Macaca fascicularis fact or B transcript. It will be appreciated that an inhibitory RNA that inhibits expression of human factor B may also inhibit expression of non-primate factor B, e.g., rat or mouse factor B, particularly if conserved regions of factor B transcript are targeted.

The amino acid and nucleotide sequences of human factor B are known in the art and can be found in publicly available databases, for example, the National Center for Biotechnology Information (NCBI) Reference Sequence (RefSeq) database, where they are listed under RefSeq accession numbers NP_001701 (accession. version number NP_001701.2) and NM_001710 (accession. version number NM_001710.6), respectively (where “amino acid sequence” refers to the sequence of the factor B polypeptide and “nucleotide sequence” in this context refers to the factor B mRNA sequence as represented in genomic DNA, it being understood that the actual mRNA nucleotide sequence contains U rather than T). One of ordinary skill in the art will appreciate that the afore-mentioned sequences are for the complement factor B preproprotein, which includes a signal sequence that is cleaved off and is therefore not present in the mature protein. The human factor B gene has been assigned NCBI Gene ID: 629, and the genomic factor B sequence has RefSeq accession number NG_008191 (accession. version number NG_008191.1). The nucleotide sequence of human factor B mRNA is presented below (from RefSeq accession number NM_001710.6).

(SEQ ID NO: 75)
GGGAAGGGAAUGUGACCAGGUCUAGGUCUGGAGUUUCAGCUUGGACACUGAGCCAAGCAGACAA
GCAAAGCAAGCCAGGACACACCAUCCUGCCCCAGGCCCAGCUUCUCUCCUGCCUUCCAACGCCA
UGGGGAGCAAUCUCAGCCCCCAACUCUGCCUGAUGCCCUUUAUCUUGGGCCUCUUGUCUGGAGG
UGUGACCACCACUCCAUGGUCUUUGGCCCGGCCCCAGGGAUCCUGCUCUCUGGAGGGGGUAGAG
AUCAAAGGCGGCUCCUUCCGACUUCUCCAAGAGGGCCAGGCACUGGAGUACGUGUGUCCUUCUG
GCUUCUACCCGUACCCUGUGCAGACACGUACCUGCAGAUCUACGGGGUCCUGGAGCACCCUGAA
GACUCAAGACCAAAAGACUGUCAGGAAGGCAGAGUGCAGAGCAAUCCACUGUCCAAGACCACAC
GACUUCGAGAACGGGGAAUACUGGCCCCGGUCUCCCUACUACAAUGUGAGUGAUGAGAUCUCUU
UCCACUGCUAUGACGGUUACACUCUCCGGGGCUCUGCCAAUCGCACCUGCCAAGUGAAUGGCCG
AUGGAGUGGGCAGACAGCGAUCUGUGACAACGGAGCGGGGUACUGCUCCAACCCGGGCAUCCCC
AUUGGCACAAGGAAGGUGGGCAGCCAGUACCGCCUUGAAGACAGCGUCACCUACCACUGCAGCC
GGGGGCUUACCCUGCGUGGCUCCCAGCGGCGAACGUGUCAGGAAGGUGGCUCUUGGAGCGGGAC
GGAGCCUUCCUGCCAAGACUCCUUCAUGUACGACACCCCUCAAGAGGUGGCCGAAGCUUUCCUG
UCUUCCCUGACAGAGACCAUAGAAGGAGUCGAUGCUGAGGAUGGGCACGGCCCAGGGGAACAAC
AGAAGCGGAAGAUCGUCCUGGACCCUUCAGGCUCCAUGAACAUCUACCUGGUGCUAGAUGGAUC
AGACAGCAUUGGGGCCAGCAACUUCACAGGAGCCAAAAAGUGUCUAGUCAACUUAAUUGAGAAG
GUGGCAAGUUAUGGUGUGAAGCCAAGAUAUGGUCUAGUGACAUAUGCCACAUACCCCAAAAUUU
GGGUCAAAGUGUCUGAAGCAGACAGCAGUAAUGCAGACUGGGUCACGAAGCAGCUCAAUGAAAU
CAAUUAUGAAGACCACAAGUUGAAGUCAGGGACUAACACCAAGAAGGCCCUCCAGGCAGUGUAC
AGCAUGAUGAGCUGGCCAGAUGACGUCCCUCCUGAAGGCUGGAACCGCACCCGCCAUGUCAUCA
UCCUCAUGACUGAUGGAUUGCACAACAUGGGGGGGGACCCAAUUACUGUCAUUGAUGAGAUCCG
GGACUUGCUAUACAUUGGCAAGGAUCGCAAAAACCCAAGGGAGGAUUAUCUGGAUGUCUAUGUG
UUUGGGGUCGGGCCUUUGGUGAACCAAGUGAACAUCAAUGCUUUGGCUUCCAAGAAAGACAAUG
AGCAACAUGUGUUCAAAGUCAAGGAUAUGGAAAACCUGGAAGAUGUUUUCUACCAAAUGAUCGA
UGAAAGCCAGUCUCUGAGUCUCUGUGGCAUGGUUUGGGAACACAGGAAGGGUACCGAUUACCAC
AAGCAACCAUGGCAGGCCAAGAUCUCAGUCAUUCGCCCUUCAAAGGGACACGAGAGCUGUAUGG
GGGCUGUGGUGUCUGAGUACUUUGUGCUGACAGCAGCACAUUGUUUCACUGUGGAUGACAAGGA
ACACUCAAUCAAGGUCAGCGUAGGAGGGGAGAAGCGGGACCUGGAGAUAGAAGUAGUCCUAUUU
CACCCCAACUACAACAUUAAUGGGAAAAAAGAAGCAGGAAUUCCUGAAUUUUAUGACUAUGACG
UUGCCCUGAUCAAGCUCAAGAAUAAGCUGAAAUAUGGCCAGACUAUCAGGCCCAUUUGUCUCCC
CUGCACCGAGGGAACAACUCGAGCUUUGAGGCUUCCUCCAACUACCACUUGCCAGCAACAAAAG
GAAGAGCUGCUCCCUGCACAGGAUAUCAAAGCUCUGUUUGUGUCUGAGGAGGAGAAAAAGCUGA
CUCGGAAGGAGGUCUACAUCAAGAAUGGGGAUAAGAAAGGCAGCUGUGAGAGAGAUGCUCAAUA
UGCCCCAGGCUAUGACAAAGUCAAGGACAUCUCAGAGGUGGUCACCCCUCGGUUCCUUUGUACU
GGAGGAGUGAGUCCCUAUGCUGACCCCAAUACUUGCAGAGGUGAUUCUGGCGGCCCCUUGAUAG
UUCACAAGAGAAGUCGUUUCAUUCAAGUUGGUGUAAUCAGCUGGGGAGUAGUGGAUGUCUGCAA
AAACCAGAAGCGGCAAAAGCAGGUACCUGCUCACGCCCGAGACUUUCACAUCAACCUCUUUCAA
GUGCUGCCCUGGCUGAAGGAGAAACUCCAAGAUGAGGAUUUGGGUUUUCUAUAAGGGGUUUCCU
GCUGGACAGGGGCGUGGGAUUGAAUUAAAACAGCUGCGACAACA

In some embodiments, an inhibitory RNA comprises a nucleic acid strand that is complementary to a target portion of a factor B transcript, e.g., factor B mRNA (e.g., complementary to a nucleotide sequence that is at least 90%, 91%, 92%, 93%, 94%, 95%, 96%, 97%, 98%, 99%, or 100% identical to a target portion of SEQ ID NO: 75). The target portion may be 15-30 nucleotides long, e.g., 15, 16, 17, 18, 19, 20, 21, 22, 23, 24, 25, 26, 27, 28, 29, or 30 nucleotides long, although shorter and longer target portions are also contemplated. In some embodiments, the target portion comprises a sequence that is at least 90%, 91%, 92%, 93%, 94%, 95%, 96%, 97%, 98%, 99%, or 100% identical to any one of the sequences listed below in Table 1.

TABLE 1

| SEQ ID | Target Sequence |
|---|---|
| NO: | (5′ TO 3′) |
| 76 | CCUACUACAAUGUGAGUG |
| 77 | CUUCUGGCUUCUACCCGU |
| 78 | UUCUGGCUUCUACCCGUA |
| 79 | CUGCUAUGACGGUUACAC |
| 80 | GGGGAGUAGUGGAUGUCU |
| 81 | UGUGGUGUCUGAGUACUU |
| 82 | UACGUGUGUCCUUCUGGC |
| 83 | AAGUGUCUAGUCAACUUA |
| 84 | CUAUGACGUUGCCCUGAU |
| 85 | CUCGGUUCCUUUGUACUG |
| 86 | ACCAUAGAAGGAGUCGAU |
| 87 | GGAUUAUCUGGAUGUCUA |
| 88 | ACGUUGCCCUGAUCAAGC |
| 89 | GGAGCCAAAAAGUGUCUA |
| 90 | CCGAUUACCACAAGCAAC |
| 91 | AAUGCAGACUGGGUCACG |
| 92 | CUCACGCCCGAGACUUUC |
| 93 | CAGCGAUCUGUGACAACG |
| 94 | CUACCACUUGCCAGCAAC |
| 95 | UUUAUGACUAUGACGUUG |
| 96 | CCUUGAUAGUUCACAAGA |
| 97 | CUCAAGAGGUGGCCGAAG |
| 98 | UUUUAUGACUAUGACGUU |
| 99 | UUCCGACUUCUCCAAGAG |

Administration of an inhibitory RNA can reduce the level of factor B transcript or factor B protein in the subject or in a biological sample (e.g., a blood, serum or plasma sample, or a sample comprising hepatocytes) compared to a level before the administration of the composition. In some embodiments, the level of factor B transcript or factor B protein is reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90%, relative to a level before the administration. Level of factor B protein can be measured, for example, in a blood (serum or plasma) sample.

### III. MicroRNAs

The disclosure also includes compositions and methods related to one or more oligonucleotides that are, comprise, or encode, microRNAs. MicroRNAs (miRNAs) are a highly conserved class of small RNA molecules that are transcribed from DNA in the genomes of plants and animals, but are not translated into protein. Naturally occurring miRNAs are first transcribed as long hairpin-containing primary transcripts (pri-miRNAs). The primary transcript is cleaved by Drosha ribonuclease III enzyme to produce an approximately 70 nt stem-loop precursor miRNA (pre-miRNA), which includes an “antisense strand” or “guide strand” (that includes a region that is substantially complementary to a target sequence) and a “sense strand” or “passenger strand” (that includes a region that is substantially complementary to a region of the antisense strand). The pre-miRNA is then actively exported to the cytoplasm where it is cleaved by Dicer ribonuclease to form the mature miRNA. Processed microRNAs are incorporated into the RNA-induced silencing complex (RISC) to form mature gene-silencing complexes, which induce target mRNA degradation and/or translation repression. The number of miRNA sequences identified to date is large and growing, illustrative examples of which can be found, for example, in: “miRBase: microRIVA sequences, targets and gene nomenclature” Griffiths-Jones S, Grocock R J, van Dongen S, Bateman A, Enright A J. NAR, 2006, 34, Database Issue, D140-D144; “The microRNA Registry” Griffiths-Jones S. NAR, 2004, 32, Database Issue, D109-D111.

In some embodiments, miRNAs can be synthesized and locally or systemically administered to a subject, e.g., for therapeutic purposes. miRNAs can be designed and/or synthesized as mature molecules or precursors (e.g., pri- or pre-miRNAs). In some embodiments, a pre-miRNA includes a guide strand and a passenger strand that are the same length (e.g., about 15, 16, 17, 18, 19, 20, 21, 22, 23, 24, or 25 nucleotides). In some embodiments, a pre-miRNA includes a guide strand and a passenger strand that are different lengths (e.g., one strand is about 19 nucleotides, and the other is about 21 nucleotides). In some embodiments, an miRNA can target the coding region, the 5′ untranslated region, and/or 3′ untranslated region, of endogenous mRNA. In some embodiments, an miRNA comprises a guide strand comprising a nucleotide sequence having sufficient sequence complementary with an endogenous mRNA of a subject to hybridize with and inhibit expression of the endogenous mRNA.

In some embodiments, the miRNA comprises a nucleic acid strand that comprises a region that is perfectly complementary to at least 6, 7, 8, 9, 10, 11, 12, 13 14, 15, 16, 17, 18, 19, 20, 21, 22, 23, 24, 25, 26, 27, 28, 29, or 30 consecutive nucleotides of SEQ ID NO: 75 (e.g., any one of SEQ ID NOs: 76-99). In some embodiments, an miRNA comprises a mature guide strand having a nucleotide sequence that is perfectly complementary to a target portion comprising a sequence that is at least 90%, 91%, 92%, 93%, 94%, 95%, 96%, 97%, 98%, 99%, or 100% identical to any one of SEQ ID NOs: 76-99.

### IV. SiRNAs

In some embodiments, an inhibitory RNA is a double stranded RNA (dsRNA), and inhibits factor B expression by RNA interference (RNAi). RNAi is a process of sequence-specific post-transcriptional gene silencing by which, e.g., double stranded RNA (dsRNA) homologous to a target locus can specifically inactivate gene function (Hammond et al., Nature Genet. 2001; 2:110-119; Sharp, Genes Dev. 1999; 13:139-141). This dsRNA-induced gene silencing can be mediated by short double-stranded small interfering RNAs (siRNAs) generated from longer dsRNAs by ribonuclease III cleavage (Bernstein et al., Nature 2001; 409:363-366 and Elbashir et al., Genes Dev. 2001; 15:188-200). RNAi-mediated gene silencing is thought to occur via sequence-specific RNA degradation, where sequence specificity is determined by the interaction of an siRNA with its complementary sequence within a target RNA (see, e.g., Tuschl, Chem. Biochem. 2001; 2:239-245). RNAi can involve the use of, e.g., siRNAs (Elbashir, et al., Nature 2001; 411:494-498) or short hairpin RNAs (shRNAs) bearing a fold back stem-loop structure (Paddison et al., Genes Dev. 2002; 16:948-958; Sui et al., Proc. Natl. Acad. Sci. USA 2002; 99:5515-5520; Brummelkamp et al., Science 2002; 296:550-553; Paul et al., Nature Biotechnol. 2002; 20:505-508).

The disclosure includes siRNA molecules targeting factor B transcript, e.g., factor B mRNA (SEQ ID NO: 75). In some embodiments, an siRNA molecule comprises a sequence that is complementary to a target region comprising any one of SEQ ID NOs: 76-99. In some embodiments, an siRNA molecule comprises (i) a nucleotide sequence that is at least 90%, 91%, 92%, 93%, 94%, 95%, 96%, 97%, 98%, 99%, or 100% identical to any one of SEQ ID NOs: 76-99 (or a portion thereof) and/or (ii) a nucleotide sequence that is complementary to a nucleotide sequence that is at least 90%, 91%, 92%, 93%, 94%, 95%, 96%, 97%, 98%, 99%, or 100% identical to any one of SEQ ID NOs: 76-99 (or a portion thereof).

In some embodiments, siRNAs of the disclosure are double stranded nucleic acid duplexes (of, e.g., 15, 16, 17, 18, 19, 20, 21, 22, 23, 24, 25, 26, or 27 base pairs) comprising annealed complementary single stranded nucleic acid molecules. In some embodiments, the siRNAs are short dsRNAs comprising annealed complementary single strand RNAs. In some embodiments, the siRNAs comprise an annealed RNA: DNA duplex, wherein the sense strand of the duplex is a DNA molecule and the antisense strand of the duplex is a RNA molecule. In some embodiments, an siRNA comprises a sense strand having a nucleotide sequence that is at least 90%, 91%, 92%, 93%, 94%, 95%, 96%, 97%, 98%, 99%, or 100% identical to any one of SEQ ID NOs: 76-99 (or a portion thereof).

In some embodiments, an siRNA comprises an antisense strand having a nucleotide sequence that is at least 90%, 91%, 92%, 93%, 94%, 95%, 96%, 97%, 98%, 99%, or 100% identical to any one of SEQ ID NOs: 100-123 in the following Table 2:

TABLE 2

| SEQ ID | Antisense Sequence |
|---|---|
| NO: | (5′ TO 3′) |
| 100 | CACUCACAUUGUAGUAGG |
| 101 | ACGGGUAGAAGCCAGAAG |
| 102 | UACGGGUAGAAGCCAGAA |
| 103 | GUGUAACCGUCAUAGCAG |
| 104 | AGACAUCCACUACUCCCC |
| 105 | AAGUACUCAGACACCACA |
| 106 | GCCAGAAGGACACACGUA |
| 107 | UAAGUUGACUAGACACUU |
| 108 | AUCAGGGCAACGUCAUAG |
| 109 | CAGUACAAAGGAACCGAG |
| 110 | AUCGACUCCUUCUAUGGU |
| 111 | UAGACAUCCAGAUAAUCC |
| 112 | GCUUGAUCAGGGCAACGU |
| 113 | UAGACACUUUUUGGCUCC |
| 114 | GUUGCUUGUGGUAAUCGG |
| 115 | CGUGACCCAGUCUGCAUU |
| 116 | GAAAGUCUCGGGCGUGAG |
| 117 | CGUUGUCACAGAUCGCUG |
| 118 | GUUGCUGGCAAGUGGUAG |
| 119 | CAACGUCAUAGUCAUAAA |
| 120 | UCUUGUGAACUAUCAAGG |
| 121 | CUUCGGCCACCUCUUGAG |
| 122 | AACGUCAUAGUCAUAAAA |
| 123 | CUCUUGGAGAAGUCGGAA |

In some embodiments, an siRNA comprises mismatch(es) with the target, within the duplex, or combinations thereof. The mismatch may occur in an overhang region and/or the duplex portion. The base pair may be ranked on the basis of their propensity to promote dissociation or melting (e.g., on the free energy of association or dissociation of a particular pairing, the simplest approach is to examine the pairs on an individual pair basis, though next neighbor or similar analysis can also be used). In terms of promoting dissociation: A: U is preferred over G: C; G: U is preferred over G: C; and I: C is preferred over G: C (I-inosine).

In some embodiments, an siRNA comprises at least one of the first 1, 2, 3, 4, or 5 base pairs within the duplex portions from the 5′-end of the antisense strand independently selected from the group of: A: U, G: U, I: C, and mismatched pairs. In some embodiments, the nucleotide at the 1 position within the duplex portion from the 5′-end in the antisense strand is selected from the group consisting of A, dA, dU, U, and dT. Additionally or alternatively, at least one of the first 1, 2 or 3 base pairs within the duplex portion from the 5′-end of the antisense strand is an AU base pair. For example, the first base pair within the duplex portion from the 5′-end of the antisense strand is an AU base pair.

In some embodiments, a sense strand can include one or more (e.g., 2, 3, 4, or 5) nucleotides on the 3′ and/or 5′ end that is not identical to the target sequence, and/or an antisense strand can include one or more (e.g., 2, 3, 4, or 5) nucleotides on the 3′ and/or 5′ end that is not complementary to the target sequence. For example, in some embodiments, a duplexed siRNA comprises a sense strand comprising a sequence that contains an additional adenine (A) nucleotide at its 3′ end and an antisense strand that is complementary to the sense strand. In some embodiments, a duplexed siRNA comprises a sense strand comprising a sequence listed in the following Table 3. In some embodiments, a duplexed siRNA comprises an antisense strand that is complementary to a sense strand sequence listing in Table 3. The sequences in Table 3 contain an adenine (A) nucleotide at the 3′ end, which, in some of the sequences, is identical to the target sequence (e.g., is identical to the next contiguous nucleotide of the target sequence). In some of the sequences in Table 3, the adenine (A) nucleotide at the 3′ end is not identical to the target sequence (e.g., is not identical to the next contiguous nucleotide of the target sequence).

TABLE 3

| SEQ ID | Sense Sequence |
|---|---|
| NO: | (5′ to 3′) |
| 124 | CCUACUACAAUGUGAGUGA |
| 125 | CUUCUGGCUUCUACCCGUA |
| 126 | UUCUGGCUUCUACCCGUAA |
| 127 | CUGCUAUGACGGUUACACA |
| 128 | GGGGAGUAGUGGAUGUCUA |
| 129 | UGUGGUGUCUGAGUACUUA |
| 130 | UACGUGUGUCCUUCUGGCA |
| 131 | AAGUGUCUAGUCAACUUAA |
| 132 | CUAUGACGUUGCCCUGAUA |
| 133 | CUCGGUUCCUUUGUACUGA |
| 134 | ACCAUAGAAGGAGUCGAUA |
| 135 | GGAUUAUCUGGAUGUCUAA |
| 136 | ACGUUGCCCUGAUCAAGCA |
| 137 | GGAGCCAAAAAGUGUCUAA |
| 138 | CCGAUUACCACAAGCAACA |
| 139 | AAUGCAGACUGGGUCACGA |
| 140 | CUCACGCCCGAGACUUUCA |
| 141 | CAGCGAUCUGUGACAACGA |
| 142 | CUACCACUUGCCAGCAACA |
| 143 | UUUAUGACUAUGACGUUGA |
| 144 | CCUUGAUAGUUCACAAGAA |
| 145 | CUCAAGAGGUGGCCGAAGA |
| 146 | UUUUAUGACUAUGACGUUA |
| 147 | UUCCGACUUCUCCAAGAGA |

In some embodiments, duplexed siRNAs comprise blunt ends on both ends. In some embodiments, duplexed siRNAs comprise at least one overhang region. In some embodiments, a duplexed siRNA comprises a 1, 2, 3, 4, 5, or 6 nucleotide 3′ overhang on the sense and/or antisense strand of the duplex. In some embodiments, a duplexed siRNA comprises a 1, 2, 3, 4, 5, or 6 nucleotide 5′ overhang on the sense and/or antisense strand of the duplex.

In some embodiments, an antisense strand comprises an overhang comprising one or more nucleotides that are complementary to the factor B mRNA transcript (SEQ ID NO: 75). In some embodiments, an antisense strand comprises an overhang comprising 1, 2, 3, 4, 5, or 6 nucleotides that are complementary to the factor B mRNA transcript (SEQ ID NO: 75). In some embodiments, an antisense strand comprises a 3′ overhang that comprises 2 nucleotides that are complementary to the factor B mRNA transcript (SEQ ID NO: 75). In some embodiments, an antisense strand comprises a 5′ uracil (U) nucleotide and/or a 3′ overhang that comprises 2 nucleotides that are complementary to the factor B mRNA transcript (SEQ ID NO: 75). For example, in some embodiments, a duplexed siRNA comprises an antisense strand comprising the sequence of any one of SEQ ID NOs: 148-171.

TABLE 4

| SEQ ID | Antisense Sequence |
|---|---|
| NO: | (5′ TO 3′) |
| 148 | UCACUCACAUUGUAGUAGGGA |
| 149 | UACGGGUAGAAGCCAGAAGGA |
| 150 | UUACGGGUAGAAGCCAGAAGG |
| 151 | UGUGUAACCGUCAUAGCAGUG |
| 152 | UAGACAUCCACUACUCCCCAG |
| 153 | UAAGUACUCAGACACCACAGC |
| 154 | UGCCAGAAGGACACACGUACU |
| 155 | UUAAGUUGACUAGACACUUUU |
| 156 | UAUCAGGGCAACGUCAUAGUC |
| 157 | UCAGUACAAAGGAACCGAGGG |
| 158 | UAUCGACUCCUUCUAUGGUCU |
| 159 | UUAGACAUCCAGAUAAUCCUC |
| 160 | UGCUUGAUCAGGGCAACGUCA |
| 161 | UUAGACACUUUUUGGCUCCUG |
| 162 | UGUUGCUUGUGGUAAUCGGUA |
| 163 | UCGUGACCCAGUCUGCAUUAC |
| 164 | UGAAAGUCUCGGGCGUGAGCA |
| 165 | UCGUUGUCACAGAUCGCUGUC |
| 166 | UGUUGCUGGCAAGUGGUAGUU |
| 167 | UCAACGUCAUAGUCAUAAAAU |
| 168 | UUCUUGUGAACUAUCAAGGGG |
| 169 | UCUUCGGCCACCUCUUGAGGG |
| 170 | UAACGUCAUAGUCAUAAAAUU |
| 171 | UCUCUUGGAGAAGUCGGAAGG |

In some embodiments, a duplexed siRNA comprises an antisense strand comprising the sequence of any one of SEQ ID NOs: 148-171, but lacking the “U” at the 5′ end.

In some embodiments, an antisense strand comprises an overhang comprising one or more nucleotides that are not complementary to the factor B mRNA transcript (SEQ ID NO: 75). In some embodiments, an antisense strand comprises an overhang comprising 1, 2, 3, 4, 5, or 6 nucleotides that are not complementary to the factor B mRNA transcript (SEQ ID NO: 75). In one example, an overhang comprises a 3′ overhang on the antisense and/or sense strand including 1, 2, or 3 uracil nucleotides. In one example, an overhang comprises a 3′ overhang on the antisense and/or sense strand including 1, 2, or 3 adenine nucleotides.

In some embodiments, an antisense strand comprises an overhang that comprises 2 uracil (U) nucleotides. For example, in some embodiments, a duplexed siRNA comprises an antisense strand comprising a sequence listed in the following Table 5:

TABLE 5

| SEQ ID | Antisense Sequence |
|---|---|
| NO: | (5′ TO 3′) |
| 172 | CACUCACAUUGUAGUAGGUU |
| 173 | ACGGGUAGAAGCCAGAAGUU |
| 174 | UACGGGUAGAAGCCAGAAUU |
| 175 | GUGUAACCGUCAUAGCAGUU |
| 176 | AGACAUCCACUACUCCCCUU |
| 177 | AAGUACUCAGACACCACAUU |
| 178 | GCCAGAAGGACACACGUAUU |
| 179 | UAAGUUGACUAGACACUUUU |
| 180 | AUCAGGGCAACGUCAUAGUU |
| 181 | CAGUACAAAGGAACCGAGUU |
| 182 | AUCGACUCCUUCUAUGGUUU |
| 183 | UAGACAUCCAGAUAAUCCUU |
| 184 | GCUUGAUCAGGGCAACGUUU |
| 185 | UAGACACUUUUUGGCUCCUU |
| 186 | GUUGCUUGUGGUAAUCGGUU |
| 187 | CGUGACCCAGUCUGCAUUUU |
| 188 | GAAAGUCUCGGGCGUGAGUU |
| 189 | CGUUGUCACAGAUCGCUGUU |
| 190 | GUUGCUGGCAAGUGGUAGUU |
| 191 | CAACGUCAUAGUCAUAAAUU |
| 192 | UCUUGUGAACUAUCAAGGUU |
| 193 | CUUCGGCCACCUCUUGAGUU |
| 194 | AACGUCAUAGUCAUAAAAUU |
| 195 | CUCUUGGAGAAGUCGGAAUU |

In some embodiments, an antisense strand comprises a 5′ uracil (U) nucleotide and a 3′ overhang that comprises 2 uracil (U) nucleotides. For example, in some embodiments, a duplexed siRNA comprises an antisense strand comprises a sequence listed in the following Table 6:

TABLE 6

| SEQ ID | Antisense Sequence |
|---|---|
| NO: | (5′ TO 3′) |
| 196 | UCACUCACAUUGUAGUAGGUU |
| 197 | UACGGGUAGAAGCCAGAAGUU |
| 198 | UUACGGGUAGAAGCCAGAAUU |
| 199 | UGUGUAACCGUCAUAGCAGUU |
| 200 | UAGACAUCCACUACUCCCCUU |
| 201 | UAAGUACUCAGACACCACAUU |
| 202 | UGCCAGAAGGACACACGUAUU |
| 203 | UUAAGUUGACUAGACACUUUU |
| 204 | UAUCAGGGCAACGUCAUAGUU |
| 205 | UCAGUACAAAGGAACCGAGUU |
| 206 | UAUCGACUCCUUCUAUGGUUU |
| 207 | UUAGACAUCCAGAUAAUCCUU |
| 208 | UGCUUGAUCAGGGCAACGUUU |
| 209 | UUAGACACUUUUUGGCUCCUU |
| 210 | UGUUGCUUGUGGUAAUCGGUU |
| 211 | UCGUGACCCAGUCUGCAUUUU |
| 212 | UGAAAGUCUCGGGCGUGAGUU |
| 213 | UCGUUGUCACAGAUCGCUGUU |
| 214 | UGUUGCUGGCAAGUGGUAGUU |
| 215 | UCAACGUCAUAGUCAUAAAUU |
| 216 | UUCUUGUGAACUAUCAAGGUU |
| 217 | UCUUCGGCCACCUCUUGAGUU |
| 218 | UAACGUCAUAGUCAUAAAAUU |
| 219 | UCUCUUGGAGAAGUCGGAAUU |

In some embodiments, siRNAs comprise 5′-phosphate and/or 3′-hydroxyl (e.g., on one or both ends of a sense strand and/or on one or both ends of an antisense strand) groups and/or may comprise one or more additional modifications described herein.

### V. Modifications

In some embodiments, an inhibitory RNA (e.g., an siRNA or miRNA) of the disclosure includes one or more natural nucleobase and/or one or more modified nucleobases derived from a natural nucleobase. Examples include, but are not limited to, uracil, thymine, adenine, cytosine, and guanine having their respective amino groups protected by acyl protecting groups, 2-fluorouracil, 2-fluorocytosine, 5-bromouracil, 5-iodouracil, 2,6-diaminopurine, azacytosine, pyrimidine analogs such as pseudoisocytosine and pseudouracil and other modified nucleobases such as 8-substituted purines, xanthine, or hypoxanthine (the latter two being the natural degradation products). Exemplary modified nucleobases are disclosed in Chiu and Rana, R N A, 2003, 9, 1034-1048, Limbach et al. Nucleic Acids Research, 1994, 22, 2183-2196 and Revankar and Rao, Comprehensive Natural Products Chemistry, vol. 7, 313.

Modified nucleobases also include expanded-size nucleobases in which one or more aryl rings, such as phenyl rings, have been added. Nucleic base replacements described in the Glen Research catalog (www.glenresearch.com); Krueger A T et al, Acc. Chem. Res., 2007, 40, 141-150; Kool, ET, Acc. Chem. Res., 2002, 35, 936-943; Benner S. A., et al., Nat. Rev. Genet., 2005, 6, 553-543; Romesberg, F. E., et al., Curr. Opin. Chem. Biol., 2003, 7, 723-733; Hirao, I., Curr. Opin. Chem. Biol., 2006, 10, 622-627, are contemplated as useful for siRNA molecules described herein. Modified nucleobases also encompass structures that are not considered nucleobases but are other moieties such as, but not limited to, corrin- or porphyrin-derived rings. Porphyrin-derived base replacements have been described in Morales-Rojas, H and Kool, ET, Org. Lett., 2002, 4, 4377-4380.

In some embodiments, modified nucleobases are of any one of the following structures, optionally substituted:

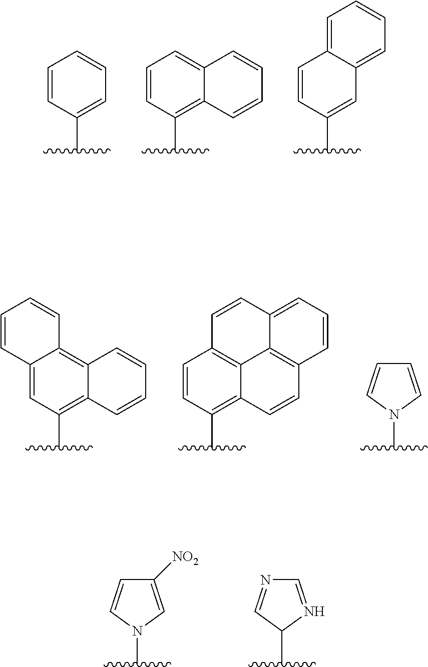

In some embodiments, a modified nucleobase is fluorescent. Exemplary such fluorescent modified nucleobases include phenanthrene, pyrene, stillbene, isoxanthine, isozanthopterin, terphenyl, terthiophene, benzoterthiophene, coumarin, lumazine, tethered stillbene, benzo-uracil, and naphtho-uracil, as shown below:

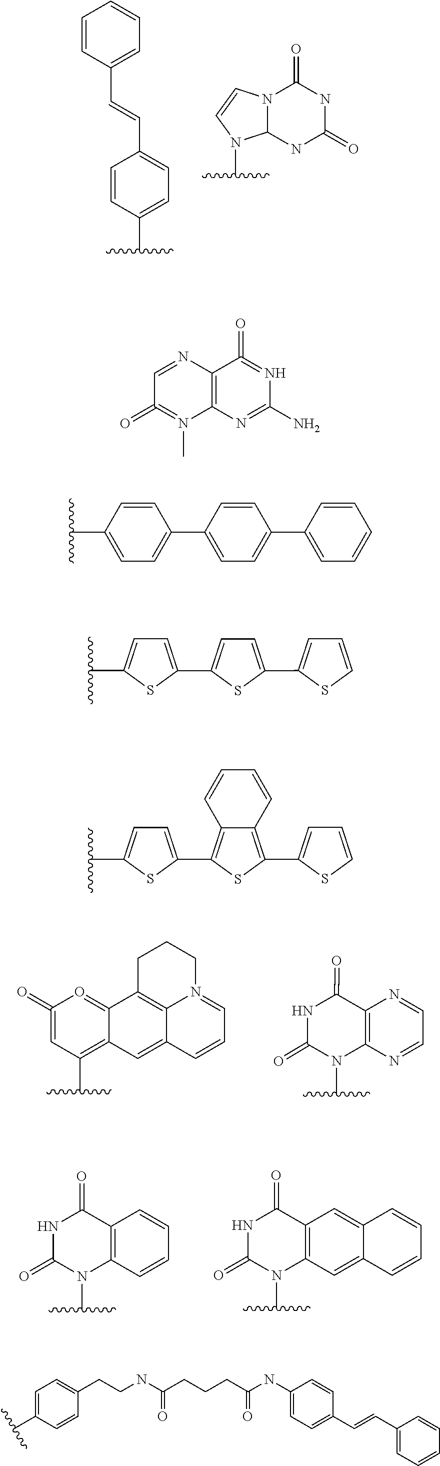

In some embodiments, a modified nucleobase is unsubstituted. In some embodiments, a modified nucleobase is substituted. In some embodiments, a modified nucleobase is substituted such that it contains, e.g., heteroatoms, alkyl groups, or linking moieties connected to fluorescent moieties, biotin or avidin moieties, or other protein or peptides. In some embodiments, a modified nucleobase is a “universal base” that is not a nucleobase in the most classical sense, but that functions similarly to a nucleobase. One representative example of such a universal base is 3-nitropyrrole.

In some embodiments, an siRNA described herein includes nucleosides that incorporate modified nucleobases and/or nucleobases covalently bound to modified sugars. Some examples of nucleosides that incorporate modified nucleobases include 4-acetylcytidine; 5-(carboxyhydroxylmethyl) uridine; 2′-O-methylcytidine; 5-carboxymethylaminomethyl-2-thiouridine; 5-carboxymethylaminomethyluridine; dihydrouridine; 2′-O-methylpseudouridine; beta,D-galactosylqueosine; 2′-O-methylguanosine; N⁶-isopentenyladenosine; 1-methyladenosine; 1-methylpseudouridine; 1-methylguanosine; 1-methylinosine; 2,2-dimethylguanosine; 2-methyladenosine; 2-methylguanosine; N⁷-methylguanosine; 3-methyl-cytidine; 5-methylcytidine; 5-hydroxymethylcytidine; 5-formylcytosine; 5-carboxylcytosine; N⁶-methyladenosine; 7-methylguanosine; 5-methylaminoethyluridine; 5-methoxyaminomethyl-2-thiouridine; beta, D-mannosylqueosine; 5-methoxycarbonylmethyluridine; 5-methoxyuridine; 2-methylthio-N⁶-isopentenyladenosine; N-((9-beta, D-ribofuranosyl-2-methylthiopurine-6-yl) carbamoyl) threonine; N-((9-beta,D-ribofuranosylpurine-6-yl)-N-methylcarbamoyl) threonine; uridine-5-oxyacetic acid methylester; uridine-5-oxyacetic acid (v); pseudouridine; queosine; 2-thiocytidine; 5-methyl-2-thiouridine; 2-thiouridine; 4-thiouridine; 5-methyluridine; 2′-O-methyl-5-methyluridine; and 2′-O-methyluridine.

In some embodiments, nucleosides include 6′-modified bicyclic nucleoside analogs that have either (R) or(S)-chirality at the 6′-position and include the analogs described in U.S. Pat. No. 7,399,845. In other embodiments, nucleosides include 5′-modified bicyclic nucleoside analogs that have either (R) or(S)-chirality at the 5′-position and include the analogs described in U.S. Publ. No. 20070287831. In some embodiments, a nucleobase or modified nucleobase is 5-bromouracil, 5-iodouracil, or 2,6-diaminopurine. In some embodiments, a nucleobase or modified nucleobase is modified by substitution with a fluorescent moiety.

Methods of preparing modified nucleobases are described in, e.g., U.S. Pat. Nos. 3,687,808; 4,845,205; 5,130,30; 5,134,066; 5,175,273; 5,367,066:5,432,272:5,457,187:5,457,191; 5,459,255; 5,484,908; 5,502,177; 5,525,711; 5,552,540; 5,587,469; 5,594,121, 5,596,091; 5,614,617; 5,681,941; 5,750,692; 6,015,886; 6,147,200; 6,166,197; 6,222,025; 6,235,887; 6,380,368; 6,528,640; 6,639,062; 6,617,438; 7,045,610; 7,427,672; and 7,495,088.

In some embodiments, an siRNA described herein includes one or more modified nucleotides wherein a phosphate group or linkage phosphorus in the nucleotides are linked to various positions of a sugar or modified sugar. As non-limiting examples, the phosphate group or linkage phosphorus can be linked to the 2′, 3′, 4′ or 5′ hydroxyl moiety of a sugar or modified sugar. Nucleotides that incorporate modified nucleobases as described herein are also contemplated in this context.

Other modified sugars can also be incorporated within an siRNA molecule. In some embodiments, a modified sugar contains one or more substituents at the 2′ position including one of the following: —F; —CF₃, —CN, —N₃, —NO, —NO₂, —OR′, —SR′, or —N(R′)₂, wherein each R′ is independently as defined above and described herein; —O—(C₁-C₁₀ alkyl), —S—(C₁-C₁₀ alkyl), —NH—(C₁-C₁₀ alkyl), or —N(C₁-C₁₀ alkyl)₂; —O—(C₂-C₁₀ alkenyl), —S—(C₂-C₁₀ alkenyl), —NH—(C₂-C₁₀ alkenyl), or —N(C₂-C₁₀ alkenyl)₂; —O—(C₂-C₁₀ alkynyl), —S—(C₂-C₁₀ alkynyl), —NH—(C₂-C₁₀ alkynyl), or —N(C₂-C₁₀ alkynyl)₂; or —O—(C₁-C₁₀ alkylene)-O—(C₁-C₁₀ alkyl), —O—(C₁-C₁₀ alkylene)-NH—(C₁-C₁₀ alkyl) or —O—(C₁-C₁₀ alkylene)-NH(C₁-C₁₀ alkyl)₂, —NH—(C₁-C₁₀ alkylene)-O—(C₁-C₁₀ alkyl), or —N(C₁-C₁₀ alkyl)-(C₁-C₁₀ alkylene)-O—(C₁-C₁₀ alkyl), wherein the alkyl, alkylene, alkenyl and alkynyl may be substituted or unsubstituted. Examples of substituents include, and are not limited to, —O(CH₂)ₙOCH₃, and —O(CH₂)ₙNH₂, wherein n is from 1 to about 10, MOE, DMAOE, DMAEOE. Also contemplated herein are modified sugars described in WO 2001/088198; and Martin et al., Helv. Chim. Acta, 1995, 78, 486-504. In some embodiments, a modified sugar comprises one or more groups selected from a substituted silyl group, an RNA cleaving group, a reporter group, a fluorescent label, an intercalator, a group for improving the pharmacokinetic properties of a nucleic acid, a group for improving the pharmacodynamic properties of a nucleic acid, or other substituents having similar properties. In some embodiments, modifications are made at one or more of the 2′, 3′, 4′, 5′, or 6′ positions of the sugar or modified sugar, including the 3′ position of the sugar on the 3′-terminal nucleotide or in the 5′ position of the 5′-terminal nucleotide.

In some embodiments, the 2′-OH of a ribose is replaced with a substituent including one of the following: —H, —F; —CF₃, —CN, —N₃, —NO, —NO₂, —OR′, —SR′, or —N(R′)₂, wherein each R′ is independently as defined above and described herein; —O—(C₁-C₁₀ alkyl), —S—(C₁-C₁₀ alkyl), —NH—(C₁-C₁₀ alkyl), or —N(C₁-C₁₀ alkyl)₂; —O—(C₂-C₁₀ alkenyl), —S—(C₂-C₁₀ alkenyl), —NH—(C₂-C₁₀ alkenyl), or —N(C₂-C₁₀ alkenyl)₂; —O—(C₂-C₁₀ alkynyl), —S—(C₂-C₁₀ alkynyl), —NH—(C₂-C₁₀ alkynyl), or —N(C₂-C₁₀ alkynyl)₂; or —O—(C₁-C₁₀ alkylene)-O—(C₁-C₁₀ alkyl), —O—(C₁-C₁₀ alkylene)-NH—(C₁-C₁₀ alkyl) or —O—(C₁-C₁₀ alkylene)-NH(C₁-C₁₀ alkyl)₂, —NH—(C₁-C₁₀ alkylene)-O—(C₁-C₁₀ alkyl), or —N(C₁-C₁₀ alkyl)-(C₁-C₁₀ alkylene)-O—(C₁-C₁₀ alkyl), wherein the alkyl, alkylene, alkenyl and alkynyl may be substituted or unsubstituted. In some embodiments, the 2′-OH is replaced with —H (deoxyribose). In some embodiments, the 2′-OH is replaced with —F. In some embodiments, the 2′-OH is replaced with —OR′. In some embodiments, the 2′-OH is replaced with —OMe. In some embodiments, the 2′-OH is replaced with —OCH₂CH₂OMe.

Modified sugars also include locked nucleic acids (LNAs). In some embodiments, the locked nucleic acid has the structure indicated below. A locked nucleic acid of the structure below is indicated, wherein Ba represents a nucleobase or modified nucleobase as described herein, and wherein R^{2s} is —OCH₂C4′-

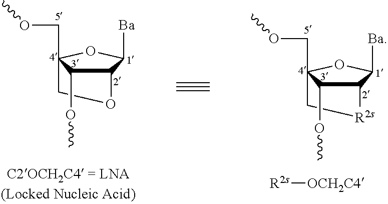

In some embodiments, a modified sugar is an ENA such as those described in, e.g., Seth et al., J Am Chem Soc. 2010 Oct. 27; 132 (42): 14942-14950. In some embodiments, a modified sugar is any of those found in an XNA (xenonucleic acid), for instance, arabinose, anhydrohexitol, threose, 2′fluoroarabinose, or cyclohexene.

Modified sugars include sugar mimetics such as cyclobutyl or cyclopentyl moieties in place of the pentofuranosyl sugar (see, e.g., U.S. Pat. Nos. 4,981,957; 5,118,800; 5,319,080; and 5,359,044). Some modified sugars that are contemplated include sugars in which the oxygen atom within the ribose ring is replaced by nitrogen, sulfur, selenium, or carbon. In some embodiments, a modified sugar is a modified ribose wherein the oxygen atom within the ribose ring is replaced with nitrogen, and wherein the nitrogen is optionally substituted with an alkyl group (e.g., methyl, ethyl, isopropyl, etc.).

Non-limiting examples of modified sugars include glycerol, which form glycerol nucleic acid (GNA) analogues. One example of a GNA analogue is described in Zhang, R et al., J. Am. Chem. Soc., 2008, 130, 5846-5847; Zhang L, et al., J. Am. Chem. Soc., 2005, 127, 4174-4175 and Tsai C H et al., PNAS, 2007, 14598-14603. Another example of a GNA derived analogue, flexible nucleic acid (FNA) based on the mixed acetal aminal of formyl glycerol, is described in Joyce G F et al., PNAS, 1987, 84, 4398-4402 and Heuberger B D and Switzer C, J. Am. Chem. Soc., 2008, 130, 412-413. Additional non-limiting examples of modified sugars include hexopyranosyl (6′ to 4′), pentopyranosyl (4′ to 2′), pentopyranosyl (4′ to 3′), or tetrofuranosyl (3′ to 2′) sugars.

Modified sugars and sugar mimetics can be prepared by methods known in the art, including, but not limited to: A. Eschenmoser, Science (1999), 284:2118; M. Bohringer et al, Helv. Chim. Acta (1992), 75:1416-1477; M. Egli et al, J. Am. Chem. Soc. (2006), 128 (33): 10847-56; A. Eschenmoser in Chemical Synthesis: Gnosis to Prognosis, C. Chatgilialoglu and V. Sniekus, Ed., (Kluwer Academic, Netherlands, 1996), p. 293; K.-U. Schoning et al, Science (2000), 290:1347-1351; A. Eschenmoser et al, Helv. Chim. Acta (1992), 75:218; J. Hunziker et al, Helv. Chim. Acta (1993), 76:259; G. Otting et al, Helv. Chim. Acta (1993), 76:2701; K. Groebke et al, Helv. Chim. Acta (1998), 81:375; and A. Eschenmoser, Science (1999), 284:2118. Modifications to the 2′ modifications can be found in Verma, S. et al. Annu. Rev. Biochem. 1998, 67, 99-134 and all references therein. Specific modifications to the ribose can be found in the following references: 2′-fluoro (Kawasaki et. al., J. Med. Chem., 1993, 36, 831-841), 2′-MOE (Martin, P. Helv. Chim. Acta 1996, 79, 1930-1938), “LNA” (Wengel, J. Acc. Chem. Res. 1999, 32, 301-310); PCT Publication No. WO2012/030683.

According to certain embodiments, various nucleotide modifications or nucleotide modification patterns may be used selectively in either the sense or antisense strand of an inhibitory RNA (e.g., siRNA) described herein. For example, in some embodiments one may utilize unmodified ribonucleotides in the antisense strand (at least within the duplex portion thereof) while employing modified nucleotides and/or modified or unmodified deoxyribonucleotides at some or all positions in the sense strand. In some embodiments, particular patterns of modifications are employed throughout part or all of either or both strands of an siRNA. Nucleotide modifications may occur in any of a variety of patterns. For example, an alternating pattern may be used. For example, the antisense, sense strand, or both, may have 2′-O-methyl or 2′-fluoro modifications on every other nucleotide. In some embodiments, an inhibitory RNA (e.g., siRNA) comprises a sense and/or antisense strand with at least one unmodified nucleotide.

In some embodiments, a sense and/or antisense strand comprises one or more motifs of three identical modifications on three consecutive nucleotides. For example, in some embodiments a double-stranded siRNA comprises one or more motifs of three identical modifications on three consecutive nucleotides in a sense strand, antisense strand, or both. In some embodiments, such a motif may occur at or near the cleavage site in either or both strands. Examples of such motifs are described in US Pat. App. Pubs. 2015/0197746, 2015/0247143, and 2016/0298124.

In some embodiments, an inhibitory RNA (e.g., siRNA) is a bluntmer of 19 nucleotides in length, wherein the sense strand contains at least one motif of three 2′-F modifications on three consecutive nucleotides at positions 7, 8, 9 from the 5′end, and where the antisense strand contains at least one motif of three 2′-O-methyl modifications on three consecutive nucleotides at positions 11, 12, 13 from the 5′end. In some embodiments, an inhibitory RNA (e.g., siRNA) is a double ended bluntmer of 20 nucleotides in length, wherein the sense strand contains at least one motif of three 2′-F modifications on three consecutive nucleotides at positions 8, 9, 10 from the 5′end, and where the antisense strand contains at least one motif of three 2′-O-methyl modifications on three consecutive nucleotides at positions 11, 12, 13 from the 5′end. In some embodiments, an inhibitory RNA (e.g., siRNA) is a double ended bluntmer of 21 nucleotides in length, wherein the sense strand contains at least one motif of three 2′-F modifications on three consecutive nucleotides at positions 9, 10, 11 from the 5′end, and where the antisense strand contains at least one motif of three 2′-O-methyl modifications on three consecutive nucleotides at positions 11, 12, 13 from the 5′end.

In some embodiments, an inhibitory RNA (e.g., siRNA) comprises a 19 nucleotide sense strand and a 21 nucleotide antisense strand, wherein the sense strand contains at least one motif of three 2′-F modifications on three consecutive nucleotides at positions 7, 8, 9 from the 5′end; the antisense strand contains at least one motif of three 2′-O-methyl modifications on three consecutive nucleotides at positions 11, 12, 13 from the 5′end, wherein one end of the inhibitory RNA (e.g., siRNA) is blunt, while the other end comprises a 2 nucleotide overhang. Preferably, the 2 nucleotide overhang is at the 3′-end of the antisense strand. When the 2 nucleotide overhang is at the 3′-end of the antisense strand, there may be two phosphorothioate internucleotide linkages between the terminal three nucleotides, wherein two of the three nucleotides are the overhang nucleotides, and the third nucleotide is a paired nucleotide next to the overhang nucleotide. In some embodiments, the inhibitory RNA (e.g., siRNA) additionally has two phosphorothioate internucleotide linkages between the terminal three nucleotides at both the 5 ‘-end of the sense strand and at the 5’-end of the antisense strand. In some embodiments, every nucleotide in the sense strand and the antisense strand of an inhibitory RNA (e.g., siRNA), including the nucleotides that are part of the motifs are modified nucleotides. In some embodiments each residue is independently modified with a 2′-O-methyl or 3′-fluoro, e.g., in an alternating motif.

In some embodiments, an inhibitory RNA (e.g., siRNA) comprises a 19 nucleotide sense strand and a 21 nucleotide antisense strand, wherein (i) the sense strand contains 2′-F modifications at positions 3, 7, 8, 9, 12, and 17 from the 5′end; (ii) the sense strand contains 2′-O-methyl modifications at positions 1, 2, 4, 5, 6, 10, 11, 13, 14, 15, 16, 18, and 19 from the 5′end; (iii) the antisense strand contains 2′-F modifications at positions 2 and 14 from the 5′end; and (iv) the antisense strand contains 2′-O-methyl modifications at positions 1, 3, 4, 5, 6, 7, 8, 9, 10, 11, 12, 13, 15, 16, 17, 18, 19, 20, and 21 from the 5′end; wherein one end of the inhibitory RNA (e.g., siRNA) is blunt, while the other end comprises a 2 nucleotide overhang at the 3′-end of the antisense strand. In some embodiments, the inhibitory RNA (e.g., siRNA) includes an antisense strand comprising two phosphorothioate internucleotide linkages between the terminal three nucleotides at the 3′ end, wherein two of the three nucleotides are the overhang nucleotides, and the third nucleotide is a paired nucleotide next to the overhang nucleotide. In some embodiments, an inhibitory RNA (e.g., siRNA) additionally has two phosphorothioate internucleotide linkages between the terminal three nucleotides at both the 5′-end of the sense strand and at the 5′-end of the antisense strand. In some embodiments, an inhibitory RNA (e.g., siRNA) has two phosphorothioate internucleotide linkages between the terminal three nucleotides at the 5′-end and/or the 3′-end of the sense strand and at the 5′-end and/or the 3′-end of the antisense strand.

In some embodiments, every nucleotide in the sense strand and antisense strand of an inhibitory RNA (e.g., siRNA), including the nucleotides that are part of the motifs, may be modified. Each nucleotide may be modified with the same or different modification which can include: one or more alteration of one or both of the non-linking phosphate oxygens and/or of one or more of the linking phosphate oxygens; alteration of a constituent of the ribose sugar, e.g., of the 2′ hydroxyl on the ribose sugar; wholesale replacement of the phosphate moiety with “dephospho” linkers; modification or replacement of a naturally occurring base; and replacement or modification of the ribose-phosphate backbone.

In some embodiments at least 50%, 60%, 70%, 80%, 90%, or more, e.g., 100% of the residues of the sense strand and antisense strand is independently modified with LNA, CRN, CET, UNA, HNA (1,5-anhydrohexitol nucleic acid), CeNA (cyclohexenyl nucleic acid—a DNA mimic in which the deoxyribose is replaced by a six-membered cyclohexene ring), 2′-methoxyethyl, 2′-O-methyl, 2′-O-allyl, 2′-C-allyl, 2′-deoxy, 2′-hydroxyl, or 2′-fluoro. The strands can contain more than one modification. In some embodiments at least 50%, 60%, 70%, 80%, 90%, or more, e.g., 100% of the residues of the sense strand and antisense strand is independently modified with 2′-O-methyl or 2′-fluoro. In some embodiments, at least two different modifications are present on the sense strand and antisense strand. Those two modifications may be the 2′-O-methyl or 2′-fluoro modifications, or others.

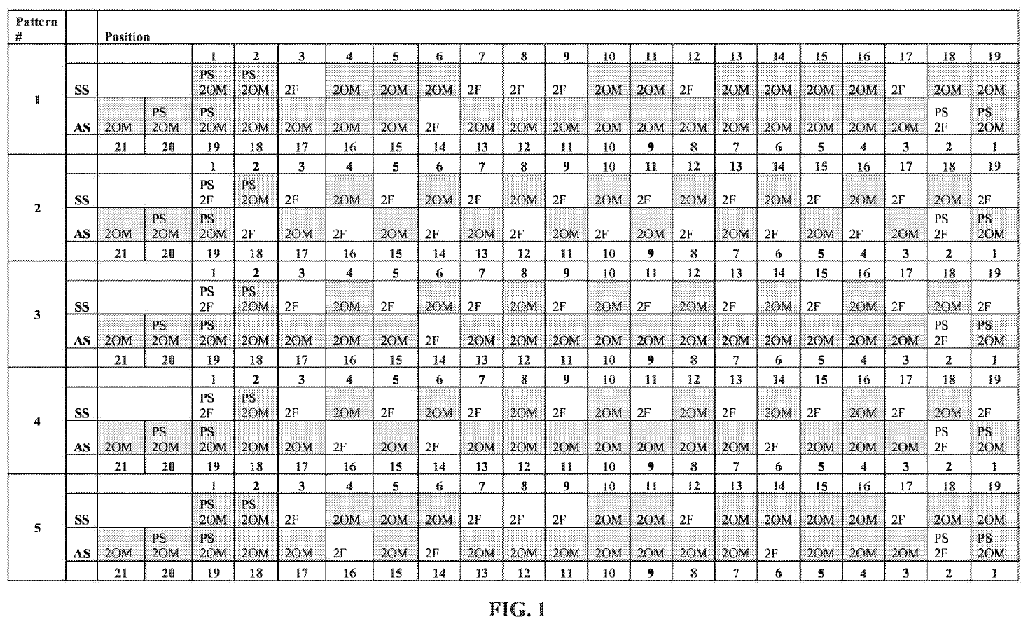

In some embodiments, the sense and antisense strands of the duplex of an inhibitory RNA (e.g., an siRNA), comprise any one of the modification patterns depicted as patterns 1-5 in FIG. 1. In FIG. 1, at any given position, a “2OM” represents a 2′-O-methyl modification and a “2F” represents a 2′-Fluoro modification. A “PS” represents a phosphorothioate bond between the nucleotide at a position noted with a “PS” and the adjacent nucleotide that is 3′ to the position noted with a “PS”. In some embodiments, any one of the antisense strands disclosed in SEQ ID NOs: 100-123 and 148-219, can be modified according to any one of the modification patterns 1-5 of the antisense strand (“AS”) disclosed in FIG. 1. In some embodiments, any one of the sense strands disclosed in SEQ ID NOs: 124-147, can be modified according to any one of the modification patterns 1-5 of the sense strand (“SS”) disclosed in FIG. 1. In some embodiments, the sense and/or antisense strands of the duplex of an inhibitory RNA (e.g., an siRNA), comprises any one of the modification patterns depicted as patterns 1-5 in FIG. 1, but where any 1, 2, 3, or 4 positions of the sense and/or antisense strands do not include the modification depicted at such 1, 2, 3, or 4 positions in one of patterns 1-5.

In some embodiments, an siRNA comprises any one of the modification patterns 1-5 (depicted in FIG. 1), and also includes a phosphorothioate bond between the last two, three, or four nucleotides of (i) the 5′ terminus of the sense strand; (ii) the 3′ terminus of the sense strand; (iii) the 5′ terminus of the antisense strand, and/or (iv) the 3′ terminus of the antisense strand. For example, in some embodiments, an siRNA includes (i) a sense strand that includes a phosphorothioate bond between the nucleotides at positions 1 and 2 from the 5′ end, and between the nucleotides at positions 2 and 3 from the 5′ end; (ii) a sense strand that includes a phosphorothioate bond between the nucleotides at positions 1 and 2 from the 3′ end, and between the nucleotides at positions 2 and 3 from the 3′ end; (iii) an antisense strand that includes a phosphorothioate bond between the nucleotides at positions 1 and 2 from the 5′ end, and between the nucleotides at positions 2 and 3 from the 5′ end; and/or (iv) an antisense strand that includes a phosphorothioate bond between the nucleotides at positions 1 and 2 from the 3′ end, and between the nucleotides at positions 2 and 3 from the 3′ end.

In some embodiments, an siRNA can be modified according to any one of the modification patterns 1-5 in FIG. 1 and can also be be conjugated to a ligand, e.g., as described herein. In some such cases, a ligand can be attached to any of the 3′ or 5′ terminus of the sense or antisense strand. In some embodiments, an siRNA (e.g., any of the siRNAs: 1-24 listed in Table 9) comprises a ligand (e.g., a GalNAc ligand, e.g., a GalNAc of Formula XD or XE described herein) conjugated to a terminus (e.g., 3′ or 5′ terminus of a sense or antisense strand), and said siRNA does not include a phosphorothioate bond between the two, three, or four nucleotides at the end of terminus that is conjugated to a ligand. For example, in some embodiments, an siRNA (e.g., any of the siRNAs: 1-24 listed in Table 9) includes a ligand (e.g., a GalNAc ligand, e.g., a GalNAc of Formula XD or XE described herein) conjugated to the 5′ end of the sense strand, and the siRNA includes (i) a sense strand that does not includes a phosphorothioate bond between the nucleotides at positions 1, 2, 3, or 4 from the 5′ end; (ii) a sense strand that includes a phosphorothioate bond between the nucleotides at positions 1 and 2 from the 3′ end, and between the nucleotides at positions 2 and 3 from the 3′ end; (iii) an antisense strand that includes a phosphorothioate bond between the nucleotides at positions 1 and 2 from the 5′ end, and between the nucleotides at positions 2 and 3 from the 5′ end; and (iv) an antisense strand that includes a phosphorothioate bond between the nucleotides at positions 1 and 2 from the 3′ end, and between the nucleotides at positions 2 and 3 from the 3′ end.

In some embodiments, an siRNA (e.g., any of the siRNAs: 1-24 listed in Table 9) includes a ligand (e.g., a GalNAc ligand, e.g., a GalNAc of Formula XD or XE described herein) conjugated to the 3′ end of the sense strand, and the siRNA includes (i) a sense strand that includes a phosphorothioate bond between the nucleotides at positions 1 and 2 from the 5′ end, and between the nucleotides at positions 2 and 3 from the 5′ end; (ii) a sense strand that does not include a phosphorothioate bond between the nucleotides at positions 1, 2, 3, or 4 from the 3′ end; (iii) an antisense strand that includes a phosphorothioate bond between the nucleotides at positions 1 and 2 from the 5′ end, and between the nucleotides at positions 2 and 3 from the 5′ end; and (iv) an antisense strand that includes a phosphorothioate bond between the nucleotides at positions 1 and 2 from the 3′ end, and between the nucleotides at positions 2 and 3 from the 3′ end.

In some embodiments, an siRNA (e.g., any of the siRNAs: 1-24 listed in Table 9) includes a ligand (e.g., a GalNAc ligand, e.g., a GalNAc of Formula XD or XE described herein) conjugated to the 5′ end of the antisense strand, and the siRNA includes (i) a sense strand that includes a phosphorothioate bond between the nucleotides at positions 1 and 2 from the 5′ end, and between the nucleotides at positions 2 and 3 from the 5′ end; (ii) a sense strand that includes a phosphorothioate bond between the nucleotides at positions 1 and 2 from the 3′ end, and between the nucleotides at positions 2 and 3 from the 3′ end; (iii) an antisense strand that does not include a phosphorothioate bond between the nucleotides at positions 1, 2, 3, or 4 from the 5′ end; and (iv) an antisense strand that includes a phosphorothioate bond between the nucleotides at positions 1 and 2 from the 3′ end, and between the nucleotides at positions 2 and 3 from the 3′ end.

In some embodiments, an siRNA (e.g., any of the siRNAs: 1-24 listed in Table 9) includes a ligand (e.g., a GalNAc ligand, e.g., a GalNAc of Formula XD or XE described herein) conjugated to the 3′ end of the antisense strand, and the siRNA includes (i) a sense strand that includes a phosphorothioate bond between the nucleotides at positions 1 and 2 from the 5′ end, and between the nucleotides at positions 2 and 3 from the 5′ end; (ii) a sense strand that includes a phosphorothioate bond between the nucleotides at positions 1 and 2 from the 3′ end, and between the nucleotides at positions 2 and 3 from the 3′ end; (iii) an antisense strand that includes a phosphorothioate bond between the nucleotides at positions 1 and 2 from the 5′ end, and between the nucleotides at positions 2 and 3 from the 5′ end; and (iv) an antisense strand that does not include a phosphorothioate bond between the nucleotides at positions 1, 2, 3, or 4 from the 3′ end.

In some embodiments, an siRNA (e.g., any of the siRNAs: 1-24 listed in Table 9) comprises a ligand (e.g., a GalNAc ligand, e.g., a GalNAc of Formula XD or XE described herein) conjugated to a terminus (e.g., 3′ or 5′ terminus of a sense or antisense strand), and said siRNA includes a phosphorothioate bond between the two, three, or four nucleotides at the end of terminus that is conjugated to a ligand.

For example, in some embodiments, an siRNA (e.g., any of the siRNAs: 1-24 listed in Table 9) includes a ligand (e.g., a GalNAc ligand, e.g., a GalNAc of Formula XD or XE described herein) conjugated to the 5′ end of the sense strand, and the siRNA includes (i) a sense strand that includes a phosphorothioate bond between the nucleotides at positions 1, 2, 3, or 4 from the 5′ end; (ii) a sense strand that includes a phosphorothioate bond between the nucleotides at positions 1 and 2 from the 3′ end, and between the nucleotides at positions 2 and 3 from the 3′ end; (iii) an antisense strand that includes a phosphorothioate bond between the nucleotides at positions 1 and 2 from the 5′ end, and between the nucleotides at positions 2 and 3 from the 5′ end; and (iv) an antisense strand that includes a phosphorothioate bond between the nucleotides at positions 1 and 2 from the 3′ end, and between the nucleotides at positions 2 and 3 from the 3′ end.

In some embodiments, an siRNA (e.g., any of the siRNAs: 1-24 listed in Table 9) includes a ligand (e.g., a GalNAc ligand, e.g., a GalNAc of Formula XD or XE described herein) conjugated to the 3′ end of the sense strand, and the siRNA includes (i) a sense strand that includes a phosphorothioate bond between the nucleotides at positions 1 and 2 from the 5′ end, and between the nucleotides at positions 2 and 3 from the 5′ end; (ii) a sense strand that includes a phosphorothioate bond between the nucleotides at positions 1, 2, 3, or 4 from the 3′ end; (iii) an antisense strand that includes a phosphorothioate bond between the nucleotides at positions 1 and 2 from the 5′ end, and between the nucleotides at positions 2 and 3 from the 5′ end; and (iv) an antisense strand that includes a phosphorothioate bond between the nucleotides at positions 1 and 2 from the 3′ end, and between the nucleotides at positions 2 and 3 from the 3′ end.

In some embodiments, an siRNA (e.g., any of the siRNAs: 1-24 listed in Table 9) includes a ligand (e.g., a GalNAc ligand, e.g., a GalNAc of Formula XD or XE described herein) conjugated to the 5′ end of the antisense strand, and the siRNA includes (i) a sense strand that includes a phosphorothioate bond between the nucleotides at positions 1 and 2 from the 5′ end, and between the nucleotides at positions 2 and 3 from the 5′ end; (ii) a sense strand that includes a phosphorothioate bond between the nucleotides at positions 1 and 2 from the 3′ end, and between the nucleotides at positions 2 and 3 from the 3′ end; (iii) an antisense strand that includes a phosphorothioate bond between the nucleotides at positions 1, 2, 3, or 4 from the 5′ end; and (iv) an antisense strand that includes a phosphorothioate bond between the nucleotides at positions 1 and 2 from the 3′ end, and between the nucleotides at positions 2 and 3 from the 3′ end.

In some embodiments, an siRNA (e.g., any of the siRNAs: 1-24 listed in Table 9) includes a ligand (e.g., a GalNAc ligand, e.g., a GalNAc of Formula XD or XE described herein) conjugated to the 3′ end of the antisense strand, and the siRNA includes (i) a sense strand that includes a phosphorothioate bond between the nucleotides at positions 1 and 2 from the 5′ end, and between the nucleotides at positions 2 and 3 from the 5′ end; (ii) a sense strand that includes a phosphorothioate bond between the nucleotides at positions 1 and 2 from the 3′ end, and between the nucleotides at positions 2 and 3 from the 3′ end; (iii) an antisense strand that includes a phosphorothioate bond between the nucleotides at positions 1 and 2 from the 5′ end, and between the nucleotides at positions 2 and 3 from the 5′ end; and (iv) an antisense strand that includes a phosphorothioate bond between the nucleotides at positions 1, 2, 3, or 4 from the 3′ end.

In some embodiments, the sense and/or antisense strand comprises modifications of an alternating pattern. The term “alternating motif” as used herein refers to a motif having one or more modifications, each modification occurring on alternating groups of one or more nucleotides of one strand. For example, an alternating nucleotide may refer to one per every other nucleotide or one per every three nucleotides, or a similar pattern. For example, if A, B and C each represent one type of modification to the nucleotide, the alternating motif can be “ABABABABABAB . . . ,” “AABBAABBAABB . . . ,” “AABAABAABAAB . . . ,” “AAABAAABAAAB . . . ”. “AAABBBAAABBB . . . ,” or “ABCABCABCABC . . . ,” etc.

The type of modifications contained in the alternating motif may be the same or different. For example, if A, B, C, D each represent one type of modification on the nucleotide, the alternating pattern, i.e., modifications on every other nucleotide, may be the same, but each of the sense strand or antisense strand can be selected from several possibilities of modifications within the alternating motif such as “ABABAB . . . ”, “ACACAC . . . ” “BDBDBD . . . ” or “CDCDCD . . . ,” etc.

In some embodiments, an inhibitory RNA (e.g., siRNA) comprises the modification pattern for the alternating motif on the sense strand that is shifted relative to the modification pattern for the alternating motif on the antisense strand. The shift may be such that the modified group of nucleotides of the sense strand corresponds to a differently modified group of nucleotides of the antisense strand and vice versa. For example, when paired with the antisense strand in the dsRNA duplex, the alternating motif in the sense strand may start with “ABABAB” from 5 ‘-3’ of the strand and the alternating motif in the antisense strand may start with “BAB ABA” from 5 ‘-3 of the strand, within the duplex portion. As another example, the alternating motif in the sense strand may start with “AABBAABB” from 5’-3′ of the strand and the alternating motif in the antisense strand may start with “BBAABBAA” from 5 ‘-3’ of the strand, within the duplex portion, so that there is a complete or partial shift of the modification patterns between the sense strand and the antisense strand.

In some embodiments, an inhibitory RNA (e.g., siRNA) comprises the pattern of the alternating motif of 2′-O-methyl modification and 2′-F modification on the sense strand has a shift relative to the pattern of the alternating motif of 2′-O-methyl modification and 2′-F modification on the antisense strand, i.e., the 2′-O-methyl modified nucleotide on the sense strand base pairs with a 2′-F modified nucleotide on the antisense strand and vice versa. The 1 position of the sense strand may start with the 2′-F modification, and the 1 position of the antisense strand may start with the 2′-O-methyl modification.

In some embodiments, one or more motifs of three identical modifications can be introduced on three consecutive nucleotides of the sense strand and/or antisense strand to interrupt the initial modification pattern present in the sense strand and/or antisense strand. In some embodiments, when the motif of three identical modifications on three consecutive nucleotides is introduced to any of the strands, the modification of the nucleotide next to the motif is a different modification than the modification of the motif. For example, the portion of the sequence containing the motif is “ . . . . NaYYYNb . . . ,” where “Y” represents the modification of the motif of three identical modifications on three consecutive nucleotide, and “Na” and “Nb” represent a modification to the nucleotide next to the motif “YYY” that is different than the modification of Y, and where Na and Nb can be the same or different modifications.

An inhibitory RNA (e.g., siRNA) may further comprise at least one phosphorothioate or methylphosphonate internucleotide linkage. In some embodiments, the internucleotide linkage modification may occur on every nucleotide on the sense strand and/or antisense strand; each internucleotide linkage modification may occur in an alternating pattern on the sense strand and/or antisense strand; or the sense strand or antisense strand may contain both internucleotide linkage modifications in an alternating pattern. The alternating pattern of the internucleotide linkage modification on the sense strand may be the same or different from the antisense strand, and the alternating pattern of the internucleotide linkage modification on the sense strand may have a shift relative to the alternating pattern of the internucleotide linkage modification on the antisense strand. In some embodiments, an inhibitory RNA (e.g., siRNA) comprises 6-8 phosphorothioate internucleotide linkages. In some embodiments, the antisense strand comprises two phosphorothioate internucleotide linkages at the 5′-terminus and two phosphorothioate internucleotide linkages at the 3′-terminus, and the sense strand comprises at least two phosphorothioate internucleotide linkages at either the 5′-terminus or the 3′-terminus.

In certain embodiments, an inhibitory RNA (e.g., siRNA) may have any of the configurations and/or modification patterns described from p. 59 (line 20) to p. 65 (line 15) of WO/2015/089368, or corresponding paragraphs [0469]-[0537] of US Pat. App. Pub. No. 2016/0298124 or in the claims of either or both of said publications. For example, in some embodiments an inhibitory RNA (e.g., siRNA) comprises a sense strand and an antisense strand, wherein said sense strand is complementary to said antisense strand, wherein said antisense strand comprises a region complementary to part of an mRNA encoding factor B (e.g., a target region described herein), wherein each strand is about 14 to about 30 nucleotides in length, wherein said agent is represented by formula (III):

sense:
5′ nₚ-Nₐ-(X X X)ᵢ- N_{b}- Y Y Y-N_{b}-(Z Z Z)ⱼ-Nₐ-
n_{q} 3′
antisense:
3′ n_{p′}-N_{a′}-(X′X′X′)ₖ-N_{b′}-Y′Y′Y′-N_{b′}-(Z′Z′Z′)ₗ-
N_{a′}-n_{q′} 5′

wherein: i, j, k, and l are each independently 0 or 1; p, p′, q, and q′ are each independently 0-6; each Nₐ and Nₐ′ independently represents an oligonucleotide sequence comprising 0-25 nucleotides which are either modified or unmodified or combinations thereof, each sequence comprising at least two differently modified nucleotides; each N_{b} and N_{b}′ independently represents an oligonucleotide sequence comprising 0-10 nucleotides which are either modified or unmodified or combinations thereof; each nₚ, nₚ′, n_{q}, and n_{q}′, each of which may or may not be present, independently represents an overhang nucleotide; XXX, YYY, ZZZ, X′X′X′, Y′Y′Y′, and Z′Z′Z′ each independently represent one motif of three identical modifications on three consecutive nucleotides; modifications on N_{b} differ from the modification on Y and modifications on N_{b}′ differ from the modification on Y′; and wherein the sense strand is conjugated to at least one ligand. In some embodiments i is 0; j is 0; i is 1; j is 1; both i and j are 0; or both i and j are 1. In some embodiments XXX is complementary to X′X′X′, YYY is complementary to Y′Y′Y′, and ZZZ is complementary to Z′Z′Z′. It should be understood that each X may comprise a different base, so long as each X comprises the same modification. For example, XXX could represent AGC where each nucleotide comprises a 2-F modification. Similarly, each X′, each Y, each Y′, each Z, and each Z may be different.

In some embodiments formula (III) is represented by formula (IIIa):

sense:
5′ nₚ-Nₐ- Y Y Y-Nₐ- n_{q} 3′
antisense:
3′ n_{p′}-N_{a′}-Y′Y′Y′-N_{a′}-n_{q′} 5′

or wherein formula (III) is represented by formula (IIIb):

sense:
5′ nₚ-Nₐ-Y Y Y-N_{b}-Z Z Z-Nₐ-n_{q} 3′
antisense:
3′ n_{p′}-N_{a′}-Y′Y′Y′-N_{b′}-Z′Z′Z′-N_{a′}-n_{q′} 5′

wherein each N_{b} and N_{b}′ independently represents an oligonucleotide sequence comprising 1-5 modified nucleotides; or wherein formula (III) is represented by formula (IIIc):

sense:
5′ nₚ-Nₐ- X X X-N_{b}-Y Y Y-Nₐ-n_{q} 3′
antisense:
3′ n_{p′}-N_{a′}-X′X′X′-N′_{b′}-Y′Y′Y′-N_{a′}-n_{q′} 5′

wherein each N_{b} and N_{b}′ independently represents an oligonucleotide sequence comprising 1-5 modified nucleotides; or wherein formula (III) is represented by formula (IIId):

sense:
5′ nₚ-Nₐ- X X X-N_{b}-Y Y Y- N_{b}-Z Z Z- Nₐ-n_{q} 3′
antisense:
3′ n_{p′}-N_{a′}-X′X′X′-N_{b′}-Y′Y′Y′-N_{b′}-Z′Z′Z′-N_{a′}-n_{q′} 5

wherein each N_{b} and N_{b}′ independently represents an oligonucleotide sequence comprising 1-5 modified nucleotides and each Nₐ and Nₐ′ independently represents an oligonucleotide sequence comprising 2-10 modified nucleotides.

In some embodiments, the modifications on the nucleotides are selected from the group consisting of LNA, CRN, CET, UNA, HNA, CeNA, 2′-methoxyethyl, 2′-O-methyl, 2′-O-alkyl, 2′-O-allyl, 2′-C-allyl, 2′-fluoro, 2′-deoxy, 2′-hydroxyl, and combinations thereof.

In some embodiments, the modifications on the nucleotides are 2′-O-methyl or 2′-fluoro modifications. In some embodiments the ligand is one or more GalNAc derivatives attached through a bivalent or trivalent branched linker. In some embodiments the ligand is depicted in Formula XA, XB, or XC, XD, XE, or another GalNAc structure shown below.

In some embodiments the ligand is attached to the 3′ end of the sense strand. In some embodiments the attachment is as depicted in Formula XD shown below. In some embodiments the attachment is as depicted in Formula XE shown below.

In some embodiments, an inhibitory RNA (e.g., siRNA) further comprises at least one phosphorothioate or methylphosphonate internucleotide linkage.

In some embodiments p′>0; or p′=2.

In some embodiments q′=0, p=0, q=0, and p′ overhang nucleotides are complementary to factor B mRNA. In some embodiments q′=0, p=0, q=0, and p′ overhang nucleotides are non-complementary to factor B mRNA.

In some embodiments at least one nₚ′ is linked to a neighboring nucleotide via a phosphorothioate linkage.

In some embodiments the ligand targets the nucleic acid molecule to hepatocytes. For example, in some embodiments the ligand binds to hepatocyte-specific asialoglycoprotein receptor (ASGPR), e.g., the ligand comprises a galactose derivative, e.g., GalNAc.

In some embodiments the ligand targets the nucleic acid molecule to the brain. For example, in some embodiments, the ligand is a rabies virus glycoprotein (RVG) peptide that facilitates delivery to the brain. In some embodiments, the ligand binds to transferrin receptor TfR or another target in the brain.

In some embodiments an inhibitory RNA (e.g., siRNA) is conjugated to or otherwise physically associated with one or more moieties that modulate, e.g., enhance, the activity, stability, cellular distribution, and/or cellular uptake of the inhibitory RNA (e.g., siRNA) and/or alter one or more physical properties of the inhibitory RNA (e.g., siRNA), such as charge or solubility. In some embodiments, a moiety may comprise an antibody or ligand. A ligand may be a carbohydrate, lectin, protein, glycoprotein, lipid, cholesterol, a fatty acid (e.g., docosahexaenoic acid (DHA)), steroid, bile acid, nucleic acid hormone, growth factor, or receptor. In some embodiments a biologically inactive variant of a naturally occurring hormone, growth factor, or other ligand may be used. In some embodiments, the moiety comprises a targeting moiety that targets the inhibitory RNA (e.g., siRNA) to a specified cell type, e.g., a hepatocyte. In some embodiments a targeting moiety binds to hepatocyte-specific asialoglycoprotein receptor (ASGPR).

In some embodiments a moiety is attached to an inhibitory RNA (e.g., siRNA) via a reversible linkage. A “reversible linkage” is a linkage that comprises a reversible bond. A “reversible bond” (also referred to as a labile bond or cleavable bond) is a covalent bond other than a covalent bond to a hydrogen atom that is capable of being selectively broken or cleaved more rapidly than other bonds in a molecule under selected conditions, the bond is capable of being selectively broken or cleaved under conditions that substantially will not break or cleave other covalent bonds in the same molecule. Cleavage or lability of a bond may be described in terms of the half-life (t_{1/2}) of bond cleavage (the time required for half of the bonds to cleave). Unless otherwise indicated, a reversible bond of interest herein is a “physiologically reversible bond”, by which is meant that the bond is cleavable under conditions normally encountered or analogous to those encountered within a mammalian body. A physiologically reversible linkage is a linkage that comprises at least one physiologically reversible bond. In some embodiments, a physiologically reversible bond is reversible under mammalian intracellular conditions, which include chemical conditions such as pH, temperature, oxidative or reductive conditions or agents, and salt concentration found in or analogous to those found in mammalian cells. Mammalian intracellular conditions also include the presence of enzymatic activity normally present in a mammalian cell, such as from proteolytic or hydrolytic enzymes. Enzymatically labile bonds are cleaved by enzymes in the body, e.g., intracellular enzymes. pH labile bonds are cleaved at a pH less than or equal to 7.0. Examples of reversible bonds and linkages and their use to conjugate moieties to an inhibitory RNA (e.g., siRNA) are described in, e.g., US Pat. App. Pub. Nos. 20130281685 and 20150273081.

In some embodiments, a moiety comprises a protein transduction domain (PTD). Protein transduction domains are polypeptides or portions thereof that facilitate uptake of heterologous molecules attached to the domain (such heterologous molecules may be referred to as “cargo”). A protein transduction domain that is a peptide may be referred to as a cell penetrating peptide (CPP)). A number of protein transduction domains/peptides are known in the art. PTDs include a variety of naturally occurring or synthetic arginine-rich peptides. An arginine-rich peptide is a peptide that contains at least 30% arginine residues, e.g., at least 40%, 50%, 60%, or more. Examples of PTDs include TAT (at least amino acids 49-56), Antennopedia homeodomain, HSV VP22, and polyarginine. Such peptides may be a cationic, hydrophobic, or amphipathic peptide and may include non-standard amino acids and/or various modifications or variations such as use of circularly permuted, inverso, retro, retro-inverso, or peptidomimetic versions. The attachment of a PTD and a cargo may be covalent or noncovalent.

Exemplary PTDs that may be used are described in U.S. Pat. App. Pub. Nos. 20090093026, 20090093425, 20120142763, 20150238516, and 20160215022. A PTD may comprise two or more PTDs (e.g., between 2 and 10 PTDs), which may be the same or different. PTDs may be directly linked to one another or may be separated by a linking portion that may comprise one or more amino acids and/or one or more non-amino acid moieties, such as an alkyl chain or oligoethylene glycol moiety.

In some embodiments, an inhibitory RNA (e.g., siRNA) comprises or is physically associated with an anionic charge neutralizing moiety. An anionic charge neutralizing moiety refers to a molecule or chemical group that can reduce the overall net anionic charge of a nucleic acid with which it is physically associated. One or more anionic charge neutralizing molecules or groups can be associated with a nucleic acid wherein each independently contributes to a reduction of the anionic charge and or increase in cationic charge. By charge neutralized is meant that the anionic charge of the nucleic acid is reduced, neutralized or more cationic than the same nucleic acid in the absence of an anionic charge neutralizing molecule or group. Phosphodiester and/or phosphothioate protecting groups are examples of anionic charge neutralizing groups. In some embodiments, an inhibitory RNA (e.g., siRNA) comprises a protecting group at one or more positions that reduces the net anionic charge of a backbone that contains negatively charged groups (e.g., a phosphodiester or phosphorothioate backbone). In some embodiments, the negatively charged phosphodiester backbone is neutralized by synthesis with bioreversible phosphotriester protecting groups that are converted into charged phosphodiester bonds inside cells by the action of cytoplasmic thioesterases, resulting in an agent that is biologically active for inhibiting expression, e.g., an inhibitory RNA (e.g., siRNA) that can mediate RNAi. Such agents, which are sometimes referred to as short interfering ribonucleic neutrals (siRNNs) can therefore serve as siRNA prodrugs. It should be understood that the backbone need not be completely neutralized (i.e., uncharged). In some embodiments, between 5% and 100% of the phosphate groups are protected, e.g., 25%-50% or 50% to 75% or 75% to 100%. In certain embodiments at least 5, 6, 7, 8, 9, or 10 of the phosphate groups on one or both strands are protected. Examples of useful phosphodiester and/or phosphothioate protecting groups, methods of making them, and their use in nucleic acids (e.g., to generate RNAi agent prodrugs) are described in US Pat. App. Pub. Nos. 2011/0294869, 2009/0093425, 2012/0142763, and 2015/0238516. In various embodiments a siRNA may comprise any of the modifications described herein. For example, in some embodiments it may contain 2′ sugar modifications (e.g., 2′-F, 2′-O-Me). Furthermore, a siRNN may have any of the configurations or modification patterns described herein.

In some embodiments a moiety attached to an inhibitory RNA (e.g., siRNA) comprises a carbohydrate. Representative carbohydrates include mono-, di-, tri- and oligosaccharides containing from about 4, 5, 6, 7, 8, or 9 monosaccharide units. In certain embodiments the carbohydrate comprises galactose or a galactose derivative such as galactosamine, N-formyl-galactosamine, N-acetylgalactosamine, N-propionyl-galactosamine, N-n-butanoyl-galactosamine, and N-iso-butanoylgalactos-amine. In certain embodiments of particular interest the galactose derivative comprises N-acetylgalactosamine (GalNAc). In certain embodiments, the moiety comprises multiple instances of the galactose or galactose derivative, e.g., multiple N-acetylgalactosamine moieties, e.g., 3 GalNAc moieties. As used herein, the term “galactose derivative” includes both galactose and derivatives of galactose having affinity for the asialoglycoprotein receptor equal to or greater than that of galactose. The term “galactose cluster” refers to a structure comprising at least 2 galactose derivatives that are physically associated with each other, typically by being covalently attached to another moiety. In some embodiments, a galactose cluster has 2-10 (e.g., 6), or 2-4 (e.g., 3) terminal galactose derivatives. A terminal galactose derivative may be attached to another moiety through the C-1 carbon of the galactose derivative. In some embodiments two or more, e.g., three, galactose derivatives are attached to a moiety that serves as a branch point and that can be attached to an inhibitory RNA (e.g., siRNA). In some embodiments, a galactose derivative is linked to the moiety that serves as a branch point via a linker or spacer. In some embodiments, the moiety that serves as a branch point may be attached to an inhibitory RNA (e.g., siRNA) via a linker or spacer. For example, in some embodiments, a galactose derivative is attached to a branch point via a linker or spacer that comprises an amide, carbonyl, alkyl, oligoethylene glycol moiety, or combination thereof. In some embodiments the linkers or spacers attached to each galactose derivative are the same. In some embodiments, a galactose cluster has three terminal galactosamines or galactosamine derivatives (e.g., GalNAc) each having affinity for the asialoglycoprotein receptor. A structure in which 3 terminal GalNAc moieties are attached (e.g., through the C-1 carbons of the saccharides) to a moiety that serves as branch point may be referred to as tri-antennary N-acetylgalactosamine (GalNAc₃). In some embodiments, one or more monomeric units comprising a galactose derivative may be incorporated site-specifically into an inhibitory RNA (e.g., siRNA). Such galactose derivative-containing monomeric units may comprise a galactose derivative, e.g., GalNAc, attached to a nucleoside or to a non-nucleoside moiety. In some embodiments, at least 3 nucleoside-GalNAc monomers or at least 3 non-nucleoside-GalNAc monomers are incorporated site-specifically into an inhibitory RNA (e.g., siRNA). In some embodiments, such incorporation may occur during solid-phase synthesis using phosphoramidite chemistry or via postsynthetic conjugation. In some embodiments, the galactose derivative-containing monomeric units are joined via phosphodiester bonds to each other and/or to nucleosides of the inhibitory RNA (e.g., siRNA) that do not have a galactose derivative attached. In some embodiments 2, 3, or more galactose derivative-containing monomeric units are arranged consecutively, i.e., without any intervening units that lack a galactose derivative. In some embodiments a carbohydrate, e.g., a galactose cluster, e.g., tri-antennary N-acetylgalactosamine or two or more GalNAc-containing monomeric units, is present at the end of a strand, e.g., at the 3′ end of the sense strand or at the 5′ end of an antisense strand. Exemplary carbohydrates (e.g., galactose clusters), galactose derivative-containing monomeric units, carbohydrate-modified inhibitory RNAs, and methods of manufacture and use thereof are described in US Pat. App. Pub. Nos. 2009/0203135, 2009/0239814, 2011/0207799, 2012/0157509, 2015/0247143, US Pub. '124; Nair, J K, et al., J. Am. Chem. Soc. 136, 16958-16961 (2014); Matsuda, S., et al., ACS Chem. Biol. 10, 1181-1187 (2015); Rajeev, K., et al., ChemBioChem 16, 903-908 (2015); Migawa, M T., et al., Bioorg Med Chem Lett. 26 (9): 2194-7 (2016); Prakash, T P, et al., J Med Chem. 59 (6): 2718-33 (2016). Exemplary galactose clusters are depicted below.

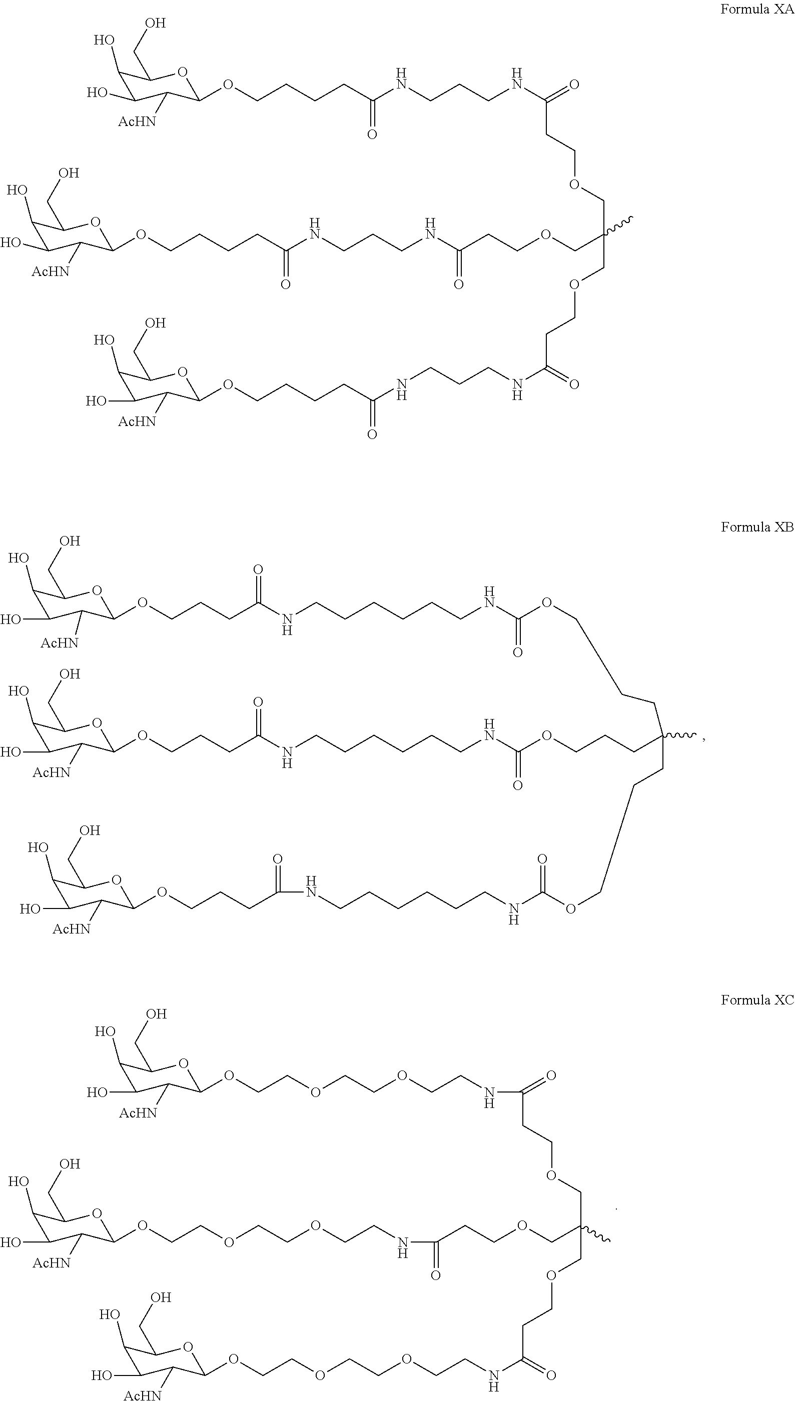

Additional GalNAc structures are depicted below (and can be synthesized as described in Sharma et al., Bioconjug. Chem. 29:2478-2488 (2018)):

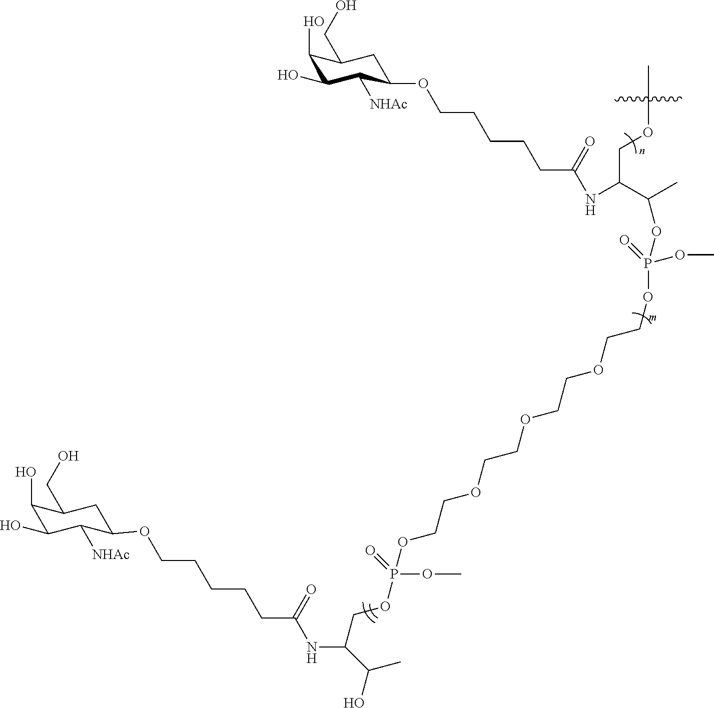

In some embodiments, m=0 and n=2. In some embodiments, m=1 and n=1. In some embodiments, m=1 and n=2. In some embodiments, m=1 and n=3.

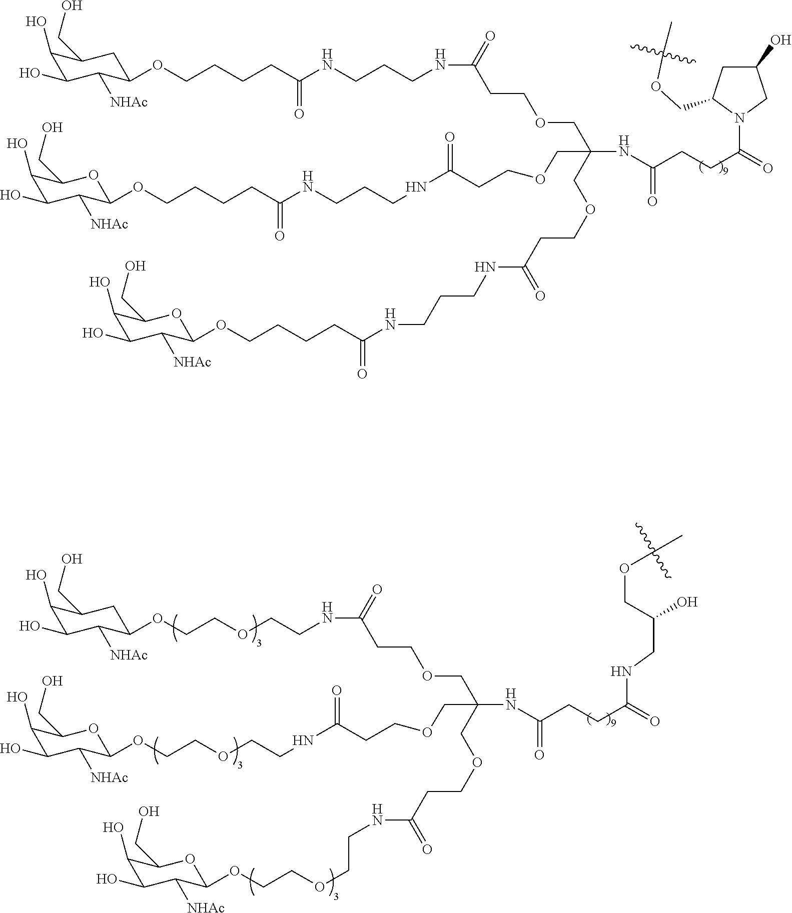

One of ordinary skill in the art appreciates that the structure of the linking moieties that connect each GalNAc to the branch point may vary. In some embodiments, an inhibitory RNA (e.g., siRNA) is conjugated to GalNAc as depicted below:

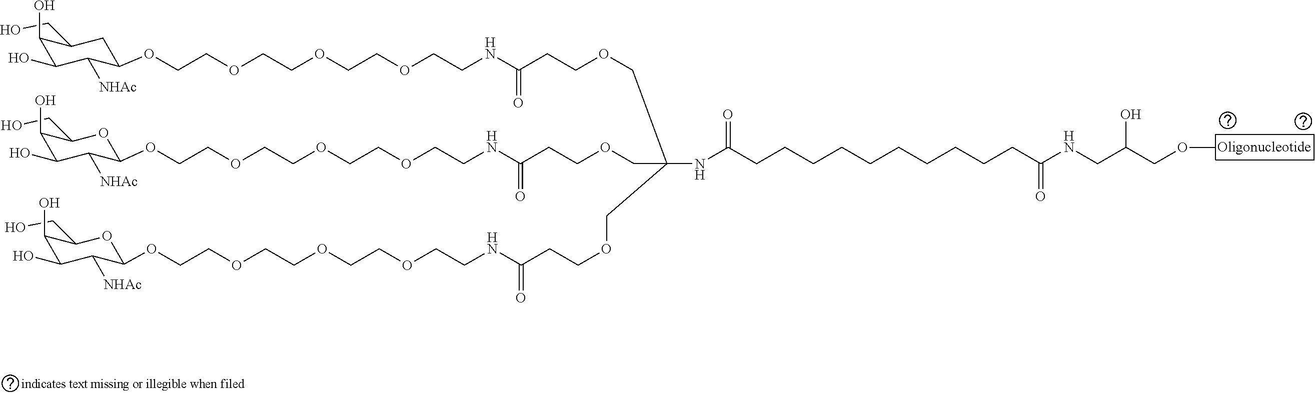 [SMILES: COCC(O)CNC(=O)CCCCCCCCCCC(=O)NC(COCCC(=O)NCCOCCOCCOCCOC1CC(CO)C(O)C(O)C1NC(C)=O)(COCCC(=O)NCCOCCOCCOCCOC1OC(CO)C(O)C(O)C1NC(C)=O)COCCC(=O)NCCOCCOCCOCCOC1OC(CO)C(O)C(O)C1NC(C)=O]

Formula XD (where said GalNAc can be conjugated at either strand (e.g., the sense strand) at the 3′ or 5′ end)

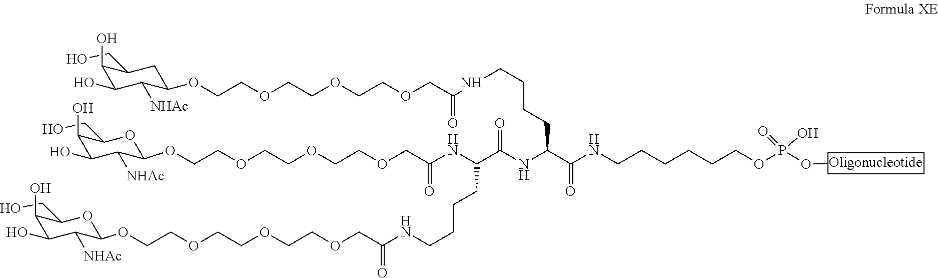

In some embodiments, an inhibitory RNA (e.g., any of the siRNAs: 1-24 listed in Table 9) is conjugated to a GalNAc ligand (e.g., a GalNAc of Formula XD or XE).

In some embodiments a GalNAc ligand (e.g., as shown in Formula XD or XE) is conjugated to the 3′-terminal nucleotide of the sense or antisense strand of an siRNA (e.g., any of the siRNAs: 1-24 listed in Table 9). In some embodiments, a GalNAc ligand (e.g., as shown in Formula XD or XE) is conjugated to the 3′ position of the sugar on the 3′-terminal nucleotide of the sense or antisense strand of an siRNA.

In some embodiments a GalNAc ligand (e.g., as shown in Formula XD or XE) is conjugated to the 5′-terminal nucleotide of the sense or antisense strand of an siRNA (e.g., any of the siRNAs: 1-24 listed in Table 9). In some embodiments a GalNAc ligand (e.g., as shown in Formula XD or XE) is conjugated to the 5′ position of the 5′-terminal nucleotide of the sense or antisense strand of an siRNA.

In some embodiments, when an inhibitory RNA (e.g., any of the siRNAs: 1-24 listed in Table 9) is conjugated to a ligand (e.g., a GalNAc ligand), the inhibitory RNA may not include a modification (e.g., a phosphorothioate bond “PS”) to the nucleotide(s) that is/are conjugated to the ligand.

In some embodiments, an siRNA (e.g., any of the siRNAs: 1-24 listed in Table 9) is conjugated to a GalNAc ligand (e.g., as shown in Formula XD or XE) at one terminus of either the sense or antisense strand. In some embodiments, the other three termini that are not conjugated to the GalNAc ligand contain a modification such as a phosphorothioate bond (“PS”). In some embodiments, a modification includes a PS bond between the two, three, or four 5′ or 3′-most nucleotides. In some embodiments, the terminus that is conjugated to a GalNAc ligand does not contain a phosphorothioate bond between the two, three or four 5′ or 3′-most nucleotides.

In some embodiments, an siRNA described herein can be conjugated to a galactose structure shown below:

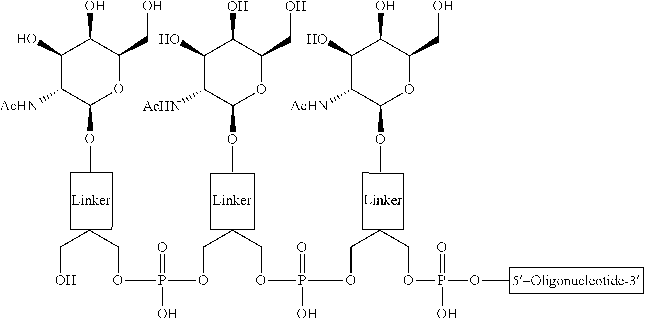

In some embodiments, the linker comprises an amide, carbonyl, alkyl, oligoethylene glycol moiety, or combination thereof.

In some embodiments, an siRNA described herein can be conjugated to a galactose structure shown below:

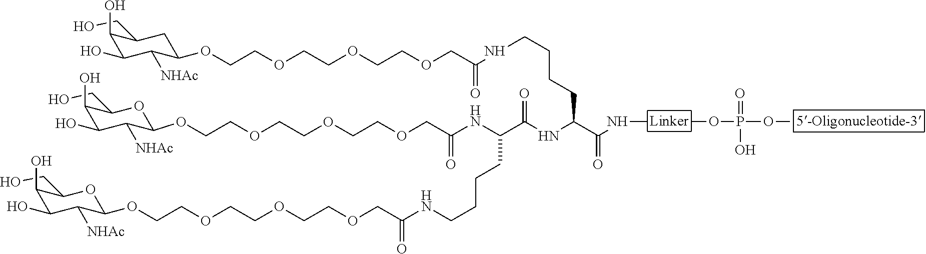

In some embodiments, the linker comprises an amide, carbonyl, alkyl, oligoethylene glycol moiety, or combination thereof.

Methods of synthesizing GalNAc ligands, methods of conjugating GalNAc ligands to inhibitory RNAs, and additional GalNAc ligands are known in the art and include, for example, those described in WO 2017/021385, WO 2017/178656, WO 2018/215391, WO 2019/145543, WO 2017/084987, WO 2017/055423, and WO 2012/083046, which are herein incorporated by reference in their entirety.

In some embodiments an inhibitory RNA (e.g., siRNA) is conjugated to a ligand as depicted below.

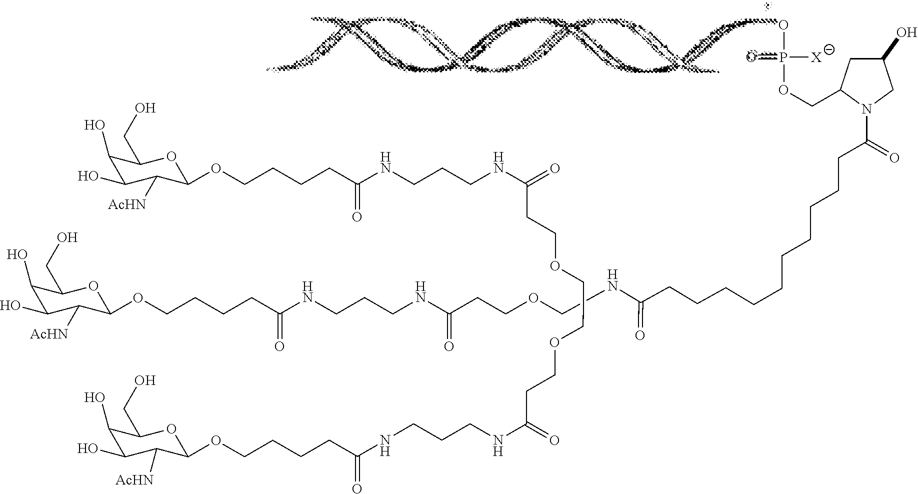

and, wherein X is O or S. In most embodiments, X is O. One of ordinary skill in the art will appreciate that the structure of the linking moiety that connects the galactose cluster to the phosphate group may vary.

In certain embodiments, the moiety comprises a lipophilic moiety. In some embodiments, the lipophilic moiety comprises a tocopherol, e.g., alpha-tocopherol. In some embodiments, the lipophilic moiety comprises cholesterol. In some embodiments, the lipophilic compound comprises an alkyl or heteroalkyl group. In some embodiments the lipophilic compound comprises palmitoyl, hexadec-8-enoyl, oleyl, (9E,12E)-octadeca-9,12-dienoyl, dioctanoyl, or C16-C20 acyl. In some embodiments, the lipophilic moiety comprises at least 16 carbon atoms. In some embodiments, the lipophilic moiety comprises —(CH)ₙ—NH—(C═O)—(CH)ₘ—CH₃. In some embodiments, n and m are each independently between 1 and 20. In some embodiments n+m is at least 10, 12, 14, or 16. In some embodiments, the lipophilic moiety is as shown below and/or is attached to a sugar moiety as shown below.

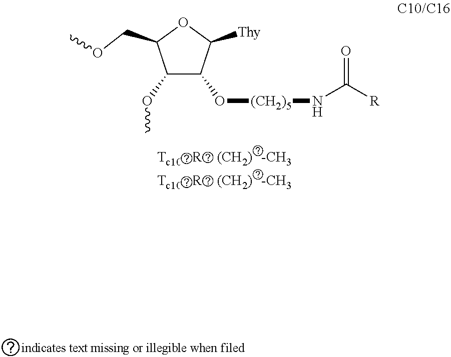

In general, a moiety may be attached at a terminus or internal subunit of an inhibitory RNA (e.g., siRNA). In some embodiments a moiety is attached to a modified subunit of the inhibitory RNA (e.g., siRNA). Those of ordinary skill in the art are aware of suitable methods to manufacture nucleic acids having moieties conjugated thereto. A nucleic acid strand comprising a modified nucleotide comprising a reactive functional group may be reacted with a moiety comprising a second reactive functional group, wherein the first and second reactive functional groups are capable of reacting with one another under conditions compatible with maintaining the structure of the nucleic acid strand. In some embodiments, a moiety may be attached to a sense strand or an antisense strand prior to hybridization of the strand with the complementary antisense or sense strand, respectively. In some embodiments, strands may be hybridized to form a duplex prior to incorporation of the moiety. In general, various methods of conjugation described herein may be used. See, e.g., Hermanson, G., Bioconjugate Techniques, 2nd ed., Academic Press, San Diego, 2008.

In some embodiments, an inhibitory RNA (e.g., siRNA) is a chimeric siRNA. “Chimeric” siRNAs as used herein, are siRNAs that contain two or more chemically distinct regions, each made up of at least one monomer unit, wherein the regions confer distinct properties on the compound. In some embodiments, at least one region is modified so as to confer upon the siRNA increased resistance to nuclease degradation, increased cellular uptake, and/or increased binding affinity for the target nucleic acid and at least one additional region of the siRNA can serve as a substrate for enzymes (e.g., RNase H) capable of cleaving RNA: DNA or RNA: RNA hybrids. In some embodiments, at least one region of the siRNA can serve as a substrate for enzymes (e.g., RNase H) capable of cleaving RNA: DNA or RNA: RNA hybrids and at least one region can inhibit translation by steric blocking.

In some embodiments, an inhibitory RNA (e.g., siRNA) described herein can be introduced to a target cell as an annealed duplex siRNA. In some embodiments, an inhibitory RNA (e.g., siRNA) described herein is introduced to a target cell as single stranded sense and antisense nucleic acid sequences that, once within the target cell, anneal to form an inhibitory RNA (e.g., siRNA) duplex. Alternatively, the sense and antisense strands of the inhibitory RNA (e.g., siRNA) can be encoded by an expression vector (such as an expression vector described herein) that is introduced to the target cell. Upon expression within the target cell, the transcribed sense and antisense strands can anneal to reconstitute the inhibitory RNA (e.g., SiRNA).

An inhibitory RNA (e.g., an siRNA or miRNA, or a vector comprising a nucleotide sequence encoding an siRNA or miRNA) described herein can be synthesized by standard methods known in the art, e.g., by use of an automated synthesizer. RNAs produced by such methodologies tend to be highly pure and to anneal efficiently to form inhibitory RNA (e.g., siRNA) duplexes. Following chemical synthesis, single stranded RNA molecules can be deprotected, annealed to form siRNAs, and purified (e.g., by gel electrophoresis or HPLC). Alternatively, standard procedures can be used for in vitro transcription of RNA from DNA templates, e.g., carrying one or more RNA polymerase promoter sequences (e.g., T7 or SP6 RNA polymerase promoter sequences). Protocols for preparation of siRNAs using T7 RNA polymerase are known in the art (see, e.g., Donze and Picard, Nucleic Acids Res. 2002; 30: e46; and Yu et al., Proc. Natl. Acad. Sci. USA 2002; 99:6047-6052). The sense and antisense transcripts can be synthesized in two independent reactions and annealed later, or they can be synthesized simultaneously in a single reaction.

An inhibitory RNA (e.g., an siRNA or miRNA) can also be formed within a cell by transcription of RNA from an expression construct introduced into the cell (see, e.g., Yu et al., Proc. Natl. Acad. Sci. USA 2002; 99:6047-6052). An expression construct for in vivo production of inhibitory RNA (e.g., siRNA) molecules can include one or more siRNA encoding sequences operably linked to elements necessary for the proper transcription of the siRNA encoding sequence(s), including, e.g., promoter elements and transcription termination signals. Preferred promoters for use in such expression constructs include the polymerase-III HI-RNA promoter (see, e.g., Brummelkamp et al., Science 2002; 296:550-553) and the U6 polymerase-III promoter (see, e.g., Sui et al., Proc. Natl. Acad. Sci. USA 2002; Paul et al., Nature Biotechnol. 2002; 20:505-508; and Yu et al., Proc. Natl. Acad. Sci. USA 2002; 99:6047-6052). An siRNA expression construct can further comprise one or more vector sequences that facilitate the cloning of the expression construct. Standard vectors that can be used include, e.g., pSilencer 2.0-U6 vector (Ambion Inc., Austin, Tex.).

### VI. Expression Vectors

In some embodiments, an inhibitory RNA described herein is delivered to a subject (e.g., to a cell of a subject, e.g., a liver cell of a subject) using an expression vector. Many forms of vectors can be used to deliver an inhibitory RNA described herein. Non-limiting examples of expression vectors include viral vectors (e.g., vectors suitable for gene therapy), plasmid vectors, bacteriophage vectors, cosmids, phagemids, artificial chromosomes, and the like.

In some embodiments, a nucleotide sequence encoding an inhibitory RNA described herein is integrated into a viral vector. Non-limiting examples of viral vectors include: retrovirus (e.g., Moloney murine leukemia virus (MMLV), Harvey murine sarcoma virus, murine mammary tumor virus, Rous sarcoma virus), adenovirus, adeno-associated virus, SV40-type virus, polyomavirus, Epstein-Barr virus, papilloma virus, herpes virus, vaccinia virus, and polio virus.

In vivo, many complement proteins, including factor B, are synthesized primarily in the liver. As such, in some embodiments, hepatocytes are targeted for delivery of an inhibitory RNA described herein. Several classes of viral vectors have been shown competent for liver-targeted delivery of a gene therapy construct, including retroviral vectors (see, e.g., Axelrod et al., PNAS 87:5173-5177 (1990); Kay et al., Hum. Gene Ther. 3:641-647 (1992); Van den Driessche et al., PNAS 96:10379-10384 (1999); Xu et al., ASAIO J. 49:407-416 (2003); and Xu et al., PNAS 102:6080-6085 (2005)), lentiviral vectors (see, e.g., Mckay et al., Curr. Pharm. Des. 17:2528-2541 (2011); Brown et al., Blood 109:2797-2805 (2007); and Matrai et al., Hepatology 53:1696-1707 (2011)), adeno-associated viral (AAV) vectors (see, e.g., Herzog et al., Blood 91:4600-4607 (1998)), and adenoviral vectors (see, e.g., Brown et al., Blood 103:804-810 (2004) and Ehrhardt et al., Blood 99:3923-3930 (2002)).

Retroviruses are enveloped viruses that belong to the viral family Retroviridae. Once in a host's cell, the virus replicates by using a viral reverse transcriptase enzyme to transcribe its RNA into DNA. The retroviral DNA replicates as part of the host genome, and is referred to as a provirus. A selected nucleic acid can be inserted into a vector and packaged in retroviral particles using techniques known in the art. Protocols for the production of replication-deficient retroviruses are known in the art (see, e.g., Kriegler, M., Gene Transfer and Expression, A Laboratory Manual, W.H. Freeman Co., New York (1990) and Murry, E. J., Methods in Molecular Biology, Vol. 7, Humana Press, Inc., Cliffton, N.J. (1991)). The recombinant virus can then be isolated and delivered to cells of the subject either in vivo or ex vivo. A number of retroviral systems are known in the art, for example See U.S. Pat Nos. 5,994, 136, 6, 165,782, and 6,428,953. Retroviruses include the genus of Alpharetrovirus (e.g., avian leukosis virus), the genus of Betaretrovirus; (e.g., mouse mammary tumor virus) the genus of Deltaretrovirus (e.g., bovine leukemia virus and human T-lymphotropic virus), the genus of Epsilonretrovirus (e.g., Walleye dermal sarcoma virus), and the genus of Lentivirus.

In some embodiments, the retrovirus is a lentivirus of the Retroviridae family. Lentiviral vectors can transduce non-proliferating cells and show low immunogenicity. In some examples, the lentivirus is, but is not limited to, human immunodeficiency viruses (HIV-1 and HIV-2), simian immunodeficiency virus (S1V), feline immunodeficiency virus (FIV), equine infections anemia (EIA), and visna virus. Vectors derived from lentiviruses can achieve significant levels of nucleic acid transfer in vivo.

In some embodiments, the vector is an adenovirus vector. Adenoviruses are a large family of viruses containing double stranded DNA. They replicate within the nucleus of a host cell, using the host's cell machinery to synthesize viral RNA, DNA and proteins. Adenoviruses are known in the art to affect both replicating and non-replicating cells, to accommodate large transgenes, and to code for proteins without integrating into the host cell genome.

In some embodiments, the viral vector is an adeno-associated virus (AAV) vector. AAV systems are generally well known in the art (see, e.g., Kelleher and Vos, Biotechniques, 17 (6): 1110-17 (1994); Cotten et al., P.N.A.S. U.S.A., 89 (13): 6094-98 (1992); Curiel, Nat Immun, 13 (2-3): 141-64 (1994); Muzyczka, Curr Top Microbiol Immunol, 158:97-129 (1992); and Asokan A, et al., Mol. Ther., 20 (4): 699-708 (2012)). Methods for generating and using recombinant AAV (rAAV) vectors are described, for example, in U.S. Pat. Nos. 5,139,941 and 4,797,368.

Several AAV serotypes have been characterized, including AAV1, AAV2, AAV3 (e.g., AAV3B), AAV4, AAV5, AAV6, AAV7, AAV8, AAV9, AAV10, and AAV11, as well as variants thereof. Generally, any AAV serotype may be used to deliver an inhibitory RNA described herein. However, the serotypes have different tropisms, e.g., they preferentially infect different tissues. In one embodiment, because complement proteins are produced in the liver, an AAV serotype is selected based on a liver tropism, found in at least serotypes AAV2, AAV3 (e.g., AAV3B), AAV5, AAV7, AAV8, and AAV9 (see, e.g., Shaoyong et al., Mol. Ther. 23:1867-1876 (2015)).

The AAV sequences of a rAAV vector typically comprise the cis-acting 5′ and 3′ inverted terminal repeat sequences (See, e.g., B. J. Carter, in “Handbook of Parvoviruses”, ed., P. Tijsser, CRC Press, pp. 155 168 (1990)). The ITR sequences are about 145 bp in length. In some embodiments, substantially the entire sequences encoding the ITRs are used in an rAAV vector, although some degree of minor modification of these sequences is permissible. The ability to modify these ITR sequences is within the skill of the art. (See, e.g., texts such as Sambrook et al, “Molecular Cloning. A Laboratory Manual”, 2d ed., Cold Spring Harbor Laboratory, New York (1989); and K. Fisher et al., J Virol., 70:520 532 (1996)). An example of an rAAV vector of the present disclosure is a “cis-acting” plasmid containing the transgene (e.g., nucleic acid encoding an inhibitory RNA described herein), in which the selected transgene sequence and associated regulatory elements are flanked by the 5′ and 3′ AAV ITR sequences. The AAV ITR sequences may be obtained from any known AAV, including presently identified mammalian AAV types.

In addition to the major elements identified above for an rAAV vector, the vector can also include conventional control elements operably linked to the transgene in a manner that permits its transcription, translation and/or expression in a cell transfected with the vector or infected with the virus produced by the disclosure. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A number of expression control sequences, including promoters that are native, constitutive, inducible and/or tissue-specific, are known in the art and may be included in a vector described herein. In some embodiments, operably linked coding sequences yield a functional RNA (e.g., miRNA or siRNA).

Examples of constitutive promoters include, without limitation, the retroviral Rous sarcoma virus (RSV) LTR promoter (optionally with the RSV enhancer), the cytomegalovirus (CMV) promoter (optionally with the CMV enhancer), the SV40 promoter, and the dihydrofolate reductase promoter. Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system, the ecdysone insect promoter, the tetracycline-repressible system, the tetracycline-inducible system, the RU486-inducible system and the rapamycin-inducible system. Still other types of inducible promoters which may be useful in this context are those which are regulated by a specific physiological state, e.g., temperature, acute phase, a particular differentiation state of the cell, or in replicating cells only. In another embodiment, a native promoter, or fragment thereof, for a transgene will be used. In a further embodiment, other native expression control elements, such as enhancer elements, polyadenylation sites or Kozak consensus sequences may also be used to mimic the native expression.

In some embodiments, regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers, etc.) are well known in the art. In some embodiments, the promoter is a chicken β-actin promoter, a pol II promoter, or a pol III promoter.

In some embodiments, an rAAV is designed for expressing an inhibitory RNA described herein in hepatocytes, and an rAAV includes one or more liver-specific regulatory elements, which substantially limit expression of the inhibitory RNA to hepatic cells. Generally, liver-specific regulatory elements can be derived from any gene known to be exclusively expressed in the liver. WO 2009/130208 identifies several genes expressed in a liver-specific fashion, including serpin peptidase inhibitor, clade A member 1, also known as α-antitrypsin (SERPINA1; GeneID 5265), apolipoprotein C-I (APOC1; GeneID 341), apolipoprotein C-IV (APOC4; GeneID 346), apolipoprotein H (APOH; GeneID 350), transthyretin (TTR; GeneID 7276), albumin (ALB; GeneID 213), aldolase B (ALDOB; GeneID 229), cytochrome P450, family 2, subfamily E, polypeptide 1 (CYP2E1; GeneID 1571), fibrinogen alpha chain (FGA; GeneID 2243), transferrin (TF; GeneID 7018), and haptoglobin related protein (HPR; GeneID 3250). In some embodiments, a viral vector described herein includes a liver-specific regulatory element derived from the genomic loci of one or more of these proteins. In some embodiments, a promoter may be the liver-specific promoter thyroxin binding globulin (TBG). Alternatively, other liver-specific promoters may be used (see, e.g., The Liver Specific Gene Promoter Database, Cold Spring Harbor, http://rulai.cshl.edu/LSPD/, such as, e.g., alpha 1 anti-trypsin (A1AT); human albumin (Miyatake et al., J. Virol. 71:5124 32 (1997)); humA1b; hepatitis B virus core promoter (Sandig et al., Gene Ther. 3:1002 9 (1996)); or LSP1. Additional vectors and regulatory elements are described in, e.g., Baruteau et al., J. Inherit. Metab. Dis. 40:497-517 (2017)).

In some embodiments, a viral vector (e.g., an rAAV vector) comprises a DNA sequence encoding an inhibitory RNA described herein.

In some embodiments, a vector (e.g., a viral vector) comprises one or more nucleotide sequences that encode more than one (e.g., 2, 3, 4, 5, or more) miRNAs or siRNAs comprising a nucleic acid strand that is complementary to a target portion of a factor B transcript, e.g., factor B mRNA (SEQ ID NO: 75). In some embodiments, a vector comprises multiple nucleotide sequences, where each nucleotide sequence encodes a different inhibitory RNA described herein. In some embodiments, a vector comprises multiple nucleotide sequences encoding at least 2 different inhibitory RNAs, wherein at least two of the nucleotide sequences are copies of the same inhibitory RNA described herein.

In some embodiments, in addition to one or more sequences encoding one or more inhibitory RNAs described herein, a vector (e.g., a viral vector) comprises one or more additional nucleotide sequences encoding one or more factor B inhibitors, e.g., a factor B inhibitor described herein. For example, a factor B inhibitor can be a polypeptide inhibitor and/or a nucleic acid aptamer (see, e.g., U.S. Publ. No. 20030191084). Exemplary polypeptide inhibitors include a compstatin analog (e.g., a compstatin analog described herein that includes genetically encodable amino acids), an anti-factor B antibody (e.g., scFv or single domain antibody, e.g., a nanobody), an enzyme that degrades factor B, or a mammalian complement regulatory protein (e.g., CR1, DAF, MCP, CFH, CFI, C1 inhibitor (C1-INH), or CFD inhibitors (e.g., lampalizumab or danicopan)), lectin pathway inhibitors (e.g., Narsopllimab), or a soluble form of complement receptor 1 (sCR1), TP10 or TP20 (Avant Therapeutics), or portion thereof. Additional polypeptide inhibitors include mini-factor H (see, e.g., U.S. Publ. No. 20150110766), Efb protein or complement inhibitor (SCIN) protein from Staphylococcus aureus, or a variant or derivative or mimetic thereof (see, e.g., U.S. Publ. 20140371133).

In some embodiments, a polypeptide inhibitor is linked to a secretion signal sequence for secretion of the expressed polypeptide inhibitor from a host cell.

### VII. Production of Expression Vectors

Methods for obtaining expression vectors, e.g., rAAVs, are known in the art. Typically, the methods involve culturing a host cell which contains a nucleic acid sequence encoding an AAV capsid protein or fragment thereof; a functional rep gene; a recombinant AAV vector composed of AAV inverted terminal repeats (ITRs) and a transgene; and/or sufficient helper functions to permit packaging of the recombinant AAV vector into the AAV capsid proteins.

The components to be cultured in a host cell to package a rAAV vector in an AAV capsid may be provided to the host cell in trans. Alternatively, any one or more of the required components (e.g., recombinant AAV vector, rep sequences, cap sequences, and/or helper functions) may be provided by a stable host cell that has been engineered to contain one or more of the required components using methods known to those of skill in the art. In some embodiments, such a stable host cell contains the required component(s) under the control of an inducible promoter. In other embodiments, the required component(s) may be under the control of a constitutive promoter. In other embodiments, a selected stable host cell may contain selected component(s) under the control of a constitutive promoter and other selected component(s) under the control of one or more inducible promoters. For example, a stable host cell may be generated that is derived from 293 cells (which contain E1 helper functions under the control of a constitutive promoter), but that contain the rep and/or cap proteins under the control of inducible promoters. Other stable host cells may be generated by one of skill in the art using routine methods.

Recombinant AAV vector, rep sequences, cap sequences, and helper functions required for producing an rAAV of the disclosure may be delivered to a packaging host cell using any appropriate genetic element (e.g., vector). A selected genetic element may be delivered by any suitable method known in the art, e.g., to those with skill in nucleic acid manipulation and include genetic engineering, recombinant engineering, and synthetic techniques (see, e.g., Sambrook et al, Molecular Cloning: A Laboratory Manual, Cold Spring Harbor Press, Cold Spring Harbor, N.Y.). Similarly, methods of generating rAAV virions are well known and any suitable method can be used with the present disclosure (see, e.g., K. Fisher et al, J. Virol., 70:520-532 (1993) and U.S. Pat. No. 5,478,745).

In some embodiments, recombinant AAVs may be produced using a triple transfection method (e.g., as described in U.S. Pat. No. 6,001,650). In some embodiments, recombinant AAVs are produced by transfecting a host cell with a recombinant AAV vector (comprising a transgene) to be packaged into AAV particles, an AAV helper function vector, and an accessory function vector. An AAV helper function vector encodes “AAV helper function” sequences (i.e., rep and cap), which function in trans for productive AAV replication and encapsidation. In some embodiments, the AAV helper function vector supports efficient AAV vector production without generating any detectable wild-type AAV virions (i.e., AAV virions containing functional rep and cap genes). Non-limiting examples of vectors suitable for use with the present disclosure include pHLP19 (see, e.g., U.S. Pat. No. 6,001,650) and pRep6cap6 vector (see, e.g., U.S. Pat. No. 6,156,303). An accessory function vector encodes nucleotide sequences for non-AAV derived viral and/or cellular functions upon which AAV is dependent for replication (i.e., “accessory functions”). Accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any known helper viruses such as adenovirus, herpesvirus (other than herpes simplex virus type-1), and vaccinia virus.

In some embodiments, the disclosure provides transfected host cells. The term “transfection” is used to refer to the uptake of foreign DNA by a cell, and a cell has been “transfected” when exogenous DNA has been introduced inside the cell membrane. A number of transfection techniques are generally known in the art (see, e.g., Graham et al. (1973) Virology, 52:456; Sambrook et al. (1989) Molecular Cloning, a laboratory manual, Cold Spring Harbor Laboratories, New York, Davis et al. (1986) Basic Methods in Molecular Biology, Elsevier; and Chu et al. (1981) Gene 13:197). Such techniques can be used to introduce one or more exogenous nucleic acids, such as a nucleotide integration vector and other nucleic acid molecules, into suitable host cells.

In some embodiments, a host cell is a mammalian cell. A host cell may be used as a recipient of an AAV helper construct, an AAV minigene plasmid, an accessory function vector, and/or other transfer DNA associated with the production of recombinant AAVs. The term includes the progeny of an original cell that has been transfected. Thus, a “host cell” as used herein may refer to a cell that has been transfected with an exogenous DNA sequence. It is understood that the progeny of a single parental cell may not necessarily be completely identical in morphology or in genomic or total DNA complement as the original parent, due to natural, accidental, or deliberate mutation.

Additional methods for generating and isolating AAV viral vectors suitable for delivery to a subject are described in, e.g., U.S. Pat. Nos. 7,790,449; 7,282,199; WO 2003/042397; WO 2005/033321, WO 2006/110689; and U.S. Pat. No. 7,588,772. In one system, a producer cell line is transiently transfected with a construct that encodes the transgene flanked by ITRs and a construct(s) that encodes rep and cap. In another system, a packaging cell line that stably supplies rep and cap is transiently transfected with a construct encoding the transgene flanked by ITRs. In each of these systems, AAV virions are produced in response to infection with helper adenovirus or herpesvirus, and rAAVs are separated from contaminating virus. Other systems do not require infection with helper virus to recover the AAV—the helper functions (i.e., adenovirus E1, E2a, VA, and E4 or herpesvirus UL5, UL8, UL52, and UL29, and herpesvirus polymerase) are also supplied, in trans, by the system. In such systems, helper functions can be supplied by transient transfection of the cells with constructs that encode the helper functions, or the cells can be engineered to stably contain genes encoding the helper functions, the expression of which can be controlled at the transcriptional or posttranscriptional level.

In yet another system, the transgene flanked by ITRs and rep/cap genes are introduced into insect host cells by infection with baculovirus-based vectors. Such production systems are known in the art (see generally, e.g., Zhang et al., 2009, Human Gene Therapy 20:922-929). Methods of making and using these and other AAV production systems are also described in U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065.

The foregoing methods for producing recombinant vectors are not meant to be limiting, and other suitable methods will be apparent to the skilled artisan.

### VIII. Compositions and Administration

Inhibitory RNAs (e.g., an siRNA or miRNA described herein), or a vector comprising a nucleotide sequence encoding an siRNA or miRNA described herein, can be used to treat a complement-mediated disease or disorder, e.g., subjects suffering from or susceptible to a complement-mediated disease or disorder described herein. The route and/or mode of administration of inhibitory RNAs described herein can vary depending upon the desired results. One with skill in the art, i.e., a physician, is aware that dosage regimens can be adjusted to provide the desired response, e.g., a therapeutic response. Methods of administration include, but are not limited to, intradermal, intramuscular, intraperitoneal, intravenous, subcutaneous, intranasal, epidural, oral, sublingual, intracerebral, intrathecal (e.g., intracisternal or via a lumbar puncture), intravaginal, transdermal, rectal, by inhalation, or topical, particularly to the ears, nose, eyes (e.g., intravitreal or suprachoroidal administration), or skin. In some embodiments, compositions of inhibitory RNAs are delivered to the central nervous system (CNS), e.g., delivered via intracerebroventricular administration. The mode of administration is left to the discretion of the practitioner.

One of skill in the art would understand that inhibitory RNAs (e.g., an siRNA or miRNA described herein), or a vector comprising a nucleotide sequence encoding an siRNA or miRNA described herein, may be delivered to the CNS (e.g., via intrathecal administration) to treat a disease or disorder affecting the CNS such as multiple sclerosis, Parkinson's disease, Huntington's disease, Alzheimer's disease, other chronic demyelinating diseases (e.g., neuromyelits optica), amyotrophic lateral sclerosis, chronic pain, stroke, allergic neuritis, progressive supranuclear palsy, Lewy body dementia (i.e., dementia with Lewy bodies or Parkinson's disease dementia), frontotemporal dementia, traumatic brain injury, traumatic spinal cord injury, multisystem atrophy, chronic traumatic encephalopathy, Creutzfeldt-Jakob disease, and leptomeningeal metastasis.

The delivery of an inhibitory RNA described herein (e.g., siRNA) to a cell can be achieved in a number of different ways. In vivo delivery may be performed by administering a composition comprising an inhibitory RNA to a subject, e.g., by parenteral administration route, e.g., subcutaneous or intravenous or intramuscular administration.

In some embodiments, an inhibitory RNA is associated with a delivery agent. “Delivery agent” refers to a substance or entity that is non-covalently or covalently associated with an inhibitory RNA or is co-administered with an inhibitory RNA and serves one or more functions that increase the stability and/or efficacy of the biologically active agent beyond that which would result if the biologically active agent was delivered (e.g., administered to a subject) in the absence of the delivery agent. For example, a delivery agent may protect an inhibitory RNA from degradation (e.g., in blood), may facilitate entry of an inhibitory RNA into cells or into a cellular compartment of interest (e.g., the cytoplasm), and/or may enhance associations with particular cells containing the molecular target to be modulated. Those of ordinary skill in the art are aware of numerous delivery agents that may be used to deliver inhibitory RNA, e.g., siRNAs. See Kanasty, R., et al. Nat Mater. 12 (11): 967-77 (2013) for review of some of these technologies. In some embodiments, e.g., for administering an inhibitory RNA systemically, the inhibitory RNA may be associated with a delivery agent such as a nanoparticle, a dendrimer, a polymer, liposomes, or a cationic delivery system. Without wishing to be bound by any theory, positively charged cationic delivery systems are believed to facilitate binding of a negatively charged inhibitory RNA and also enhance interactions at the negatively charged cell membrane to permit efficient uptake of an inhibitory RNA by the cell. Lipids (e.g., cationic lipids, or neutral lipids), dendrimers, or polymers may be bound to an inhibitory RNA or may form a vesicle or micelle that encapsulates an inhibitory RNA. Methods for making and administering complexes comprising a cationic agent and an inhibitory RNA are known in the art. In some embodiments, it is particularly contemplated to use any of the delivery agents described in US Pub. 2016/0298124. In some embodiments, an inhibitory RNA forms a complex with cyclodextrin for systemic administration. In some embodiments an inhibitory RNA is administered in association with a lipid or lipid-containing particle. In some embodiments an inhibitory RNA is administered in association with a cationic polymer (which may be a polypeptide or a non-polypeptide polymer), a lipid, a peptide, PEG, cyclodextrin, or combination thereof, which may be in the form of a nanoparticle or microparticle. The lipid or peptide may be cationic.

“Nanoparticle” refers to particles with lengths in two or three dimensions greater than 1 nanometer (nm) and smaller than about 150 nm e.g., 20 nm-50 nm or 50 nm-100 nm. “Microparticle” refers to particles with lengths in two or three dimensions greater than 150 nm and smaller than about 1000 nm. A nanoparticle may have a targeting moiety and/or cell-penetrating moiety or membrane active moiety covalently or noncovalently attached thereto. Nanoparticles, such as lipid nanoparticles, are described in, e.g., Tatiparti et al., Nanomaterials 7:77 (2017). Exemplary delivery agents, methods of manufacture and use in the delivery of inhibitory RNAs are described in U.S. Pat. Nos. 7,427,605; 8,158,601; 9,012,498; 9,415,109; 9,062,021; 9,402,816. In some embodiments, it is contemplated to use delivery technology known in the art as “Smarticles”. In some embodiments, it is contemplated to use delivery technology known in the art as “stable nucleic acid lipid particles” (SNALPs), wherein the nucleic acid to be delivered is encapsulated in a lipid bilayer containing a mixture of cationic and fusogenic lipids coated with also coated with a diffusible polyethylene glycol-lipid (PEG-lipid) conjugate that provides a neutral, hydrophilic exterior.

In some embodiments, a delivery agent comprises one or more amino alcohol cationic lipids, such as those described in U.S. Pat. No. 9,044,512.

In some embodiments, a delivery agent comprises one or more amino acid lipids. Amino acid lipids are molecules containing an amino acid residue (e.g., arginine, homoarginine, norarginine, nor-norarginine, ornithine, lysine, homolysine, histidine, 1-methylhistidine, pyridylalanine, asparagine, N-ethylasparagine, glutamine, 4-aminophenylalanine, the N-methylated versions thereof, and side chain modified derivatives thereof) and one or more lipophilic tails. Exemplary amino acid lipids and their use to deliver nucleic acids are described in US Pat. App. Pub. No. 2011/0117125 and U.S. Pat. Nos. 8,877,729, 9,139,554, and 9,339,461. In some embodiments, membrane lytic poly(amido amine) polymers and polyconjugates such as those described in U.S. Pat. App. Pub. No. 20130289207 may be used. In some embodiments, a delivery agent comprises a lipopeptide compound comprising a central peptide and having lipophilic groups attached at each terminus. In some embodiments lipophilic groups can be derived from a naturally occurring lipid. In some embodiments a lipophilic group may comprise a C(1-22)alkyl, C(6-12)cycloalkyl, C(6-12)cycloalkyl-alkyl, C(3-18)alkenyl, C(3-18)alkynyl, C(1-5)alkoxy-C(1-5)alkyl, or a sphinganine, or (2R,3R)-2-amino-1,3-octadecanediol, icosasphinganine, sphingosine, phytosphingosine, or cis-4-sphingenine. The central peptide may comprise a cationic or amphipathic amino acid sequence. Examples of such lipopeptides and their use to deliver nucleic acids are described in, e.g., U.S. Pat. No. 9,220,785.

“Masking moiety” means a molecule or group that, when physically associated with another agent (e.g., a polymer), shields, inhibits or inactivates one or more properties (biophysical or biochemical characteristics) or activities of the agent. In some embodiments, a masking moiety may be attached covalently or noncovalently to an inhibitory RNA. A masking moiety may be reversible, meaning that it is attached to the inhibitory RNA that it masks via a reversible linkage. As will be appreciated by those of ordinary skill in the art, a sufficient number of masking moieties are linked to the inhibitory RNA to be masked to achieve a desired level of inactivation.

In some embodiments an inhibitory RNA is conjugated to a delivery agent that is a polymer. Useful delivery polymers include, e.g., poly(acrylate) polymers (see., e.g., US Pat. Pub. No. 20150104408), poly(vinyl ester) polymers (see., e.g., US Pat. Pub. No. 20150110732) and certain polypeptides. In some embodiments the delivery polymer is a reversibly masked membrane active polymer. In some embodiments, the inhibitory RNA or polymer, or both, has a targeting moiety conjugated thereto. In some embodiments, an inhibitory RNA or an inhibitory RNA-targeting moiety conjugate is co-administered with a delivery polymer but is not conjugated to the polymer. “Co-administered” in this context means that the inhibitory RNA and the delivery polymer are administered to the subject such that they are present in the subject during overlapping time periods. The inhibitory RNA-targeting moiety conjugate and the delivery polymer may be administered simultaneously or they may be delivered sequentially. For simultaneous administration, they may be mixed prior to administration. For sequential administration, either the inhibitory RNA or the delivery polymer may be administered first. The inhibitory RNA and the delivery polymer may be administered in the same composition or may be administered separately sufficiently close together in time such that cytoplasmic delivery of the inhibitory RNA to cells is enhanced relative to cytoplasmic delivery that would occur without administration of the polymer. In some embodiments, the inhibitory RNA and the delivery polymer are administered no more than 15 minutes, 30 minutes, 60 minutes, or 120 minutes apart. In some embodiments, the delivery polymer is a targeted, reversibly masked membrane active polymer. The polymer has a targeting moiety attached thereto that targets the polymer to cells to which enhanced cytoplasmic delivery of the inhibitory RNA is desired. The inhibitory RNA may be targeted to the same cells, optionally using the same targeting moiety, i.e., the inhibitory RNA may be administered as an inhibitory RNA-targeting moiety conjugate. As used herein, membrane active polymers are surface active, amphipathic polymers that are able to induce one or more of the following effects upon a biological membrane: an alteration or disruption of the membrane that allows non-membrane permeable molecules to enter a cell or cross the membrane, pore formation in the membrane, fission of membranes, or disruption or dissolving of the membrane. As used herein, a membrane, or cell membrane, comprises a lipid bilayer. The alteration or disruption of the membrane can be functionally defined by the polymer's activity in at least one the following assays: red blood cell lysis (hemolysis), liposome leakage, liposome fusion, cell fusion, cell lysis, and endosomal release. A membrane active polymer may enhance delivery of a polynucleotide to a cell by disrupting or destabilizing the plasma membrane or an internal vesicle membrane (such as an endosome or lysosome), e.g., by forming a pore in the membrane, or disrupting endosomal or lysosomal vesicles thereby permitting release of the contents of the vesicle into the cell cytoplasm. In some embodiments, the targeted reversibly masked membrane active polymer is an endosomolytic polymer. Endosomolytic polymers are polymers that, in response to a change in pH, are able to cause disruption or lysis of an endosome or otherwise provide for release of a normally cell membrane impermeable compound, such as a polynucleotide or protein, from a cellular internal membrane-enclosed vesicle, such as an endosome or lysosome. In some embodiments, the polymer is a reversibly modified amphipathic membrane active polyamine wherein reversible modification inhibits membrane activity, neutralizes the polyamine to reduce positive charge and form a near neutral charge polymer. The reversible modification may also provide cell-type specific targeting and/or inhibit non-specific interactions of the polymer. The polyamine may be reversibly modified through reversible modification of amines on the polyamine. The reversibly masked membrane active polymer is substantially not membrane active when masked but becomes membrane active upon unmasking. Masking moieties are generally covalently bound to the membrane active polymer through physiologically reversible linkages. By using physiologically reversible linkages, the masking moieties can be cleaved from the polymer in vivo, thereby unmasking the polymer and restoring activity of the unmasked polymer. By choosing an appropriate reversible linkage, the activity of the membrane active polymer is restored after the conjugate has been delivered or targeted to a desired cell type or cellular location. Reversibility of the linkages provides for selective activation of the membrane active polymer. The physiologically reversible bond is reversible under mammalian intracellular conditions, which include chemical conditions such as pH, temperature, oxidative or reductive conditions or agents, and salt concentration found in or analogous to those found in mammalian cells. In some embodiments a targeting moiety, e.g., an ASGPR targeting moiety may serve as a masking moiety. In some embodiments the ASGPR targeting moiety has a lipophilic moiety conjugated thereto. Exemplary targeting moieties (e.g., ASGPR targeting moieties), physiologically labile bonds (e.g., enzymatically labile bonds, pH labile bonds), masking moieties, membrane active polymers (e.g., endosmolytically active polymers), lipophilic moieties, RNAi agent-targeting moiety conjugates, delivery agent-targeting moiety conjugates, conjugates comprising an RNAi agent, targeting moiety, and delivery agent, and methods of delivering nucleic acids to cells (e.g., liver cells) are described in US Pat. App. Pub. Nos. 2013/0245091, 2013/0317079, 2012/0157509, 2012/0165393, 2012/0172412, 2012/0230938, 2014/0135380, 2014/0135381, 2015/0104408, and 2015/0110732. In some embodiments an inhibitory RNA is co-administered with a mellitin peptide, e.g., as described in US Pat. App. Pub. No. 2012/0165393. The inhibitory RNA, mellitin peptide, or both, may have a targeting moiety conjugated thereto, optionally via a reversible linkage. In some embodiments, a masking moiety comprises a dipeptide-amidobenzyl-carbonate or disubstituted maleic anhydride masking moiety e.g., as described in US Pat. App. Pub. No. 2015/0110732.

In some embodiments, an inhibitory RNA may be administered in “naked” form, i.e., administered in the absence of a delivery agent. The naked inhibitory RNA may be in a suitable buffer solution. The buffer solution may, for example, comprise acetate, citrate, prolamine, carbonate, or phosphate, or any combination thereof. In some embodiments the buffer solution is phosphate buffered saline (PBS). The pH and osmolarity of the buffer solution can be adjusted such that it is suitable for administering to a subject. In some embodiments, an inhibitory RNA is administered not in physical association with a lipid or lipid-containing particle. In some embodiments, an inhibitory RNA is administered not in physical association with a nanoparticle or microparticle. In some embodiments, an inhibitory RNA is administered not in physical association with a cationic polymer. In some embodiments, an inhibitory RNA is administered not in physical association with cyclodextrin. In some embodiments an inhibitory RNA administered in “naked” form comprises a targeting moiety.

Inhibitory RNAs (e.g., an siRNA or miRNA described herein), or a vector comprising a nucleotide sequence encoding an siRNA or miRNA described herein, can be incorporated into pharmaceutical compositions. Such pharmaceutical compositions are useful for, among other things, administration and delivery to a subject in vivo or ex vivo. In some embodiments, pharmaceutical compositions also contain a pharmaceutically acceptable carrier or excipient. Such excipients include any pharmaceutical agent, e.g., a pharmaceutical agent that does not itself induce an immune response harmful to the individual receiving the composition, and which may be administered without undue toxicity. As used herein the terms “pharmaceutically acceptable” and “physiologically acceptable” mean a biologically acceptable formulation, gaseous, liquid or solid, or mixture thereof, which is suitable for one or more routes of administration, in vivo delivery or contact. Pharmaceutically acceptable excipients include, but are not limited to, liquids such as water, saline, glycerol, sugars and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles.

Pharmaceutical compositions may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding, free base forms. In some embodiments, a pharmaceutical composition may be a lyophilized powder.

Pharmaceutical compositions can include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules and crystals. Supplementary active compounds (e.g., preservatives, antibacterial, antiviral and antifungal agents) can also be incorporated into the compositions.

Pharmaceutical compositions can be formulated to be compatible with a particular route of administration or delivery, as set forth herein or known to one of skill in the art. Thus, pharmaceutical compositions include carriers, diluents, or excipients suitable for administration by various routes.

Compositions suitable for parenteral administration can comprise aqueous and non-aqueous solutions, suspensions or emulsions of the active compound, which preparations are typically sterile and can be isotonic with the blood of the intended recipient. Non-limiting illustrative examples include water, buffered saline, Hanks' solution, Ringer's solution, dextrose, fructose, ethanol, animal, vegetable or synthetic oils. Aqueous injection suspensions may contain substances which increase the viscosity of the suspension, such as sodium carboxymethyl cellulose, sorbitol, or dextran. Additionally, suspensions of the active compounds may be prepared as appropriate oil injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility to allow for the preparation of highly concentrated solutions.

Cosolvents and adjuvants may be added to the formulation. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Adjuvants include, for example, surfactants such as, soya lecithin and oleic acid; sorbitan esters such as sorbitan trioleate; and polyvinylpyrrolidone.

After pharmaceutical compositions have been prepared, they may be placed in an appropriate container and labeled for treatment. Such labeling can include amount, frequency, and method of administration.

Pharmaceutical compositions and delivery systems appropriate for the compositions, methods and uses of the disclosure are known in the art (see, e.g., Remington: The Science and Practice of Pharmacy. 21st Edition. Philadelphia, PA. Lippincott Williams & Wilkins, 2005).

The disclosure also provides methods for introducing inhibitory RNAs (e.g., an siRNA or miRNA described herein), or a vector comprising a nucleotide sequence encoding an siRNA or miRNA described herein, into a cell or an animal. In some embodiments, such methods include contacting a subject (e.g., a cell or tissue of a subject) with, or administering to a subject (e.g., a subject such as a mammal), an inhibitory RNA described herein (or a vector comprising a nucleotide sequence encoding an inhibitory RNA described herein), such that the inhibitory RNA is expressed in the subject (e.g., in a cell or tissue of a subject). In another embodiment, a method includes providing cells of an individual (patient or subject such as a mammal) with an inhibitory RNA described herein (or a vector comprising a nucleotide sequence encoding an inhibitory RNA described herein), such that the inhibitory RNA is expressed in the individual.

Compositions of an inhibitory RNA described herein (or a vector (e.g., an rAAV vector) comprising a nucleotide sequence encoding a inhibitory RNA described herein) can be administered in a sufficient or effective amount to a subject in need thereof. Doses can vary and depend upon the type, onset, progression, severity, frequency, duration, or probability of the disease to which treatment is directed, the clinical endpoint desired, previous or simultaneous treatments, the general health, age, gender, race or immunological competency of the subject and other factors that will be appreciated by the skilled artisan. The dose amount, number, frequency or duration may be proportionally increased or reduced, as indicated by any adverse side effects, complications or other risk factors of the treatment or therapy and the status of the subject. The skilled artisan will appreciate the factors that may influence the dosage and timing required to provide an amount sufficient for providing a therapeutic or prophylactic benefit.

The dose to achieve a therapeutic effect, e.g., the dose in vector genomes/per kilogram of body weight (vg/kg) (e.g., in the case of vector-based delivery) or mg/kg of bodyweight (mg/kg), will vary based on several factors including, but not limited to: route of administration, the level of inhibitory RNA expression required to achieve a therapeutic effect, the specific disease treated, any host immune response to the viral vector, a host immune response to the heterologous inhibitory RNA, and the stability of the inhibitory RNA expressed. One skilled in the art can determine, for vector-based deliveries of the inhibitor RNAs, a rAAV/vector genome dose range to treat a patient having a particular disease or disorder based on the aforementioned factors, as well as other factors. Generally, doses will range from at least 1×10⁸, or more, for example, 1×10⁹, 1×10¹⁰, 1×10¹¹, 1×10¹², 1×10¹³, 1×10¹⁴, or more, vector genomes per kilogram (vg/kg) of the weight of the subject, to achieve a therapeutic effect.

In some embodiments, compositions of an inhibitory RNA are administered to a subject in an amount that is between 0.01 mg/kg and 50 mg/kg. In some embodiments the inhibitory RNA composition is administered at a dose of about 0.01 mg/kg to about 10 mg/kg or about 0.5 mg/kg to about 15 mg/kg. In some embodiments, the inhibitory RNA composition is administered at a dose of about 10 mg/kg to about 30 mg/kg. In some embodiments, the inhibitory RNA composition is administered at a dose of about 0.5 mg/kg, about 1 mg/kg, about 1.5 mg/kg, about 2.0 mg/kg, about 2.5 mg/kg, about 3 mg/kg, about 3.5 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, or about 50 mg/kg. In some embodiments, the amount is between 0.01 mg/kg and 0.1 mg/kg, between 0.01 mg/kg and 0.1 mg/kg, between 0.1 mg/kg and 1.0 mg/kg, between 1.0 mg/kg and 2.5 mg/kg, between 2.5 mg/kg and 5.0 mg/kg, between 5.0 mg/kg and 10 mg/kg, between 10 mg/kg and 20 mg/kg, between 20 mg/kg and 30 mg/kg, between 30 mg/kg and 40 mg/kg or between 40 mg/kg and 50 mg/kg. In some embodiments, a fixed dose is administered. In some embodiments, the dose is between 5 mg and 1.0 g, e.g., between 5 mg and 10 mg, between 10 mg and 20 mg, between 20 mg and 40 mg, between 40 mg and 80 mg, between 80 mg and 160 mg, between 160 mg and 320 mg, between 320 mg and 640 mg, between 640 mg and 1 g. In some embodiments, the dose is about 1 mg, 5 mg, 10 mg, 25 mg, 50 mg, 100 mg, 150 mg, 200 mg, 250 mg, 300 mg, 350 mg, 400 mg, 450 mg, 500 mg, 600 mg, 700 mg, 800 mg, 900 mg, or 1000 mg. In some embodiments the dose is a daily dose. In some embodiments, the dose is administered according to a dosing regimen with a dosing interval of at least 2 days, e.g., at least 7 days, e.g., about 2, 3, 4, 6, or 8 weeks. For example, in some embodiments, the inhibitory RNA composition is administered according to a dosing regimen with a dosing interval of at least 7 days. In some embodiments, the inhibitory RNA composition is administered daily, weekly, monthly, or every 2, 3, 4, 5, or 6 months or longer. In some embodiments, any of the doses and/or dosing regimens described herein are administered subcutaneously. In some embodiments, the inhibitory RNA composition is administered once and levels of inhibition are subsequently measured, and once the level of inhibition decreases to a certain level, a subsequent dose of the inhibitory composition is administered.

In some embodiments, a subject exhibits a sustained inhibition of factor B, e.g., measured by factor B mRNA expression (e.g., in liver tissue, e.g., a liver biopsy) for a period of time that is at least 2 days, e.g., at least 7 days, e.g., about 2, 3, 4, 6, 8, 10, 12, 16, or 20 weeks post-administration. In some embodiments, a subject exhibits a reduce level of serum factor B, and the reduced level of serum factor B is maintained for a period of time that is at least 2 days, e.g., at least 7 days, e.g., about 2, 3, 4, 6, 8, 10, 12, 16, or 20 weeks post-administration.

An effective amount or a sufficient amount can (but need not) be provided in a single administration, may require multiple administrations, and, can (but need not) be, administered alone or in combination with another composition (e.g., another complement inhibitor described herein). For example, the amount may be proportionally increased as indicated by the need of the subject, type, status and severity of the disease treated or side effects (if any) of treatment. Amounts considered effective also include amounts that result in a reduction of the use of another treatment, therapeutic regimen or protocol, such as administration of another complement inhibitor described herein.

Accordingly, pharmaceutical compositions of the disclosure include compositions wherein the active ingredients are contained in an effective amount to achieve the intended therapeutic purpose. Determining a therapeutically effective dose is well within the capability of a skilled medical practitioner using the techniques and guidance provided in the disclosure. Therapeutic doses can depend on, among other factors, the age and general condition of the subject, the severity of the complement-mediated disease or disorder, and the strength of the control sequences regulating the expression levels of an inhibitory RNA described herein. Thus, a therapeutically effective amount in humans will fall in a relatively broad range that may be determined by a medical practitioner based on the response of an individual patient to vector-based treatment. Pharmaceutical compositions may be delivered to a subject, so as to allow production of an inhibitory RNA described herein in vivo by gene- and or cell-based therapies or by ex-vivo modification of the patient's or donor's cells.

Methods and uses of the disclosure include delivery and administration systemically, regionally or locally, or by any route, for example, by injection or infusion. Delivery of a pharmaceutical composition in vivo may generally be accomplished via injection using a conventional syringe, although other delivery methods such as convection-enhanced delivery can also be used (see, e.g., U.S. Pat. No. 5,720,720). For example, compositions may be delivered subcutaneously, epidermally, intradermally, intrathecally, intraorbitally, intramucosally, intraperitoneally, intravenously, intra-pleurally, intraarterially, orally, intrahepatically, intracerebroventricularly (e.g., via intracerebroventricular injection), via the portal vein, or intramuscularly. Other modes of administration include oral and pulmonary administration, suppositories, and transdermal applications. A clinician specializing in the treatment of patients with complement-mediated disorders may determine the optimal route for administration of inhibitory RNAs (e.g., an siRNA or miRNA described herein), or a vector comprising a nucleotide sequence encoding an siRNA or miRNA described herein.

In some embodiments, an inhibitory RNA described herein (or a vector comprising a nucleotide sequence encoding an inhibitory RNA described herein) may be administered to a subject once daily, weekly, every 2, 3, or 4 weeks, or even at longer intervals. In some embodiments, an inhibitory RNA described herein (or a vector comprising a nucleotide sequence encoding inhibitory RNA described herein) may be administered according to a dosing regimen that includes (i) an initial administration that is once daily, weekly, every 2, 3, or 4 weeks, or even at longer intervals; followed by (ii) a period of no administration of, e.g., 1, 2, 3, 4, 5, 6, 8, or 10 months, or 1, 2, 3, 4, 5, 6, 7, 8, 9, or 10 years. In some embodiments a vector comprising a nucleotide sequence encoding an inhibitory RNA described herein may be administered (i) one or more times during an initial time period of up to 2, 4, or 6 weeks or less; followed by (ii) a period of no administration of, e.g., 1, 2, 3, 4, 5, 6, 7, 8, 9, or 10 years. In some embodiments, a subject is monitored before and/or following treatment for level of factor B expression and/or activity, e.g., as measured using an alternative pathway assay, a classical pathway assay, or both. Suitable assays are known in the art and include, e.g., a hemolysis assay. In some embodiments, a subject is treated, or is retreated, if a measured level of factor B expression and/or activity is more than 10%, 20%, 30%, 40%, 50%, 100%, 200%, or more, relative to measured level of factor B expression and/or activity in a control subject.

### IX. Diseases, Disorders, and Conditions

Provided technologies are useful for preventing or treating various conditions, disorders or diseases, e.g., a condition, disorder or disease described herein. In some embodiments, the present disclosure provides methods for preventing a condition, disorder or disease, comprising administering to a subject susceptible thereto an effective amount of a an inhibitory RNA described herein (or a vector comprising a nucleotide sequence encoding an inhibitory RNA described herein). In some embodiments, the present disclosure provides methods for treating a condition, disorder or disease, comprising administering to a subject suffering therefrom a therapeutically effective amount of an inhibitory RNA described herein (or a vector comprising a nucleotide sequence encoding an inhibitory RNA described herein). In some embodiments, the present disclosure provides a method for reducing C3 convertase activity, comprising contacting a C3 convertase with a provided inhibitory RNA described herein (or a vector comprising a nucleotide sequence encoding an inhibitory RNA described herein). In some embodiments, the present disclosure provides a method for reducing factor B activity, comprising contacting a factor B with an inhibitory RNA described herein (or a vector comprising a nucleotide sequence encoding an inhibitory RNA described herein). In some embodiments, the present disclosure provides a method for reducing complement activation in a system, comprising administering to the system an inhibitory RNA described herein (or a vector comprising a nucleotide sequence encoding an inhibitory RNA described herein). In some embodiments, the present disclosure provides a method for reducing C3 convertase activity in a system, comprising administering to the system an inhibitory RNA described herein (or a vector comprising a nucleotide sequence encoding an inhibitory RNA described herein). In some embodiments, the present disclosure provides a method for reducing factor B activity in a system, comprising administering to the system an inhibitory RNA described herein (or a vector comprising a nucleotide sequence encoding an inhibitory RNA described herein). In some embodiments, a system is a plurality of cells, a tissue, organ or organism. In some embodiments, a system is or comprises blood. In some embodiments, a system is an animal. In some embodiments, a system is a human. In some embodiments, a subject is a human.

In some embodiments, a condition, disorder or disease is a complement-mediated condition, disorder or disease. In some embodiments, a condition, disorder or disease is a C3 convertase-mediated condition, disorder or disease. In some embodiments, a condition, disorder or disease is a factor B-mediated condition, disorder or disease. In some embodiments, a condition, disorder or disease is or comprises complement-mediated damage to an organ, tissue, or cells. In some embodiments, a compound or composition is administered in combination with another therapeutic agent, e.g., a complement inhibitor.

### A. Blood-Related Disorders

In some embodiments, an inhibitory RNA described herein (or a vector comprising a nucleotide sequence encoding an inhibitory RNA described herein), alone or in combination with one or more additional complement inhibitors described herein, is administered to a subject suffering from, or at risk of, a complement-mediated blood-related disorder, such as paroxysmal nocturnal hemoglobinuria (PNH), atypical hemolytic uremic syndrome (aHUS), autoimmune hemolytic anemia, chronic cold agglutinin disease, HELLP syndrome, and/or warm autoimmune hemolytic anemia. In some embodiments, an inhibitory RNA described herein (or a vector comprising a nucleotide sequence encoding an inhibitory RNA described herein) is administered to a subject suffering from, or at risk of, a complement-mediated disorder that affects the circulatory system. For example, in some embodiments, the disorder is thrombotic microangiopathy (TMA) or a vasculitis (e.g., IgA vasculitis) or other disorder associated with vessel inflammation, e.g., blood vessel and/or lymph vessel inflammation. In some embodiments, a vasculitis is polyarteritis nodosa, hypocomplementemic urticarial vasculitis, pulmonary vasculitis, Wegener's granulomatosis, giant cell arteritis, Churg-Strauss syndrome, microscopic polyangiitis, pauci-immune vasculitis, Henoch-Schonlein purpura, Takayasu's arteritis, Kawasaki disease, or Behcet's disease. In some embodiments, a disorder is TMA secondary to atypical hemolytic uremic syndrome. In some embodiments, a subject is positive for antineutrophil cytoplasmic antibody (ANCA).

### B. Eye Disorders

In some embodiments, an inhibitory RNA described herein (or a vector comprising a nucleotide sequence encoding an inhibitory RNA described herein) is administered to a subject for treatment of a complement-mediated eye disorder, such as macular degeneration (e.g., age-related macular degeneration (AMD) and Stargardt macular dystrophy), diabetic retinopathy, glaucoma, or uveitis (e.g., posterior uveitis or anterior uveitis). In some embodiments, a subject suffers from or is at risk of AMD. In some embodiments the AMD is neovascular (wet) AMD. In some embodiments the AMD is dry AMD. As will be appreciated by those of ordinary skill in the art, dry AMD encompasses geographic atrophy (GA), intermediate AMD, and early AMD. In some embodiments, a subject with GA is treated in order to slow or halt progression of the disease. For example, in some embodiments, treatment of a subject with GA reduces the rate of retinal cell death. A reduction in the rate of retinal cell death may be evidenced by a reduction in the rate of GA lesion growth in patients treated with an inhibitory RNA described herein (or a vector comprising a nucleotide sequence encoding an inhibitory RNA described herein), as compared with control (e.g., patients given a sham administration). In some embodiments, a subject has intermediate AMD. In some embodiments, a subject has early AMD. In some embodiments, a subject with intermediate or early AMD is treated in order to slow or halt progression of the disease. For example, in some embodiments, treatment of a subject with intermediate AMD may slow or prevent progression to an advanced form of AMD (neovascular AMD or GA). In some embodiments, treatment of a subject with early AMD may slow or prevent progression to intermediate AMD. In some embodiments an eye has both GA and neovascular AMD. In some embodiments an eye has GA but not wet AMD.

In some embodiments, a subject has an eye disorder characterized by macular degeneration, choroidal neovascularization (CNV), retinal neovascularization (RNV), ocular inflammation, or any combination of the foregoing. Macular degeneration, CNV, RNV, and/or ocular inflammation may be a defining and/or diagnostic feature of the disorder. Exemplary disorders that are characterized by one or more of these features include, but are not limited to, macular degeneration related conditions, diabetic retinopathy, retinopathy of prematurity, proliferative vitreoretinopathy, uveitis, keratitis, conjunctivitis, and scleritis. In some embodiments, a subject is in need of treatment for ocular inflammation. Ocular inflammation can affect a large number of eye structures such as the conjunctiva (conjunctivitis), cornea (keratitis), episclera, sclera (scleritis), uveal tract, retina, vasculature, and/or optic nerve. Evidence of ocular inflammation can include the presence of inflammation-associated cells such as white blood cells (e.g., neutrophils, macrophages) in the eye, the presence of endogenous inflammatory mediator(s), one or more symptoms such as eye pain, redness, light sensitivity, blurred vision and floaters, etc. Uveitis is a general term that refers to inflammation in the uvea of the eye, e.g., in any of the structures of the uvea, including the iris, ciliary body or choroid. Specific types of uveitis include iritis, iridocyclitis, cyclitis, pars planitis and choroiditis. In some embodiments, the eye disorder is Behcet's disease. In some embodiments, the eye disorder is an eye disorder characterized by optic nerve damage (e.g., optic nerve degeneration), such as glaucoma. Additional eye disorders include, e.g., retinitis pigmentosa, macular edema, Vogt-Koyangi-Harada syndrome, birdshot retino-chorioditis, sympathetic ophthalmia, ocular dicatricial pemphigoid, ocular pemphigus, nonartertic ischemic optic neuropathy, post-operative inflammation, and retinal vein occlusion.

### C. Nervous System Disorders

In some embodiments, an inhibitory RNA described herein (or a vector comprising a nucleotide sequence encoding an inhibitory RNA described herein) is used to treat a subject suffering from or at risk of a complement-mediated disorder that affects the nervous system, e.g., the central nervous system (CNS) and/or peripheral nervous system (PNS). Examples of such disorders include, e.g., a neurodegenerative disorder such as multiple sclerosis, other demyelinating diseases (e.g., neuromyelits optica or chronic inflammatory demyelinating polyneuropathy (CIDP)), amyotrophic lateral sclerosis, chronic pain, fibromyalgia, stroke, intracerebral hemorrhage, allergic neuritis, diabetic neuropathy, Huntington's disease, schizophrenia, Alzheimer's disease, Parkinson's disease, progressive supranuclear palsy, Lewy body dementia (i.e., dementia with Lewy bodies or Parkinson's disease dementia), frontotemporal dementia, progressive supranuclear palsy, corticobasal syndrome, Pick's disease, mild cognitive impairment, traumatic brain injury, traumatic spinal cord injury, multisystem atrophy, chronic traumatic encephalopathy, Creutzfeldt-Jakob disease, Guillain Barre Syndrome, and leptomeningeal metastasis. In some embodiments, a subject suffers from neuropathic pain, e.g., arising from lesions that involve the somatosensory pathways with damage to small fibres in peripheral nerves and/or to the spino-thalamocortical system in the CNS.

### D. Kidney Disorders

In some embodiments, an inhibitory RNA described herein (or a vector comprising a nucleotide sequence encoding an inhibitory RNA described herein) is used to treat a subject suffering from, or at risk of, a complement-mediated kidney disorder. Such disorders include, e.g., nephritis, e.g., glomerulonephritis, e.g., membranoproliferative glomerulonephritis (MPGN) (e.g., MPGN type I, MPGN type II, or MPGN type III), e.g., immune complex membranoproliferative glomerulonephritis (IC-MPGN). In some embodiments the disorder is IgA nephropathy (IgAN), primary membranous nephropathy, or diabetic nephropathy. In some embodiments, the disorder is polycystic kidney disease (PKD). In some embodiments, the disorder is C3 glomerulopathy. In some embodiments the disorder is characterized by glomerular deposits containing one or more complement activation products, e.g., C3b, in the kidney. In some embodiments treatment as described herein reduces the level of such deposits. In some embodiments a subject suffering from a complement-mediated kidney disorder suffers from proteinuria (an abnormally high level of protein in the urine) and/or an abnormally low glomerular filtration rate (GFR). In some embodiments treatment as described herein results in decreased proteinuria and/or an increased or stabilized GFR.

### E. Respiratory Disorders

In some embodiments, an inhibitory RNA described herein (or a vector comprising a nucleotide sequence encoding an inhibitory RNA described herein) is used to treat a subject suffering from or at risk of a complement-mediated disorder respiratory disorder. In some embodiments, a subject is suffering from or at risk of acute respiratory distress syndrome. In some embodiments, a respiratory disease is, e.g., asthma (e.g., allergic asthma), emphysema, chronic inflammation, chronic obstructive pulmonary disease (COPD), pulmonary fibrosis (e.g., idiopathic pulmonary fibrosis), radiation-induced lung injury, allergic bronchopulmonary aspergillosis, hypersensitivity pneumonitis (also known as allergic alveolitis), eosinophilic pneumonia, interstitial pneumonia, sarcoid, Wegener's granulomatosis, pulmonary embolisms and infarcts, dyspnea, hemoptysis, bronchoconstriction, or bronchiolitis obliterans.

### F. Musculoskeletal Disorders

In some embodiments, an inhibitory RNA described herein (or a vector comprising a nucleotide sequence encoding an inhibitory RNA described herein) is used to treat a subject suffering from, or at risk of, a complement-mediated disorder that affects the musculoskeletal system. Examples of such disorders include inflammatory joint conditions (e.g., arthritis such as rheumatoid arthritis or psoriatic arthritis, juvenile chronic arthritis, spondyloarthropathies Reiter's syndrome, gout). In some embodiments, a musculoskeletal system disorder results in symptoms such as pain, stiffness and/or limitation of motion of the affected body part(s). Inflammatory myopathies include dermatomyositis, polymyositis, and various others are disorders of chronic muscle inflammation of unknown etiology that result in muscle weakness. In some embodiments, a complement-mediated musculoskeletal disorder is myasthenia gravis.

### G. Transplantation

In some embodiments, an inhibitory RNA described herein (or a vector comprising a nucleotide sequence encoding an inhibitory RNA described herein) is used to protect a graft from complement-mediated damage. A graft can be contacted with an inhibitory RNA described herein (or a vector comprising a nucleotide sequence encoding an inhibitory RNA described herein) prior to, during, and/or after being transplanted, in various embodiments of the disclosure. In another embodiment, an inhibitory RNA described herein (or a vector comprising a nucleotide sequence encoding an inhibitory RNA described herein) is administered to a donor prior to removal of the graft. In some embodiments, an inhibitory RNA described herein (or a vector comprising a nucleotide sequence encoding an inhibitory RNA described herein) is administered to a recipient during and/or after the introduction of the graft. In some embodiments, an inhibitory RNA described herein (or a vector comprising a nucleotide sequence encoding an inhibitory RNA described herein) is administered to a recipient prior to the introduction of the graft. In some embodiments, a subject receives an inhibitory RNA described herein (or a vector comprising a nucleotide sequence encoding an inhibitory RNA described herein) after receiving the graft.

In some embodiments, a graft is or comprises a solid organ such as a kidney, liver, lung, pancreas, or heart. In some embodiments, a graft is or comprises bone, cartilage, fascia, tendon, ligament, cornea, sclera, pericardium, skin, heart valve, blood vessel, amniotic membrane, or dura mater. In some embodiments, a graft comprises multiple organs such as a heart-lung or pancreas-kidney graft. In some embodiments, a graft comprises less than a complete organ or tissue. For example, a graft may contain a portion of an organ or tissue, e.g., a liver lobe, section of blood vessel, skin flap, or heart valve. In some embodiments, a graft comprises a preparation comprising isolated cells or tissue fragments that have been isolated from their tissue of origin but retain at least some tissue architecture, e.g., pancreatic islets. In some embodiments, a preparation comprises isolated cells that are not attached to each other via connective tissue, e.g., hematopoietic stem cells or progenitor cells derived from peripheral and/or cord blood, or whole blood or any cell-containing blood product such as red blood cells (RBCs) or platelets.

In some embodiments, a graft is a xenograft (i.e., the donor and recipient are of different species), an autograft (i.e., a graft from one part of the body to another part of the body in the same individual), an isograft (i.e., the donor and recipient are genetically identical), or an allograft (i.e., the donor and recipient are genetically non-identical members of the same species).

### H. Ischemia/Reperfusion Injury

Ischemia-reperfusion (I/R) injury is an important cause of tissue damage following trauma and in other conditions associated with temporary disruption of blood flow such as myocardial infarction, stroke, severe infection, vascular disease, aneurysm repair, cardiopulmonary bypass, and transplantation. In the setting of trauma, systemic hypoxemia, hypotension, and local interruption of the blood supply resulting from contusions, compartment syndrome, and vascular injuries cause ischemia that damages metabolically active tissues. Restoration of the blood supply triggers an intense systemic inflammatory reaction. After reperfusion, all three major complement pathways are activated and, acting cooperatively or independently, are involved in I/R related adverse events affecting numerous organ systems.

In some embodiments an inhibitory RNA described herein (or a vector comprising a nucleotide sequence encoding an inhibitory RNA described herein) is administered to a subject who has recently (e.g., within the preceding 2, 4, 8, 12, 24, or 48 hours) experienced trauma, e.g., trauma that puts the subject at risk of I/R injury, e.g., due to systemic hypoxemia, hypotension, and/or local interruption of the blood supply. In some embodiments, an inhibitory RNA described herein (or a vector comprising a nucleotide sequence encoding an inhibitory RNA described herein) may be administered intravascularly, optionally into a blood vessel that supplies an injured body part or directly to the body part. In some embodiments, the subject suffers from spinal cord injury, traumatic brain injury, burn, and/or hemorrhagic shock. In some embodiments, an inhibitory RNA described herein (or a vector comprising a nucleotide sequence encoding an inhibitory RNA described herein) is administered to a subject prior to, during, or after a surgical procedure, e.g., a surgical procedure that is expected to temporarily disrupt blood flow to a tissue, organ, or portion of the body. Examples of such procedures include cardiopulmonary bypass, angioplasty, heart valve repair/replacement, aneurysm repair, or other vascular surgeries. An an inhibitory RNA described herein (or a vector comprising a nucleotide sequence encoding an inhibitory RNA described herein) may be administered prior to, after, and/or during an overlapping time period with the surgical procedure.

In some embodiments, an inhibitory RNA described herein (or a vector comprising a nucleotide sequence encoding an inhibitory RNA described herein) is administered to a subject who has suffered an MI, thromboembolic stroke, deep vein thrombosis, or pulmonary embolism. An an inhibitory RNA described herein (or a vector comprising a nucleotide sequence encoding an inhibitory RNA described herein) may be administered in combination with a thrombolytic agent such as tissue plasminogen activator (tPA) (e.g., alteplase (Activase), reteplase (Retavase), tenecteplase (TNKase)), anistreplase (Eminase), streptokinase (Kabikinase, Streptase), or urokinase (Abbokinase). In some embodiments, an inhibitory RNA described herein (or a vector comprising a nucleotide sequence encoding an inhibitory RNA described herein) may be administered prior to, after, and/or during an overlapping time period with the thrombolytic agent.

### I. Other Disorders

In some embodiments, an inhibitory RNA described herein (or a vector comprising a nucleotide sequence encoding an inhibitory RNA described herein) is used to treat a subject suffering from, or at risk of, a complement-mediated disorder that affects the integumentary system. Examples of such disorders include, e.g., atopic dermatitis, psoriasis, pemphigoid, pemphigus, systemic lupus erythematosus, dermatomyositis, scleroderma, sclerodermatomyositis, Sjögren syndrome, and chronic urticaria.

In some embodiments, an inhibitory RNA described herein (or a vector comprising a nucleotide sequence encoding an inhibitory RNA described herein) is used to treat a subject suffering from, or at risk of, a complement-mediated disorder that affects the gastrointestinal system, e.g., inflammatory bowel disease, e.g., Crohn's disease or ulcerative colitis.

In some embodiments, an inhibitory RNA described herein (or a vector comprising a nucleotide sequence encoding an inhibitory RNA described herein) is used to treat a subject suffering from, or at risk of, a complement-mediated inflammatory disorder, such as rhinosinusitis or myocarditis.

In some embodiments, an inhibitory RNA described herein (or a vector comprising a nucleotide sequence encoding an inhibitory RNA described herein) is used to treat a subject suffering from, or at risk of, thyroiditis (e.g., Hashimoto's thyroiditis, Graves' disease, post-partum thyroiditis), hepatitis (e.g., hepatitis C), pancreatitis, panniculitis, or MYH9-related disorders.

In some embodiments, an inhibitory RNA described herein (or a vector comprising a nucleotide sequence encoding an inhibitory RNA described herein) is used to treat interleukin-2 induced toxicity during IL-2 therapy, myocardial infarction, post-pump syndrome in cardiopulmonary bypass or renal bypass, atherosclerosis, hemodialysis, renal ischemia, mesenteric artery reperfusion after aortic reconstruction, infectious disease or sepsis, immune complex disorders and autoimmune diseases, liver fibrosis, fibrogenic dust diseases, nasal polyposis, parasitic diseases, Goodpasture's Syndrome, immune complex-associated inflammation, antiphospholipid syndrome, cancer, periodontitis, gingivitis, or obesity.

In some embodiments, a complement-mediated condition, disorder or disease is complement activation secondary to administration of another therapeutic or diagnostic agent. For example, in some embodiments, a complement-mediated condition, disorder or disease is complement activation secondary to gene therapy (e.g., gene therapy with a viral vector such as an adeno-associated virus (AAV), adenovirus, or lentivirus vector) or complement activation secondary to cell therapy). In some embodiments, a subject suffers from TMA secondary to hematopoietic stem cell transplant (HSCT-TMA). In some embodiments, a subject suffers from drug-induced TMA. In some embodiments, administration of an inhibitory RNA described herein (or a vector comprising a nucleotide sequence encoding an inhibitory RNA described herein) described herein prior to and/or following administration of another therapeutic agent may increase efficacy and/or safety of said therapeutic agent.

### X. Combination Therapy

In some aspects, methods of the present disclosure involve administering an inhibitory RNA described herein, alone or in combination with one or more additional therapies that modulate an immune response (e.g., one or more additional complement inhibitors). In some embodiments, an inhibitory RNA is administered to a subject already receiving therapy with another immunomodulatory therapy (e.g., another complement inhibitor); in some embodiments, another immunomodulatory therapy (e.g., another complement inhibitor) is administered to a subject receiving an inhibitory RNA. In some embodiments, both an inhibitory RNA and another immunomodulatory therapy (e.g., complement inhibitor) are administered to the subject.

Examples of immunomodulatory therapies include other complement inhibitors, including those described herein. In some embodiments, an immunomodulatory therapy is includes a cancer vaccine, an adoptive T cell or antibody therapy, an immune checkpoint blockade or a combination thereof. In some embodiments, an immunomodulatory therapy includes agents such as interleukins such (e.g., IL-2, IL-7, IL-12); cytokines such as granulocyte-macrophage colony-stimulating factor (GM-CSF), interferons; various chemokines such as CXCL13, CCL26, CXCL7; antagonists of immune checkpoint blockades such as anti-CTLA-4, anti-PD-1, anti-PD-LI, anti-LAG3 and anti-B7-H3; synthetic cytosine phosphate-guanosine (CpG), oligodeoxynucleotides, glucans, modulators of regulatory T cells (Tregs) such as cyclophosphamide, or other immune modulating agents. In one embodiment, an immunomodulatory therapy includes an agonist antibody to 4-IBB (CD 137). In some embodiments, an immunomodulatory therapy is macrophage modulator such as Bindarit. In some embodiments, an immunomodulatory therapy is a TNFα inhibitor such as Humira. In some embodiments administration of an inhibitory RNA may allow for administering a reduced dosing regimen of (e.g., involving a smaller amount in an individual dose, reduced frequency of dosing, reduced number of doses, and/or reduced overall exposure to) a second complement inhibitor, as compared to administration of a second complement inhibitor as single therapy. Without wishing to be bound by any theory, in some embodiments a reduced dosing regimen of a second complement inhibitor may avoid one or more undesired adverse effects that could otherwise result.

In some aspects, administration of an inhibitory RNA in combination with a second complement inhibitor can reduce the amount of factor B in the subject's blood sufficiently such that a reduced dosing regimen of an inhibitory RNA and/or the second complement inhibitor is required to achieve a desired degree of complement inhibition.

In some aspects, administration of an inhibitory RNA in combination with a second complement inhibitor can reduce the amount of factor B in the subject's blood sufficiently such that a reduced dosing regimen of an inhibitory RNA and/or the second complement inhibitor is required to achieve a desired level of, or a desired amount of improvement in, one or more signs, symptoms, biomarkers, or outcome measures, of a complement-mediated disorder.

In some embodiments such a reduced dose can be administered in a smaller volume, or using a lower concentration, or using a longer dosing interval, or any combination of the foregoing, as compared to administration of an inhibitory RNA or a second complement inhibitor as single therapy.

Any complement inhibitor, e.g., a complement inhibitor known in the art, can be administered in combination with an inhibitory RNA described herein. In some embodiments, a complement inhibitor is compstatin or a compstatin analog.

Compstatin is a cyclic peptide that binds to C3 and inhibits complement activation. U.S. Pat. No. 6,319,897 describes a peptide having the sequence Ile-[Cys-Val-Val-Gln-Asp-Trp-Gly-His-His-Arg-Cys]-Thr (SEQ ID NO: 1), with the disulfide bond between the two cysteines denoted by brackets. It will be understood that the name “compstatin” was not used in U.S. Pat. No. 6,319,897 but was subsequently adopted in the scientific and patent literature (see, e.g., Morikis, et al., Protein Sci., 7 (3): 619-27, 1998) to refer to a peptide having the same sequence as SEQ ID NO: 2 disclosed in U.S. Pat. No. 6,319,897, but amidated at the C terminus. The term “compstatin” is used herein consistently with such usage. Compstatin analogs that have higher complement inhibiting activity than compstatin have been developed. See, e.g., WO2004/026328 (PCT/US2003/029653), Morikis, D., et al., Biochem Soc Trans. 32 (Pt 1): 28-32, 2004, Mallik, B., et al., J. Med. Chem., 274-286, 2005; Katragadda, M., et al. J. Med. Chem., 49:4616-4622, 2006; WO2007062249 (PCT/US2006/045539); WO2007044668 (PCT/US2006/039397), WO/2009/046198 (PCT/US2008/078593); WO/2010/127336 (PCT/US2010/033345).

As used herein, the term “compstatin analog” includes compstatin and any complement inhibiting analog thereof. The term “compstatin analog” encompasses compstatin and other compounds designed or identified based on compstatin and whose complement inhibiting activity is at least 50% as great as that of compstatin as measured, e.g., using any complement activation assay accepted in the art or substantially similar or equivalent assays. Certain suitable assays are described in U.S. Pat. No. 6,319,897, WO2004/026328, Morikis, supra, Mallik, supra, Katragadda 2006, supra, WO2007062249 (PCT/US2006/045539); WO2007044668 (PCT/US2006/039397), WO/2009/046198 (PCT/US2008/078593); and/or WO/2010/127336 (PCT/US2010/033345). The assay may, for example, measure alternative or classical pathway-mediated erythrocyte lysis or be an ELISA assay. In some embodiments, an assay described in WO/2010/135717 (PCT/US2010/035871) is used.

Table 7 provides a non-limiting list of compstatin analogs useful in the present disclosure. The analogs are referred to in abbreviated form in the left column by indicating specific modifications at designated positions (1-13) as compared to the parent peptide, compstatin. Consistent with usage in the art, “compstatin” as used herein, and the activities of compstatin analogs described herein relative to that of compstatin, refer to the compstatin peptide amidated at the C-terminus. Unless otherwise indicated, peptides in Table 7 are amidated at the C-terminus. Bold text is used to indicate certain modifications. Activity relative to compstatin is based on published data and assays described therein (WO2004/026328, WO2007044668, Mallik, 2005; Katragadda, 2006). In certain embodiments, the peptides listed in Table 7 are cyclized via a disulfide bond between the two Cys residues when used in the therapeutic compositions and methods of the disclosure. Alternate means for cyclizing the peptides are also within the scope of the disclosure.

TABLE 7

| | | | Activity |
|---|---|---|---|
| | | SEQ | over |
| | | ID | comp- |
| Peptide | Sequence | NO: | statin |
| Compstatin | H-ICVVQDWGHHRCT-CONH2 | 8 | * |
| Ac-compstatin | Ac-ICVVQDWGHHRCT- | 9 | 3xmore |
| | CONH2 | | |
| Ac-V4Y/H9A | Ac-ICVYQDWGAHRCT- | 10 | 14xmore |
| | CONH2 | | |
| Ac-V4W/H9A-OH | Ac-ICVWQDWGAHRCT-COOH | 11 | 27xmore |
| Ac-V4W/H9A | Ac-ICVWQDWGAHRCT- | 12 | 45xmore |
| | CONH2 | | |
| Ac-V4W/H9A/ | Ac-ICVWQDWGAHRCdT- | 13 | 55xmore |
| T13dT-OH | COOH | | |
| Ac-V4(2-Nal)/ | Ac-ICV(2-Nal) | 14 | 99xmore |
| H9A | CQDWGAHRCT-ONH2 | | |
| Ac V4(2-Nal)/ | Ac-ICV(2-Nal) | 15 | 38xmore |
| H9A-OH | QDWGAHRCT-COOH | | |
| Ac V4(1-Nal)/ | Ac-ICV(1-Nal) | 16 | 30xmore |
| H9A-OH | QDWGAHRCT-COOH | | |
| Ac-V42Igl/H9A | Ac-ICV(2-Igl) | 17 | 39xmore |
| | QDWGAHRCT-CONH2 | | |
| Ac-V42Igl/H9A- | Ac-ICV(2-Igl) | 18 | 37xmore |
| OH | QDWGAHRCT-COOH | | |
| Ac-V4Dht/H9A- | Ac-ICVDhtQDWGAHRCT- | 19 | 5xmore |
| OH | COOH | | |
| Ac-V4(Bpa)/ | Ac-ICV(Bpa)QDWGAHRCT- | 20 | 49xmore |
| H9A-OH | COOH | | |
| Ac-V4(Bpa)/ | Ac-ICV(Bpa)QDWGAHRCT- | 21 | 86xmore |
| H9A | CONH2 | | |
| Ac-V4(Bta)/ | Ac-ICV(Bta)QDWGAHRCT- | 22 | 65xmore |
| H9A-OH | COOH | | |
| Ac-V4(Bta)/ | Ac-ICV(Bta)QDWGAHRCT- | 23 | 64xmore |
| H9A | CONH2 | | |
| Ac-V4W/H9 | Ac-ICVWQDWG(2-Abu) | 24 | 64xmore |
| (2-Abu) | HRCT-CONH2 | | |
| +G/V4W/H9A | H-GICVWQDWGAHRCTAN- | 25 | 38xmore |
| +AN -OH | COOH | | |
| Ac-V4(5fW)/ | Ac-ICV(5fW)QDWGAHRCT- | 26 | 31xmore |
| H9A | CONH₂ | | |
| Ac-V4(5-MeW)/ | Ac-ICV(5-methyl-W) | 27 | 67xmore |
| H9A | QDWGAHRCT-CONH₂ | | |
| Ac-V4(1-MeW)/ | Ac-ICV(1-methyl-W) | 28 | 264xmore |
| H9A | QDWGAHRCT-CONH₂ | | |
| Ac-V4W/W7 | Ac-ICVWQD(5fW) | 29 | 121xmore |
| (5fW)/H9A | GAHRCT-CONH₂ | | |
| Ac-V4(5fW)/ | Ac-ICV(5fW)QD(5fW) | 30 | NA |
| W7(5fW)/H9A | GAHRCT-CONH₂ | | |
| Ac-V4(5-MeW)/ | Ac-ICV(5-methyl-W)QD | 31 | NA |
| W7(5fW)H9A | C(5fW)GAHRCT-ONH₂ | | |
| Ac-V4(1MeW)/ | Ac-ICV(1-methyl-W)QD | 32 | 264xmore |
| W7(5fW)/H9A | C(5fW)GAHRCT-ONH₂ | | |
| +G/V4(6fW)/W7 | H-GICV(6fW)QD(6fW) | 33 | 126xmore |
| (6fW)H9A + | GAHRCTN-COOH | | |
| N-OH | | | |
| Ac-V4(1- | Ac-ICV(1-formyl-W) | 34 | 264xmore |
| formyl-W)/H9A | QDWGAHRCT-CONH₂ | | |
| Ac-V4(5- | Ac-ICV(1-methyoxy-W) | 35 | 76xmore |
| methoxy-W)/H9A | QDWGAHRCT-CONH₂ | | |
| G/V4(5f-W)/W7 | H-GICV(5fW)QD(5fW) | 36 | 112xmore |
| (5fW)/ | GAHRCTN-COOH | | |
| H9A + N-OH | | | |

NA = not available

In certain embodiments of the compositions and methods of the disclosure, the compstatin analog has a sequence selected from sequences 9-36. In one embodiment, the compstatin analog has a sequence of SEQ ID NO: 28. As used herein, “L-amino acid” refers to any of the naturally occurring levorotatory alpha-amino acids normally present in proteins or the alkyl esters of those alpha-amino acids. The term “D-amino acid” refers to dextrorotatory alpha-amino acids. Unless specified otherwise, all amino acids referred to herein are L-amino acids.

In some embodiments, one or more amino acid(s) of a compstatin analog (e.g., any of the compstatin analogs disclosed herein) can be an N-alkyl amino acid (e.g., an N-methyl amino acid). For example, and without limitation, at least one amino acid within the cyclic portion of the peptide, at least one amino acid N-terminal to the cyclic portion, and/or at least one amino acid C-terminal to the cyclic portion may be an N-alkyl amino acid, e.g., an N-methyl amino acid. In some embodiments, for example, a compstatin analog comprises an N-methyl glycine, e.g., at the position corresponding to position 8 of compstatin and/or at the position corresponding to position 13 of compstatin. In some embodiments, one or more of the compstatin analogs in Table 7 contains at least one N-methyl glycine, e.g., at the position corresponding to position 8 of compstatin and/or at the position corresponding to position 13 of compstatin. In some embodiments, one or more of the compstatin analogs in Table 7 contains at least one N-methyl isoleucine, e.g., at the position corresponding to position 13 of compstatin. For example, a Thr at or near the C-terminal end of a peptide whose sequence is listed in Table 7 or any other compstatin analog sequence may be replaced by N-methyl Ile. As will be appreciated, in some embodiments the N-methylated amino acids comprise N-methyl Gly at position 8 and N-methyl Ile at position 13. In some embodiments, a compstatin analog (e.g., any one of the compstatin analogs listed in Table 7) comprises an isoleucine at position corresponding to position 3 of SEQ ID NO: 8, either instead of or in addition to one or more substitutions described herein. For example, in some embodiments, a compstatin analog comprises or consists of the sequence of any one of SEQ ID NOs: 8-36, where position 3 is an isoleucine. In some embodiments, a compstatin analog comprises or consists of the sequence of any one of SEQ ID NOs: 25, 33, or 36, where position 4 is an isoleucine. Additional compstatin analogs are described in, e.g., WO2019/166411.

Compstatin analogs may be prepared by various synthetic methods of peptide synthesis known in the art via condensation of amino acid residues, e.g., in accordance with conventional peptide synthesis methods, may be prepared by expression in vitro or in living cells from appropriate nucleic acid sequences encoding them using methods known in the art. For example, peptides may be synthesized using standard solid-phase methodologies as described in Malik, supra, Katragadda, supra, WO2004026328, and/or WO2007062249. Potentially reactive moieties such as amino and carboxyl groups, reactive functional groups, etc., may be protected and subsequently deprotected using various protecting groups and methodologies known in the art. See, e.g., “Protective Groups in Organic Synthesis”, 3rd ed. Greene, T. W. and Wuts, P. G., Eds., John Wiley & Sons, New York: 1999. Peptides may be purified using standard approaches such as reversed-phase HPLC. Separation of diasteriomeric peptides, if desired, may be performed using known methods such as reversed-phase HPLC. Preparations may be lyophilized, if desired, and subsequently dissolved in a suitable solvent, e.g., water. The pH of the resulting solution may be adjusted, e.g. to physiological pH, using a base such as NaOH. Peptide preparations may be characterized by mass spectrometry if desired, e.g., to confirm mass and/or disulfide bond formation. See, e.g., Mallik, 2005, and Katragadda, 2006.

A compstatin analog can be modified by addition of a molecule such as polyethylene glycol (PEG) to stabilize the compound, reduce its immunogenicity, increase its lifetime in the body, increase or decrease its solubility, and/or increase its resistance to degradation. Methods for pegylation are well known in the art (Veronese, F. M. & Harris, Adv. Drug Deliv. Rev. 54, 453-456, 2002; Davis, F. F., Adv. Drug Deliv. Rev. 54, 457-458, 2002); Hinds, K. D. & Kim, S. W. Adv. Drug Deliv. Rev. 54, 505-530 (2002; Roberts, M. J., Bentley, M. D. & Harris, J. M. Adv. Drug Deliv. Rev. 54, 459-476; 2002); Wang, Y. S. et al. Adv. Drug Deliv. Rev. 54, 547-570, 2002). A wide variety of polymers such as PEGs and modified PEGs, including derivatized PEGs to which polypeptides can conveniently be attached are described in Nektar Advanced Pegylation 2005-2006 Product Catalog, Nektar Therapeutics, San Carlos, CA, which also provides details of appropriate conjugation procedures.

In some embodiments, a compstatin analog of any of SEQ ID NOs: 9-36, is extended by one or more amino acids at the N-terminus, C-terminus, or both, wherein at least one of the amino acids has a side chain that comprises a reactive functional group such as a primary or secondary amine, a sulfhydryl group, a carboxyl group (which may be present as a carboxylate group), a guanidino group, a phenol group, an indole ring, a thioether, or an imidazole ring, which facilitate conjugation with a reactive functional group to attach a PEG to the compstatin analog. In some embodiments, the compstatin analog comprises an amino acid having a side chain comprising a primary or secondary amine, e.g., a Lys residue. For example, a Lys residue, or a sequence comprising a Lys residue, is added at the N-terminus and/or C-terminus of a compstatin analog described herein (e.g., a compstatin analog comprising any one of SEQ ID NOs: 9-36).

In some embodiments, the Lys residue is separated from the cyclic portion of the compstatin analog by a rigid or flexible spacer. The spacer may, for example, comprise a substituted or unsubstituted, saturated or unsaturated alkyl chain, oligo (ethylene glycol) chain, and/or other moieties, e.g., as described herein with regard to linkers. The length of the chain may be, e.g., between 2 and 20 carbon atoms. In other embodiments the spacer is a peptide. The peptide spacer may be, e.g., between 1 and 20 amino acids in length, e.g., between 4 and 20 amino acids in length. Suitable spacers can comprise or consist of multiple Gly residues, Ser residues, or both, for example. Optionally, the amino acid having a side chain comprising a primary or secondary amine and/or at least one amino acid in a spacer is a D-amino acid. Any of a variety of polymeric backbones or scaffolds could be used. For example, the polymeric backbone or scaffold may be a polyamide, polysaccharide, polyanhydride, polyacrylamide, polymethacrylate, polypeptide, polyethylene oxide, or dendrimer. Suitable methods and polymeric backbones are described, e.g., in WO98/46270 (PCT/US98/07171) or WO98/47002 (PCT/US98/06963). In one embodiment, the polymeric backbone or scaffold comprises multiple reactive functional groups, such as carboxylic acids, anhydride, or succinimide groups. The polymeric backbone or scaffold is reacted with the compstatin analogs. In one embodiment, the compstatin analog comprises any of a number of different reactive functional groups, such as carboxylic acids, anhydride, or succinimide groups, which are reacted with appropriate groups on the polymeric backbone. Alternately, monomeric units that could be joined to one another to form a polymeric backbone or scaffold are first reacted with the compstatin analogs and the resulting monomers are polymerized. In another embodiment, short chains are prepolymerized, functionalized, and then a mixture of short chains of different composition are assembled into longer polymers.

In some embodiments, a compstatin analog moiety is attached at each end of a linear PEG. A bifunctional PEG having a reactive functional group at each end of the chain may be used, e.g., as described herein. In some embodiments, the reactive functional groups are identical while in some embodiments different reactive functional groups are present at each end.

In general and compounds depicted herein, a polyethylene glycol moiety is drawn with the oxygen atom on the right side of the repeating unit or the left side of the repeating unit. In cases where only one orientation is drawn, the present disclosure encompasses both orientations (i.e., (CH₂CH₂O), and (OCH₂CH₂) n) of polyethylene glycol moieties for a given compound or genus, or in cases where a compound or genus contains multiple polyethylene glycol moieties, all combinations of orientations are encompasses by the present disclosure.

In some embodiments a bifunctional linear PEG comprises a moiety comprising a reactive functional group at each of its ends. The reactive functional groups may be the same (homobifunctional) or different (heterobifunctional). In some embodiments the structure of a bifunctional PEG may be symmetric, wherein the same moiety is used to connect the reactive functional group to oxygen atoms at each end of the —(CH₂CH₂O)ₙ chain. In some embodiments different moieties are used to connect the two reactive functional groups to the PEG portion of the molecule. The structures of exemplary bifunctional PEGs are depicted below. For illustrative purposes, formulas in which the reactive functional group(s) comprise an NHS ester are depicted, but other reactive functional groups could be used.

In some embodiments, a bifunctional linear PEG is of formula A:

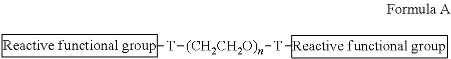

wherein each T and “Reactive functional group” is independently as defined below, and described in classes and subclasses herein, and n is as defined above and described in classes and subclasses herein.
Each T is independently a covalent bond or a C1-12 straight or branched, hydrocarbon chain wherein one or more carbon units of T are optionally and independently replaced by —O—, —S—, —N(R^{x})—, —C(O)—, —C(O)O—, —OC(O)—, —N(R^{x})C(O)—, —C(O)N(R^{x})—, —S(O)—, —S(O)₂—, —N(R^{x})SO₂—, or —SO₂N(R^{x})—; and
each R^{x} is independently hydrogen or C₁₋₆ aliphatic.
The Reactive functional group has the structure —COO—NHS.

Exemplary bifunctional PEGs of formula A include:

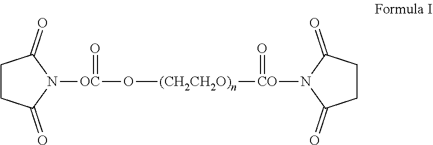

In some embodiments, a functional group (for example, an amine, hydroxyl, or thiol group) on a compstatin analog is reacted with a PEG-containing compound having a “reactive functional group” as described herein, to generate such conjugates. By way of example, Formula I can form compstatin analog conjugates having the structure:

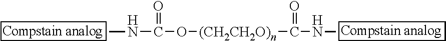

wherein,

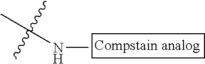

represents the attachment point of an amine group on a compstatin analog. In certain embodiments, an amine group is a lysine side chain group.

In certain embodiments, the PEG component of such conjugates has an average molecular weight of about 5 kD, about 10 kD, about 15 kD, about 20 kD, about 30 kD, or about 40 kD. In certain embodiments, the PEG component of such conjugates has an average molecular weight of about 40 kD.

The term “bifunctional” or “bifunctionalized” is sometimes used herein to refer to a compound comprising two compstatin analog moieties linked to a PEG. Such compounds may be designated with the letter “BF”. In some embodiments a bifunctionalized compound is symmetrical. In some embodiments the linkages between the PEG and each of the compstatin analog moieties of a bifunctionalized compound are the same. In some embodiments, each linkage between a PEG and a compstatin analog of a bifunctionalized compound comprises a carbamate. In some embodiments, each linkage between a PEG and a compstatin analog of a bifunctionalized compound comprises a carbamate and does not comprise an ester. In some embodiments, each compstatin analog of a bifunctionalized compound is directly linked to a PEG via a carbamate. In some embodiments, each compstatin analog of a bifunctionalized compound is directly linked to a PEG via a carbamate, and the bifunctionalized compound has the structure:

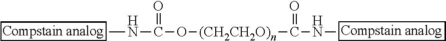

In some embodiments of formulae and embodiments described herein,

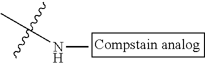

represents point of attachment of a lysine side chain group in a compstatin analog having the structure:

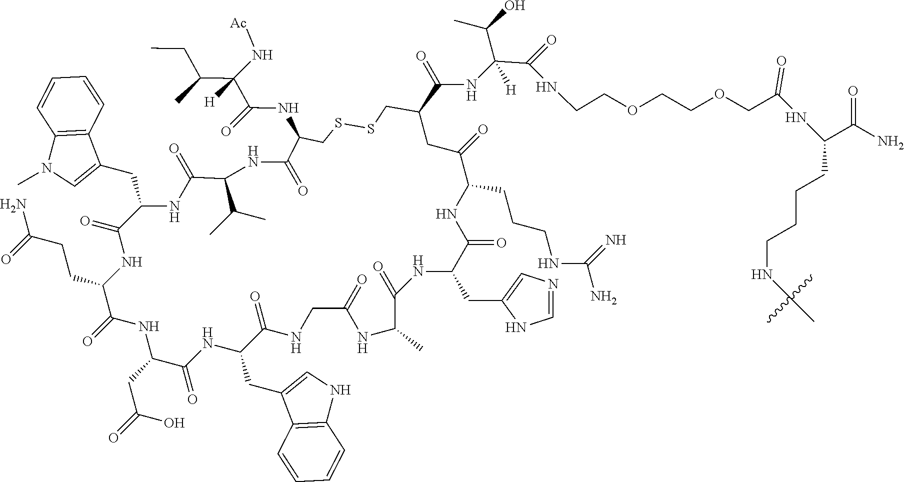 [SMILES: *NCCCC[C@H](NC(=O)COCCOCCNC(=O)[C@@H](NC(=O)[C@@H]1CSSC[C@H](NC(=O)[C@@H](NC(C)=O)[C@@H](C)CC)C(=O)N[C@@H](C(C)C)C(=O)N[C@@H](Cc2cn(C)c3ccccc23)C(=O)N[C@@H](CCC(N)=O)C(=O)N[C@@H](CC(=O)O)C(=O)N[C@@H](Cc2c[nH]c3ccccc23)C(=O)NCC(=O)N[C@@H](C)C(=O)N[C@@H](Cc2cnc[nH]2)C(=O)N[C@@H](CCCNC(=N)N)C(=O)C1)[C@@H](C)O)C(N)=O]

wherein the symbol “ [symbol] ” denotes the point of attachment of a chemical moiety to the remainder of a molecule or chemical formula.

PEGs comprising one or more reactive functional groups may, in some embodiments, be obtained from, e.g., NOF America Corp. White Plains, NY or BOC Sciences 45-16 Ramsey Road Shirley, NY 11967, USA, among others, or may be prepared using methods known in the art.

In some embodiments, a linker is used to connect a compstatin analog described herein and a PEG described herein. Suitable linkers for connecting a compstatin analog and a PEG are extensively described above and in classes and subclasses herein. In some embodiments, a linker has multiple functional groups, wherein one functional group is connected to a compstatin analog and another is connected to a PEG moiety. In some embodiments, a linker is a bifunctional compound. In some embodiments, a linker has the structure of NH₂(CH₂CH₂O)nCH₂C(═O)OH, wherein n is 1 to 1000. In some embodiments, a linker is 8-amino-3,6-dioxaoctanoic acid (AEEAc). In some embodiments, a linker is activated for conjugation with a polymer moiety or a functional group of a compstatin analog. For example, in some embodiments, the carboxyl group of AEEAc is activated before conjugation with the amine group of the side chain of a lysine group.

In some embodiments, a suitable functional group (for example, an amine, hydroxyl, thiol, or carboxylic acid group) on a compstatin analog is used for conjugation with a PEG moiety, either directly or via a linker. In some embodiments, a compstatin analog is conjugated through an amine group to a PEG moiety via a linker. In some embodiments, an amine group is the α-amino group of an amino acid residue. In some embodiments, an amine group is the amine group of the lysine side chain. In some embodiments, a compstatin analog is conjugated to a PEG moiety through the amino group of a lysine side chain (ε-amino group) via a linker having the structure of NH₂(CH₂CH₂O)nCH₂C(═O)OH, wherein n is 1 to 1000. In some embodiments, a compstatin analog is conjugated to the PEG moiety through the amino group of a lysine side chain via an AEEAc linker. In some embodiments, the NH₂(CH₂CH₂O)nCH₂C(═O)OH linker introduces a —NH(CH₂CH₂O)nCH₂C(═O)— moiety on a compstatin lysine side chain after conjugation. In some embodiments, the AEEAc linker introduces a —NH(CH₂CH₂O)₂CH₂C(═O)— moiety on a compstatin lysine side chain after conjugation.

In some embodiments, a compstatin analog is conjugated to a PEG moiety via a linker, wherein the linker comprises an AEEAc moiety and an amino acid residue. In some embodiments, a compstatin analog is conjugated to a PEG moiety via a linker, wherein the linker comprises an AEEAc moiety and a lysine residue. In some embodiments, the C-terminus of a compstatin analog is connected to the amino group of AEEAc, and the C-terminus of AEEAc is connected to a lysine residue. In some embodiments, the C-terminus of a compstatin analog is connected to the amino group of AEEAc, and the C-terminus of AEEAc is connected to the α-amino group of a lysine residue. In some embodiments, the C-terminus of a compstatin analog is connected to the amino group of AEEAc, the C-terminus of AEEAc is connected to the α-amino group of the lysine residue, and a PEG moiety is conjugated through the ε-amino group of said lysine residue. In some embodiments, the C-terminus of the lysine residue is modified. In some embodiments, the C-terminus of the lysine residue is modified by amidation. In some embodiments, the N-terminus of a compstatin analog is modified. In some embodiments, the N-terminus of a compstatin analog is acetylated.

In certain embodiments, a compstatin analog may be represented as M-AEEAc-Lys-B₂, wherein B₂ is a blocking moiety, e.g., NH₂, M represents any of SEQ ID NOs: 9-36, with the proviso that the C-terminal amino acid of any of SEQ ID NOs: 9-36 is linked via a peptide bond to AEEAc-Lys-B₂. The NHS moiety of a monofunctional or multifunctional (e.g., bifunctional) PEG reacts with the free amine of the lysine side chain to generate a monofunctionalized (one compstatin analog moiety) or multifunctionalized (multiple compstatin analog moieties) PEGylated compstatin analog. In various embodiments, any amino acid comprising a side chain that comprises a reactive functional group may be used instead of Lys (or in addition to Lys). A monofunctional or multifunctional PEG comprising a suitable reactive functional group may be reacted with such side chain in a manner analogous to the reaction of NHS-ester activated PEGs with Lys.

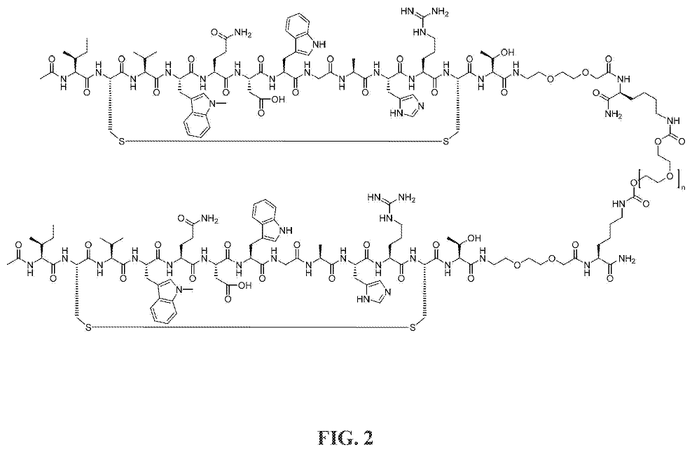

With regard to any of the above formulae and structures, it is to be understood that embodiments in which the compstatin analog component comprises any compstatin analog described herein, e.g., any compstatin analog of SEQ ID NOs; 9-36 are expressly disclosed. For example, and without limitation, a compstatin analog may comprise the amino acid sequence of SEQ ID NO: 28. An exemplary PEGylated compstatin analog in which the compstatin analog component comprises the amino acid sequence of SEQ ID NO: 28 is depicted in FIG. 2. It will be understood that the PEG moiety may have a variety of different molecular weights or average molecular weights in various embodiments, as described herein. In certain embodiments, a compstatin analog is pegcetacoplan (“APL-2”), having the structure of the compound of FIG. 2 with n of about 800 to about 1100 and a PEG having an average molecular weight of about 40 kD. Pegcetacoplan is also referred to as Poly(oxy-1,2-ethanediyl), α-hydro-ω-hydroxy-, 15,15′-diester with N-acetyl-L-isoleucyl-L-cysteinyl-L-valyl-1-methyl-L-tryptophyl-L-glutaminyl-L-α-aspartyl-L-tryptophylglycyl-L-alanyl-L-histidyl-L-arginyl-L-cysteinyl-L-threonyl-2-[2-(2-aminoethoxy)ethoxy]acetyl-N⁶-carboxy-L-lysinamide cyclic (2-->12)-(disulfide); or O,O′-bis[(S²,S¹²-cyclo{N-acetyl-L-isoleucyl-L-cysteinyl-L-valyl-1-methyl-L-tryptophyl-L-glutaminyl-L-α-aspartyl-L-tryptophylglycyl-L-alanyl-L-histidyl-L-arginyl-L-cysteinyl-L-threonyl-2-[2-(2-aminoethoxy)ethoxy]acetyl-L-lysinamide})-N^{6,15}-carbonyl]polyethylene glycol (n=800-1100). Additional compstatin analogs are described in, e.g., WO 2012/155107 and WO 2014/078731.

In some embodiments, a compstatin analog described herein is administered twice weekly or every 3 days, at a dosage of about 800 mg to about 1200 mg, e.g., about 1060 mg to about 1100 mg, e.g., about 1070 mg to about 1090 mg, e.g., about 1075 mg to about 1085 mg, e.g., about 1080 mg, for about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 1.2 years, 1.4 years, 1.6 years, 1.8 years, 2 years, 3 years, 4 years, 5 years, or longer.

In some embodiments, a composition comprising one or more inhibitory RNAs (e.g., an siRNA or miRNA described herein), or comprising a vector comprising a nucleotide sequence encoding an siRNA or miRNA described herein, is administered to a subject in combination with a compstatin analog, such that the compstatin analog and/or the inhibitory RNA composition is administered less frequently and/or at a lower dosage. In some embodiments, a composition comprising one or more inhibitory RNAs (e.g., an siRNA or miRNA described herein), or a vector comprising a nucleotide sequence encoding an siRNA or miRNA described herein, is administered to a subject in combination with a compstatin analog, such that the compstatin analog is administered once a week, once every 2 weeks, once a month, once every 2 months, 3 months, 4 months, 5 months, or longer, at a dosage of about 800 mg to about 1200 mg, e.g., about 1060 mg to about 1100 mg, e.g., about 1070 mg to about 1090 mg, e.g., about 1075 mg to about 1085 mg, e.g., about 1080 mg.

In some embodiments, a complement inhibitor is an antibody, e.g., an anti-factor B, an anti-C3 and/or anti-C5 antibody, or a fragment thereof. In some embodiments, an antibody fragment may be used to inhibit factor B, C3 or C5 activation. The fragmented anti-factor B, anti-C3 or anti-C5 antibody may be Fab′, Fab′ (2), Fv, or single chain Fv. In some embodiments, the anti-factor B, anti-C3 or anti-C5 antibody is monoclonal. In some embodiments, the anti-factor B, anti-C3 or anti-C5 antibody is polyclonal. In some embodiments, the anti-factor B, anti-C3 or anti-C5 antibody is de-immunized. In some embodiments the anti-factor B, anti-C3 or anti-C5 antibody is a fully human monoclonal antibody. In some embodiments, the anti-C5 antibody is eculizumab.

In some embodiments, a complement inhibitor is a polypeptide inhibitor and/or a nucleic acid aptamer (see, e.g., U.S. Publ. No. 20030191084). Exemplary polypeptide inhibitors include an enzyme that degrades C3 or C3b (see, e.g., U.S. Pat. No. 6,676,943). Additional polypeptide inhibitors include mini-factor H (see, e.g., U.S. Publ. No. 20150110766), Efb protein or complement inhibitor (SCIN) protein from Staphylococcus aureus, or a variant or derivative or mimetic thereof (see, e.g., U.S. Publ. 20140371133).

A variety of other complement inhibitors can also be used in various embodiments of the disclosure. In some embodiments, the complement inhibitor is a naturally occurring mammalian complement regulatory protein or a fragment or derivative thereof. For example, the complement regulatory protein may be CR1, DAF, MCP, CFH, or CFI. In some embodiments, the complement regulatory polypeptide is one that is normally membrane-bound in its naturally occurring state. In some embodiments, a fragment of such polypeptide that lacks some or all of a transmembrane and/or intracellular domain is used. Soluble forms of complement receptor 1 (sCR1), for example, can also be used. For example the compounds known as TP10 or TP20 (Avant Therapeutics) can be used. C1 inhibitor (C1-INH) can also be used. In some embodiments a soluble complement control protein, e.g., CFH, is used.

Inhibitors of C1s can also be used. For example, U.S. Pat. No. 6,515,002 describes compounds (furanyl and thienyl amidines, heterocyclic amidines, and guanidines) that inhibit C1s. U.S. Pat. Nos. 6,515,002 and 7,138,530 describe heterocyclic amidines that inhibit C1s. U.S. Pat. No. 7,049,282 describes peptides that inhibit classical pathway activation. Certain of the peptides comprise or consist of WESNGQPENN (SEQ ID NO: 73) or KTISKAKGQPREPQVYT (SEQ ID NO: 74) or a peptide having significant sequence identity and/or three-dimensional structural similarity thereto. In some embodiments these peptides are identical or substantially identical to a portion of an IgG or IgM molecule. U.S. Pat. No. 7,041,796 discloses C3b/C4b Complement Receptor-like molecules and uses thereof to inhibit complement activation. U.S. Pat. No. 6,998,468 discloses anti-C2/C2a inhibitors of complement activation. U.S. Pat. No. 6,676,943 discloses human complement C3-degrading protein from Streptococcus pneumoniae.

All publications, patent applications, patents, and other references mentioned herein, including GenBank Accession Numbers, are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

The disclosure is further illustrated by the following examples. The examples are provided for illustrative purposes only. They are not to be construed as limiting the scope or content of the disclosure in any way.

### XI. Exemplification

### Example 1: Knockdown of Factor B Expression in HepG2 Cells Using siRNAs

### Cell Culture

HepG2 cells were obtained from ATCC (ATCC in partnership with LGC Standards, Wesel, Germany, cat. #ATCC-HB-8065) and cultured in MEM Eagle (#M2279, Sigma-Aldrich, Germany), supplemented to contain 10% fetal calf serum (#1248D, Biochrom GmbH, Berlin, Germany), 1×non-essential amino acids (#K0293; Biochrom, Berlin, Germany), 4 mM L-Glutamine (#K0283, Biochrom, Berlin, Germany) and 100 U/ml Penicillin/100 μg/ml Streptomycin (#A2213, Biochrom GmbH, Berlin, Germany) at 37° C. in an atmosphere with 5% CO2 in a humidified incubator.

siRNAs

24 siRNAs were designed and synthesized to target different regions of the factor B mRNA transcript. In this experiment, the sense strand of each siRNA contained 18 nucleotides identical to a target region sequence on the factor B transcript (SEQ ID NO: 75), and one additional adenine nucleotide at the 3′ end. Additionally, in this experiment, the antisense strand contained 18 nucleotides complementary to a target region sequence on the factor B transcript (SEQ ID NO: 75), and one additional uracil nucleotide at the 5′ end, and 2 additional uracil nucleotides at the 3′ end.

In this experiment, siRNAs contained modifications of the sense strand that included the following modification pattern:

xsxsXfxxxXfXfXfxxXfxxxxXfxa

Antisense strands included the following modification pattern:

| | |
|---|---|
| usXfsxxxxXX | xxxxXfxxxxxsusu |

where “x” represents any nucleotide; a lowercase letter represents a nucleotide modified with a 2′-O-Methyl group; “Xf” represents a nucleotide (“X” can be any nucleotide) modified with a 2′-Fluoro group. For example, “Af” represents an adenine nucleotide modified with a 2′-Fluoro group. An “s” represents a phosphorothioate bond.

### Dose Response Experiments

Transfection of siRNA was carried out with Lipofectamine RNAiMax (Invitrogen/Life Technologies, Karlsruhe, Germany) according to manufacturer's instructions for reverse transfection.

Each siRNA was tested using dose response experiments (DRC) in HepG2 cells. Dose-response experiments were done with siRNA in 10 concentrations transfected in quadruplicates, starting at 100 nM in 6-fold dilutions steps down to ˜10 fM. Mock transfected cells served as negative controls.

For each well, the target mRNA level was normalized to the respective GAPDH mRNA level. The activity of a given siRNA was expressed as percent mRNA concentration of the respective target (normalized to GAPDH mRNA) in treated cells, relative to the target mRNA concentration (normalized to GAPDH mRNA) averaged across mock transfected wells (DRCs).

IC20, IC50, IC80 values, and Max inhibition (%) from the DRC experiments are shown in Table 8 below. The sequences for these siRNAs are shown in Table 9 below.

TABLE 8

| | | | | Max Factor B |
|---|---|---|---|---|
| siRNA ID | IC20 [nm] | IC₅₀ [nM] | IC₈₀ [nM] | inhibition (%) |
| 1 | 0.03 | 0.54 | 19.84 | 91.04 |
| 2 | 0.00 | 0.00 | 7.07 | 90.18 |
| 3 | 0.02 | 0.29 | 12.01 | 89.76 |
| 4 | 0.05 | 0.48 | 10.94 | 88.29 |
| 5 | 0.10 | 0.67 | 9.65 | 86.90 |
| 6 | 0.03 | 0.37 | 16.08 | 85.34 |
| 7 | 0.00 | 0.02 | 669.67 | 82.44 |
| 8 | 0.00 | 0.16 | 717.07 | 82.24 |
| 9 | 0.01 | 0.27 | 142.09 | 82.07 |
| 10 | 0.43 | 1.70 | 31.62 | 81.72 |
| 11 | 0.01 | 0.26 | 2348.78 | 81.08 |
| 12 | 0.13 | 0.89 | 183.20 | 80.54 |
| 13 | 0.06 | 0.69 | 1336.57 | 80.41 |
| 14 | 0.22 | 1.30 | #N/A | 79.20 |
| 15 | 1.70 | 6.31 | #N/A | 78.63 |
| 16 | 0.01 | 0.30 | #N/A | 77.05 |
| 17 | 0.07 | 0.85 | #N/A | 75.50 |
| 18 | 0.30 | 2.02 | #N/A | 75.45 |
| 19 | 0.16 | 1.12 | #N/A | 74.03 |
| 20 | 1.10 | 3.52 | #N/A | 74.02 |
| 21 | 0.12 | 1.63 | #N/A | 72.43 |
| 22 | 0.32 | 2.09 | #N/A | 72.01 |
| 23 | 0.22 | 2.44 | #N/A | 68.74 |
| 24 | 0.51 | 2.76 | #N/A | 61.56 |

TABLE 9

| SiRNA | Sense sequence | Antisense sequence |
|---|---|---|
| ID | (5′ to 3′) | (5′ to 3′) |
| 1 | cscsUfacuAfCfAfau | usCfsacucacauugu |
| | GfugagUfga | Afguaggsusu |
| | (SEQ ID NO: 220) | (SEQ ID NO: 221) |
| 2 | csusUfcugGfCfUfuc | usAfscggguagaagc |
| | UfacccGfua | Cfagaagsusu |
| | (SEQ ID NO: 222) | (SEQ ID NO: 223) |
| 3 | ususCfuggCfUfUfcu | usUfsacggguagaag |
| | AfcccgUfaa | Cfcagaasusu |
| | (SEQ ID NO: 224) | (SEQ ID NO: 225) |
| 4 | csusGfcuaUfGfAfcg | usGfsuguaaccguca |
| | GfuuacAfca | Ufagcagsusu |
| | (SEQ ID NO: 226) | (SEQ ID NO: 227) |
| 5 | gsgsGfgagUfAfGfug | usAfsgacauccacua |
| | GfauguCfua | Cfuccccsusu |
| | (SEQ ID NO: 228) | (SEQ ID NO: 229) |
| 6 | usgsUfgguGfUfCfug | usAfsaguacucagac |
| | AfguacUfua | Afccacasusu |
| | (SEQ ID NO: 230) | (SEQ ID NO: 231) |
| 7 | usasCfgugUfGfUfcc | usGfsccagaaggaca |
| | UfucugGfca | Cfacguasusu |
| | (SEQ ID NO: 232) | (SEQ ID NO: 233) |
| 8 | asasGfuguCfUfAfgu | usUfsaaguugacuag |
| | CfaacuUfaa | Afcacuususu |
| | (SEQ ID NO: 234) | (SEQ ID NO: 235) |
| 9 | csusAfugaCfGfUfug | usAfsucagggcaacg |
| | CfccugAfua | Ufcauagsusu |
| | (SEQ ID NO: 236) | (SEQ ID NO: 237) |
| 10 | csusCfgguUfCfCfuu | usCfsaguacaaagga |
| | UfguacUfga | Afccgagsusu |
| | (SEQ ID NO: 238) | (SEQ ID NO: 239) |
| 11 | ascsCfauaGfAfAfgg | usAfsucgacuccuuc |
| | AfgucgAfua | Ufauggususu |
| | (SEQ ID NO: 240) | (SEQ ID NO: 241) |
| 12 | gsgsAfuuaUfCfUfgg | usUfsagacauccaga |
| | AfugucUfaa | Ufaauccsusu |
| | (SEQ ID NO: 242) | (SEQ ID NO: 243) |
| 13 | ascsGfuugCfCfCfug | usGfscuugaucaggg |
| | AfucaaGfca | Cfaacgususu |
| | (SEQ ID NO: 244) | (SEQ ID NO: 245) |
| 14 | gsgsAfgccAfAfAfaa | usUfsagacacuuuuu |
| | GfugucUfaa | Gfgcuccsusu |
| | (SEQ ID NO: 246) | (SEQ ID NO: 247) |
| 15 | cscsGfauuAfCfCfac | usGfsuugcuuguggu |
| | AfagcaAfca | Afaucggsusu |
| | (SEQ ID NO: 248) | (SEQ ID NO: 249) |
| 16 | asasUfgcaGfAfCfug | usCfsgugacccaguc |
| | GfgucaCfga | Ufgcauususu |
| | (SEQ ID NO: 250) | (SEQ ID NO: 251) |
| 17 | csusCfacgCfCfCfga | usGfsaaagucucggg |
| | GfacuuUfca | Cfgugagsusu |
| | (SEQ ID NO: 252) | (SEQ ID NO: 253) |
| 18 | csasGfcgaUfCfUfgu | usCfsguugucacaga |
| | GfacaaCfga | Ufcgcugsusu |
| | (SEQ ID NO: 254) | (SEQ ID NO: 255) |
| 19 | csusAfccaCfUfUfgc | usGfsuugcuggcaag |
| | CfagcaAfca | Ufgguagsusu |
| | (SEQ ID NO: 256) | (SEQ ID NO: 257) |
| 20 | ususUfaugAfCfUfau | usCfsaacgucauagu |
| | GfacguUfga | Cfauaaasusu |
| | (SEQ ID NO: 258) | (SEQ ID NO: 259) |
| 21 | cscsUfugaUfAfGfuu | usUfscuugugaacua |
| | CfacaaGfaa | Ufcaaggsusu |
| | (SEQ ID NO: 260) | (SEQ ID NO: 261) |
| 22 | csusCfaagAfGfGfug | usCfsuucggccaccu |
| | GfccgaAfga | Cfuugagsusu |
| | (SEQ ID NO: 262) | (SEQ ID NO: 263) |
| 23 | ususUfuauGfAfCfua | usAfsacgucauaguc |
| | UfgacgUfua | Afuaaaasusu |
| | (SEQ ID NO: 264) | (SEQ ID NO: 265) |
| 24 | ususCfcgaCfUfUfcu | usCfsucuuggagaag |
| | CfcaagAfga | Ufcggaasusu |
| | (SEQ ID NO: 266) | (SEQ ID NO: 267) |

### Additional Dose Response Experiments

Dose response experiments described above were repeated in a subset of siRNAs shown in Table 9, specifically siRNAs 2, 7, 8, and 3.

IC20, IC50, IC80 values, and Max inhibition (%) from the DRC experiments are shown in Table 10 below.

TABLE 10

| siRNA ID | IC20 [nM] | IC50 [nM] | IC80 [nM] | Max KD [%] |
|---|---|---|---|---|
| 2 | 0.021 | 0.0887 | 0.8652 | 83.5 |
| 7 | 0.026 | 0.1028 | 0.8678 | 83.5 |
| 8 | 0.0889 | 0.2713 | #N/A | 76.2 |
| 3 | 0.0722 | 0.2043 | 0.8863 | 87 |

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

We claim:

1. An siRNA comprising an antisense strand and a sense strand, wherein the antisense strand is complementary to a nucleotide sequence that is at least 90% identical to any one of SEQ ID NOs: 76-99 and/or the sense strand comprises a nucleotide sequence that is at least 90% identical to any one of SEQ ID NOs: 76-99.

2. An siRNA comprising an antisense strand and a sense strand, wherein the antisense strand is complementary to a nucleotide sequence comprising a sequence that differs by no more than 1, 2, 3, or 4 nucleotides from any one of SEQ ID NOs: 76-99 and/or the sense strand comprises a nucleotide sequence that differs by no more than 1, 2, 3, or 4 nucleotides from any one of SEQ ID NOs: 76-99.

3. The siRNA of claim 1 or claim 2, wherein the antisense strand is complementary to a nucleotide sequence comprising any one of SEQ ID NOs: 76-99.

4. The siRNA of any one of claims 1-3, wherein the antisense strand comprises a nucleotide sequence comprising any one of SEQ ID NOs: 100-123.

5. The siRNA of any one of claims 1-4, wherein one or both of the sense strand and the antisense strand comprises at least one overhang region.

6. The siRNA of claim 5, wherein the at least one overhang comprises a 1, 2, 3, 4, or 5, nucleotide overhang.

7. The siRNA of claim 5 or 6, wherein the at least one overhang comprises a 3′ overhang.

8. The siRNA of any claim 6 or 7, wherein the overhang region is complementary to a fragment of SEQ ID NO: 75.

9. The siRNA of claim 7 or 8, wherein the 3′ overhang comprises a 2-nucleotide overhang.

10. The siRNA of any one of claims 1-9, wherein one or both of the sense strand and the antisense strand comprises at least one additional nucleotide on the 5′ end, the 3′ end, or both the 5′ end and the 3′ end, which is not complementary to a fragment of SEQ ID NO: 75.

11. The siRNA of any one of claims 1-10, wherein one or both of the sense stand and the antisense strand comprises at least one modified nucleotide.

12. The siRNA of claim 11, wherein the at least one modified nucleotide comprises a nucleotide that includes a 2′-O-Methyl group, a nucleotide that includes a 2′-Fluoro group, and/or a phosphorothioate bond with an adjacent nucleotide.

13. The siRNA of claim 12, wherein the at least one modified nucleotide comprises a phosphorothioate bond between the last two, three, or four nucleotides of (i) the 5′ terminus of the sense strand; (ii) the 3′ terminus of the sense strand; (iii) the 5′ terminus of the antisense strand, and/or (iv) the 3′ terminus of the antisense strand.

14. The siRNA of claim 13, wherein the at least one modified nucleotide comprises a phosphorothioate bond between the last three nucleotides of (i) the 5′ terminus of the sense strand; (ii) the 3′ terminus of the sense strand; (iii) the 5′ terminus of the antisense strand, and/or (iv) the 3′ terminus of the antisense strand.

15. The siRNA of claim 13, wherein the at least one modified nucleotide comprises a phosphorothioate bond between the last two, three, or four nucleotides of (i) the 5′ terminus of the sense strand; (ii) the 3′ terminus of the sense strand; (iii) the 5′ terminus of the antisense strand, and (iv) the 3′ terminus of the antisense strand.

16. The siRNA of any one of claims 1-15, wherein the sense strand comprises the nucleotide sequence of any one of SEQ ID NOs: 76-99, 124-147, 220, 222, 224, 226, 228, 230, 232, 234, 236, 238, 240, 242, 244, 246, 248, 250, 252, 254, 256, 258, 260, 262, 264, and 266.

17. The siRNA of any one of claims 1-16, wherein the antisense strand comprises the nucleotide sequence of any one of SEQ ID NOs: 100-123, 148-219, 221, 223, 225, 227, 229, 231, 233, 235, 237, 239, 241, 243, 245, 247, 249, 251, 253, 255, 257, 259, 261, 263, 265, and 267.

18. The siRNA of any one of claims 1-17, comprising a sense strand nucleotide sequence/antisense strand nucleotide sequence of any one of the following sets of sense/antisense SEQ ID NOs: 220/221, 222/223, 224/225, 226/227, 228/229, 230/231, 232/233, 234/235, 236/237, 238/239, 240/241, 242/243, 244/245, 246/247, 248/249, 250/251, 252/253, 254/255, 256/257, 258/259, 260/261, 262/263, 264/265, and 266/267.

19. The siRNA of any one of claims 1-18, further comprising at least one ligand attached to one or more of the 5′ end of the sense strand, the 3′ end of the sense strand, the 5′ end of the antisense strand, and the 3′ end of the antisense strand.

20. The siRNA of claim 19, wherein the ligand comprises at least one GalNAc moiety.

21. The siRNA of claim 20, wherein the ligand comprises three GalNAc moieties.

22. A method of treating a subject having or at risk of a complement-mediated disorder, the method comprising administering to the subject a composition comprising an effective amount of the siRNA of any one of claims 1-21.

23. The method of claim 22, comprising administering to the subject a composition comprising a nucleic acid encoding the siRNA of any one of claims 1-18.

24. The method of claim 22 or 23, wherein after the administration of the composition, a level of factor B transcript or factor B protein in the subject or in a biological sample from the subject is reduced relative to a level before the administration of the composition.

25. The method of claim 24, wherein the level of factor B transcript or factor B protein is reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90%, relative to a level before the administration.

26. The method of any one of claims 22-25, wherein the composition is administered intravenously or subcutaneously to the subject.

27. The method of any one of claims 22-26, wherein the composition is administered to a hepatocyte of the subject.

28. The method of claim 27, wherein the composition is administered to the hepatocyte ex vivo.

29. The method of claim 27, wherein the composition is administered to the hepatocyte in vivo.

30. The method of any one of claims 22-29, further comprising administering to the subject a second agent.

31. The method of claim 30, wherein the second agent is an anti-factor B antibody or a compstatin analog.

32. The method of any one of claims 22-31, wherein the subject has a defect in complement regulation, optionally wherein the defect comprises abnormally low expression of one or more complement regulatory proteins by at least some of the subject's cells.

33. The method of any one of claims 22-32, wherein the complement-mediated disorder is a chronic disorder.

34. The method of any one of claims 22-33, wherein the complement-mediated disorder involves complement-mediated damage to red blood cells, optionally wherein the disorder is paroxysmal nocturnal hemoglobinuria or atypical hemolytic uremic syndrome.

35. The method of any one of claims 22-34, wherein the complement-mediated disorder is an autoimmune disease, optionally wherein the disorder is multiple sclerosis.

36. The method of any one of claims 22-35, wherein the complement-mediated disorder involves the kidney, optionally wherein the disorder is membranoproliferative glomerulonephritis, lupus nephritis, IgA nephropathy (IgAN), primary membranous nephropathy (primary MN), C3 glomerulopathy (C3G), or acute kidney injury.

37. The method of any one of claims 22-36, wherein the complement-mediated disorder involves the central or peripheral nervous system or neuromuscular junction, optionally wherein the disorder is neuromyelitis optica, Guillain-Barré syndrome, multifocal motor neuropathy, or myasthenia gravis.

38. A composition comprising the siRNA of any one of claims 1-21 and a carrier and/or excipient.

39. An expression vector comprising one or more nucleotide sequences encoding one or more siRNAs of any one of claims 1-18.

40. The expression vector of claim 39, further comprising a nucleotide sequence encoding a factor B inhibitor (e.g., an aptamer, an anti-factor B antibody, a mammalian complement regulatory protein, or mini factor H).

41. A composition comprising:
(i) a sense strand comprising the nucleotide sequence of any one of SEQ ID NOs: 76-99, 124-147, 220, 222, 224, 226, 228, 230, 232, 234, 236, 238, 240, 242, 244, 246, 248, 250, 252, 254, 256, 258, 260, 262, 264, and 266; and
(ii) an antisense strand comprising the nucleotide sequence of any one of SEQ ID NOs: 100-123, 148-219, 221, 223, 225, 227, 229, 231, 233, 235, 237, 239, 241, 243, 245, 247, 249, 251, 253, 255, 257, 259, 261, 263, 265, and 267.

42. An antisense nucleic acid comprising the nucleotide sequence of any one of SEQ ID NOs: 100-123, 148-219, 221, 223, 225, 227, 229, 231, 233, 235, 237, 239, 241, 243, 245, 247, 249, 251, 253, 255, 257, 259, 261, 263, 265, and 267.

43. A method of reducing or inhibiting complement factor B expression in a cell, the method comprising contacting the cell with the siRNA of any one of claims 1-21, the composition of claim 38 or 41, the vector of claim 39 or 40, or the antisense nucleic acid of claim 42.

44. The method of claim 43, wherein after the contacting step, the level of factor B transcript or factor B protein is reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90%, relative to a level before the contacting step.

45. The method of claim 43 or 44, wherein the cell is in a subject.

46. The method of any one of claim 22-37 or 45, wherein the subject is a human.

47. The method of claim 46, wherein the subject suffers from a complement-mediated disorder.

48. A method of reducing or inhibiting expression of factor B in a subject, the method comprising contacting a cell of the subject with the siRNA of any one of claims 1-21, the composition of claim 38 or 41, the vector of claim 39 or 40, or the antisense nucleic acid of claim 42.

49. The method of claim 48, wherein after the contacting step, the level of factor B transcript or factor B protein is reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90%, relative to a level before the contacting step.

50. The method of claim 48 or 49, wherein the subject is a human.

51. The method of claim 50, wherein the subject suffers from a complement-mediated disorder.

52. A method of reducing or inhibiting expression of factor B in a subject, the method comprising administering to the subject the siRNA of any one of claims 1-21, the composition of claim 38 or 41, the vector of claim 39 or 40, or the antisense nucleic acid of claim 42.

53. The method of claim 52, wherein after the administering step, the level of factor B transcript or factor B protein is reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90%, relative to a level before the administering step.

54. The method of claim 52 or 53, wherein the subject is a human.

55. The method of claim 54, wherein the subject suffers from a complement-mediated disorder.


SEQUENCE LISTING (268 sequences)

SEQ ID NO: 1 - protein, 13 aa, synthetic construct
  DISULFID 2..12: note = Intrachain disulfide bond
ICVVQDWGHHRCT

SEQ ID NO: 2-7 - skipped (6 entries)

SEQ ID NO: 8 - protein, 13 aa, synthetic construct
  SITE 1: note = N-term H - hydrogenated Isoleucine
  SITE 13: note = C-term CONH2 - Threonine amide
ICVVQDWGHHRCT

SEQ ID NO: 9 - protein, 13 aa, synthetic construct
  SITE 1: note = N-term Ac - acetyl Isoleucine
  SITE 13: note = C-term CONH2 - Threonine amide
ICVVQDWGHHRCT

SEQ ID NO: 10 - protein, 13 aa, synthetic construct
  SITE 1: note = N-term Ac - acetyl Isoleucine
  SITE 13: note = C-term CONH2 - Threonine amide
ICVYQDWGAHRCT

SEQ ID NO: 11 - protein, 13 aa, synthetic construct
  SITE 1: note = N-term Ac - acetyl Isoleucine
  SITE 13: note = C-term COOH - Threonine carboxide
ICVWQDWGAHRCT

SEQ ID NO: 12 - protein, 13 aa, synthetic construct
  SITE 1: note = N-term Ac - acetyl Isoleucine
  SITE 13: note = C-term CONH2 - Threonine amide
ICVWQDWGAHRCT

SEQ ID NO: 13 - protein, 13 aa, synthetic construct
  SITE 1: note = N-term Ac - acetyl Isoleucine
  SITE 13: note = C-term COOH - D-Threonine carboxide
ICVWQDWGAHRCT

SEQ ID NO: 14 - protein, 13 aa, synthetic construct
  SITE 1: note = N-term Ac - acetyl Isoleucine
  SITE 4: note = 2-Naphthylalanine
  SITE 13: note = C-term CONH2 - Threonine amide
ICVXQDWGAHRCT

SEQ ID NO: 15 - protein, 13 aa, synthetic construct
  SITE 1: note = N-term Ac - acetyl Isoleucine
  SITE 4: note = 2-Naphthylalanine
  SITE 13: note = C-term COOH - Threonine carboxide
ICVXQDWGAHRCT

SEQ ID NO: 16 - protein, 13 aa, synthetic construct
  SITE 1: note = N-term Ac - acetyl Isoleucine
  SITE 4: note = 1-Naphthylalanine
  SITE 13: note = C-term COOH - Threonine carboxide
ICVXQDWGAHRCT

SEQ ID NO: 17 - protein, 13 aa, synthetic construct
  SITE 1: note = N-term Ac - acetyl Isoleucine
  SITE 4: note = 2-Indanylglycine
  SITE 13: note = C-term CONH2 - Threonine amide
ICVXQDWGAHRCT

SEQ ID NO: 18 - protein, 13 aa, synthetic construct
  SITE 1: note = N-term Ac - acetyl Isoleucine
  SITE 4: note = 2-Indanylglycine
  SITE 13: note = C-term COOH - Threonine carboxide
ICVXQDWGAHRCT

SEQ ID NO: 19 - protein, 13 aa, synthetic construct
  SITE 1: note = N-term Ac - acetyl Isoleucine
  SITE 4: note = dihydrotryptophan
  SITE 13: note = C-term COOH - Threonine carboxide
ICVXQDWGAHRCT

SEQ ID NO: 20 - protein, 13 aa, synthetic construct
  SITE 1: note = N-term Ac - acetyl Isoleucine
  SITE 4: note = 4-Benzoylphenylalanine
  SITE 13: note = C-term COOH - Threonine carboxide
ICVXQDWGAHRCT

SEQ ID NO: 21 - protein, 13 aa, synthetic construct
  SITE 1: note = N-term Ac - acetyl Isoleucine
  SITE 4: note = 4-Benzoylphenylalanine
ICVXQDWGAHRCT

SEQ ID NO: 22 - protein, 13 aa, synthetic construct
  SITE 1: note = N-term Ac - acetyl Isoleucine
  SITE 4: note = beta-3-benzothienyl-L-alanine
  SITE 13: note = C-term COOH - Threonine carboxide
ICVXQDWGAHRCT

SEQ ID NO: 23 - protein, 13 aa, synthetic construct
  SITE 1: note = N-term Ac - acetyl Isoleucine
  SITE 4: note = beta-3-benzothienyl-L-alanine
ICVXQDWGAHRCT

SEQ ID NO: 24 - protein, 13 aa, synthetic construct
  SITE 1: note = N-term Ac - acetyl Isoleucine
  SITE 9: note = 2-alpha-aminobutyric acid
  SITE 13: note = C-term CONH2 - Threonine amide
ICVWQDWGXHRCT

SEQ ID NO: 25 - protein, 16 aa, synthetic construct
  SITE 1: note = N-term H - hydrogenated Glycine
  SITE 16: note = C-term COOH - Asparagine carboxide
GICVWQDWGAHRCTAN

SEQ ID NO: 26 - protein, 13 aa, synthetic construct
  SITE 1: note = N-term Ac - acetyl Isoleucine
  SITE 4: note = 5-fluoro-tryptophan
  SITE 13: note = C-term CONH2 - Threonine amide
ICVXQDWGAHRCT

SEQ ID NO: 27 - protein, 13 aa, synthetic construct
  SITE 1: note = N-term Ac - acetyl Isoleucine
  SITE 4: note = 5-methyl-tryptophan
  SITE 13: note = C-term CONH2 - Threonine amide
ICVXQDWGAHRCT

SEQ ID NO: 28 - protein, 13 aa, synthetic construct
  SITE 1: note = N-term Ac - acetyl Isoleucine
  SITE 4: note = 1-methyl-tryptophan
  SITE 13: note = C-term CONH2 - Threonine amide
ICVXQDWGAHRCT

SEQ ID NO: 29 - protein, 13 aa, synthetic construct
  SITE 1: note = N-term Ac - acetyl Isoleucine
  SITE 7: note = 5-fluoro-tryptophan
  SITE 13: note = C-term CONH2 - Threonine amide
ICVWQDXGAHRCT

SEQ ID NO: 30 - protein, 13 aa, synthetic construct
  SITE 1: note = N-term Ac - acetyl Isoleucine
  SITE 4: note = 5-fluoro-tryptophan
  SITE 7: note = 5-fluoro-tryptophan
  SITE 13: note = C-term CONH2 - Threonine amide
ICVXQDXGAHRCT

SEQ ID NO: 31 - protein, 13 aa, synthetic construct
  SITE 1: note = N-term Ac - acetyl Isoleucine
  SITE 4: note = 5-methyl-tryptophan
  SITE 7: note = 5-fluoro-tryptophan
  SITE 13: note = C-term CONH2 - Threonine amide
ICVXQDXGAHRCT

SEQ ID NO: 32 - protein, 13 aa, synthetic construct
  SITE 1: note = N-term Ac - acetyl Isoleucine
  SITE 4: note = 1-methyl-tryptophan
  SITE 7: note = 5-fluoro-tryptophan
  SITE 13: note = C-term CONH2 - Threonine amide
ICVXQDXGAHRCT

SEQ ID NO: 33 - protein, 15 aa, synthetic construct
  SITE 1: note = N-term H - hydrogenated Glycine
  SITE 5: note = 6-fluoro-tryptophan
  SITE 8: note = 6-fluoro-tryptophan
  SITE 15: note = C-term COOH - Asparagine carboxide
GICVXQDXGAHRCTN

SEQ ID NO: 34 - protein, 13 aa, synthetic construct
  SITE 1: note = N-term Ac - acetyl Isoleucine
  SITE 4: note = 1-formyl-Tryptophan
  SITE 13: note = C-term CONH2 - Threonine amide
ICVXQDWGAHRCT

SEQ ID NO: 35 - protein, 13 aa, synthetic construct
  SITE 1: note = N-term Ac - acetyl Isoleucine
  SITE 4: note = 1-methyoxy-Tryptophan
  SITE 13: note = C-term CONH2 - Threonine amide
ICVXQDWGAHRCT

SEQ ID NO: 36 - protein, 15 aa, synthetic construct
  SITE 1: note = N-term H - hydrogenated Glycine
  SITE 5: note = 5-fluoro-tryptophan
  SITE 8: note = 5-fluoro-tryptophan
  SITE 15: note = C-term COOH - asparagine carboxide
GICVXQDXGAHRCTN

SEQ ID NO: 37-72 - skipped (36 entries)

SEQ ID NO: 73 - protein, 10 aa, unidentified
WESNGQPENN

SEQ ID NO: 74 - protein, 17 aa, unidentified
KTISKAKGQPREPQVYT

SEQ ID NO: 75 - RNA, 2476 nt, Homo sapiens
  source 1..2476: mol_type = genomic RNA
gggaagggaatgtgaccaggtctaggtctggagtttcagcttggacactgagccaagcagacaagcaaagcaagccaggacacaccatcctgccccaggcccagcttctctcctgccttccaacgccatggggagcaatctcagcccccaactctgcctgatgccctttatcttgggcctcttgtctggaggtgtgaccaccactccatggtctttggcccggccccagggatcctgctctctggagggggtagagatcaaaggcggctccttccgacttctccaagagggccaggcactggagtacgtgtgtccttctggcttctacccgtaccctgtgcagacacgtacctgcagatctacggggtcctggagcaccctgaagactcaagaccaaaagactgtcaggaaggcagagtgcagagcaatccactgtccaagaccacacgacttcgagaacggggaatactggccccggtctccctactacaatgtgagtgatgagatctctttccactgctatgacggttacactctccggggctctgccaatcgcacctgccaagtgaatggccgatggagtgggcagacagcgatctgtgacaacggagcggggtactgctccaacccgggcatccccattggcacaaggaaggtgggcagccagtaccgccttgaagacagcgtcacctaccactgcagccgggggcttaccctgcgtggctcccagcggcgaacgtgtcaggaaggtggctcttggagcgggacggagccttcctgccaagactccttcatgtacgacacccctcaagaggtggccgaagctttcctgtcttccctgacagagaccatagaaggagtcgatgctgaggatgggcacggcccaggggaacaacagaagcggaagatcgtcctggacccttcaggctccatgaacatctacctggtgctagatggatcagacagcattggggccagcaacttcacaggagccaaaaagtgtctagtcaacttaattgagaaggtggcaagttatggtgtgaagccaagatatggtctagtgacatatgccacataccccaaaatttgggtcaaagtgtctgaagcagacagcagtaatgcagactgggtcacgaagcagctcaatgaaatcaattatgaagaccacaagttgaagtcagggactaacaccaagaaggccctccaggcagtgtacagcatgatgagctggccagatgacgtccctcctgaaggctggaaccgcacccgccatgtcatcatcctcatgactgatggattgcacaacatgggcggggacccaattactgtcattgatgagatccgggacttgctatacattggcaaggatcgcaaaaacccaagggaggattatctggatgtctatgtgtttggggtcgggcctttggtgaaccaagtgaacatcaatgctttggcttccaagaaagacaatgagcaacatgtgttcaaagtcaaggatatggaaaacctggaagatgttttctaccaaatgatcgatgaaagccagtctctgagtctctgtggcatggtttgggaacacaggaagggtaccgattaccacaagcaaccatggcaggccaagatctcagtcattcgcccttcaaagggacacgagagctgtatgggggctgtggtgtctgagtactttgtgctgacagcagcacattgtttcactgtggatgacaaggaacactcaatcaaggtcagcgtaggaggggagaagcgggacctggagatagaagtagtcctatttcaccccaactacaacattaatgggaaaaaagaagcaggaattcctgaattttatgactatgacgttgccctgatcaagctcaagaataagctgaaatatggccagactatcaggcccatttgtctcccctgcaccgagggaacaactcgagctttgaggcttcctccaactaccacttgccagcaacaaaaggaagagctgctccctgcacaggatatcaaagctctgtttgtgtctgaggaggagaaaaagctgactcggaaggaggtctacatcaagaatggggataagaaaggcagctgtgagagagatgctcaatatgccccaggctatgacaaagtcaaggacatctcagaggtggtcacccctcggttcctttgtactggaggagtgagtccctatgctgaccccaatacttgcagaggtgattctggcggccccttgatagttcacaagagaagtcgtttcattcaagttggtgtaatcagctggggagtagtggatgtctgcaaaaaccagaagcggcaaaagcaggtacctgctcacgcccgagactttcacatcaacctctttcaagtgctgccctggctgaaggagaaactccaagatgaggatttgggttttctataaggggtttcctgctggacaggggcgtgggattgaattaaaacagctgcgacaaca

SEQ ID NO: 76 - RNA, 18 nt, Homo sapiens
  source 1..18: mol_type = genomic RNA
cctactacaatgtgagtg

SEQ ID NO: 77 - RNA, 18 nt, Homo sapiens
  source 1..18: mol_type = genomic RNA
cttctggcttctacccgt

SEQ ID NO: 78 - RNA, 18 nt, Homo sapiens
  source 1..18: mol_type = genomic RNA
ttctggcttctacccgta

SEQ ID NO: 79 - RNA, 18 nt, Homo sapiens
  source 1..18: mol_type = genomic RNA
ctgctatgacggttacac

SEQ ID NO: 80 - RNA, 18 nt, Homo sapiens
  source 1..18: mol_type = genomic RNA
ggggagtagtggatgtct

SEQ ID NO: 81 - RNA, 18 nt, Homo sapiens
  source 1..18: mol_type = genomic RNA
tgtggtgtctgagtactt

SEQ ID NO: 82 - RNA, 18 nt, Homo sapiens
  source 1..18: mol_type = genomic RNA
tacgtgtgtccttctggc

SEQ ID NO: 83 - RNA, 18 nt, Homo sapiens
  source 1..18: mol_type = genomic RNA
aagtgtctagtcaactta

SEQ ID NO: 84 - RNA, 18 nt, Homo sapiens
  source 1..18: mol_type = genomic RNA
ctatgacgttgccctgat

SEQ ID NO: 85 - RNA, 18 nt, Homo sapiens
  source 1..18: mol_type = genomic RNA
ctcggttcctttgtactg

SEQ ID NO: 86 - RNA, 18 nt, Homo sapiens
  source 1..18: mol_type = genomic RNA
accatagaaggagtcgat

SEQ ID NO: 87 - RNA, 18 nt, Homo sapiens
  source 1..18: mol_type = genomic RNA
ggattatctggatgtcta

SEQ ID NO: 88 - RNA, 18 nt, Homo sapiens
  source 1..18: mol_type = genomic RNA
acgttgccctgatcaagc

SEQ ID NO: 89 - RNA, 18 nt, Homo sapiens
  source 1..18: mol_type = genomic RNA
ggagccaaaaagtgtcta

SEQ ID NO: 90 - RNA, 18 nt, Homo sapiens
  source 1..18: mol_type = genomic RNA
ccgattaccacaagcaac

SEQ ID NO: 91 - RNA, 18 nt, Homo sapiens
  source 1..18: mol_type = genomic RNA
aatgcagactgggtcacg

SEQ ID NO: 92 - RNA, 18 nt, Homo sapiens
  source 1..18: mol_type = genomic RNA
ctcacgcccgagactttc

SEQ ID NO: 93 - RNA, 18 nt, Homo sapiens
  source 1..18: mol_type = genomic RNA
cagcgatctgtgacaacg

SEQ ID NO: 94 - RNA, 18 nt, Homo sapiens
  source 1..18: mol_type = genomic RNA
ctaccacttgccagcaac

SEQ ID NO: 95 - RNA, 18 nt, Homo sapiens
  source 1..18: mol_type = genomic RNA
tttatgactatgacgttg

SEQ ID NO: 96 - RNA, 18 nt, Homo sapiens
  source 1..18: mol_type = genomic RNA
ccttgatagttcacaaga

SEQ ID NO: 97 - RNA, 18 nt, Homo sapiens
  source 1..18: mol_type = genomic RNA
ctcaagaggtggccgaag

SEQ ID NO: 98 - RNA, 18 nt, Homo sapiens
  source 1..18: mol_type = genomic RNA
ttttatgactatgacgtt

SEQ ID NO: 99 - RNA, 18 nt, Homo sapiens
  source 1..18: mol_type = genomic RNA
ttccgacttctccaagag

SEQ ID NO: 100 - RNA, 18 nt, synthetic construct
  source 1..18: mol_type = other RNA
cactcacattgtagtagg

SEQ ID NO: 101 - RNA, 18 nt, synthetic construct
  source 1..18: mol_type = other RNA
acgggtagaagccagaag

SEQ ID NO: 102 - RNA, 18 nt, synthetic construct
  source 1..18: mol_type = other RNA
tacgggtagaagccagaa

SEQ ID NO: 103 - RNA, 18 nt, synthetic construct
  source 1..18: mol_type = other RNA
gtgtaaccgtcatagcag

SEQ ID NO: 104 - RNA, 18 nt, synthetic construct
  source 1..18: mol_type = other RNA
agacatccactactcccc

SEQ ID NO: 105 - RNA, 18 nt, synthetic construct
  source 1..18: mol_type = other RNA
aagtactcagacaccaca

SEQ ID NO: 106 - RNA, 18 nt, synthetic construct
  source 1..18: mol_type = other RNA
gccagaaggacacacgta

SEQ ID NO: 107 - RNA, 18 nt, synthetic construct
  source 1..18: mol_type = other RNA
taagttgactagacactt

SEQ ID NO: 108 - RNA, 18 nt, synthetic construct
  source 1..18: mol_type = other RNA
atcagggcaacgtcatag

SEQ ID NO: 109 - RNA, 18 nt, synthetic construct
  source 1..18: mol_type = other RNA
cagtacaaaggaaccgag

SEQ ID NO: 110 - RNA, 18 nt, synthetic construct
  source 1..18: mol_type = other RNA
atcgactccttctatggt

SEQ ID NO: 111 - RNA, 18 nt, synthetic construct
  source 1..18: mol_type = other RNA
tagacatccagataatcc

SEQ ID NO: 112 - RNA, 18 nt, synthetic construct
  source 1..18: mol_type = other RNA
gcttgatcagggcaacgt

SEQ ID NO: 113 - RNA, 18 nt, synthetic construct
  source 1..18: mol_type = other RNA
tagacactttttggctcc

SEQ ID NO: 114 - RNA, 18 nt, synthetic construct
  source 1..18: mol_type = other RNA
gttgcttgtggtaatcgg

SEQ ID NO: 115 - RNA, 18 nt, synthetic construct
  source 1..18: mol_type = other RNA
cgtgacccagtctgcatt

SEQ ID NO: 116 - RNA, 18 nt, synthetic construct
  source 1..18: mol_type = other RNA
gaaagtctcgggcgtgag

SEQ ID NO: 117 - RNA, 18 nt, synthetic construct
  source 1..18: mol_type = other RNA
cgttgtcacagatcgctg

SEQ ID NO: 118 - RNA, 18 nt, synthetic construct
  source 1..18: mol_type = other RNA
gttgctggcaagtggtag

SEQ ID NO: 119 - RNA, 18 nt, synthetic construct
  source 1..18: mol_type = other RNA
caacgtcatagtcataaa

SEQ ID NO: 120 - RNA, 18 nt, synthetic construct
  source 1..18: mol_type = other RNA
tcttgtgaactatcaagg

SEQ ID NO: 121 - RNA, 18 nt, synthetic construct
  source 1..18: mol_type = other RNA
cttcggccacctcttgag

SEQ ID NO: 122 - RNA, 18 nt, synthetic construct
  source 1..18: mol_type = other RNA
aacgtcatagtcataaaa

SEQ ID NO: 123 - RNA, 18 nt, synthetic construct
  source 1..18: mol_type = other RNA
ctcttggagaagtcggaa

SEQ ID NO: 124 - RNA, 19 nt, synthetic construct
  source 1..19: mol_type = other RNA
cctactacaatgtgagtga

SEQ ID NO: 125 - RNA, 19 nt, synthetic construct
  source 1..19: mol_type = other RNA
cttctggcttctacccgta

SEQ ID NO: 126 - RNA, 19 nt, synthetic construct
  source 1..19: mol_type = other RNA
ttctggcttctacccgtaa

SEQ ID NO: 127 - RNA, 19 nt, synthetic construct
  source 1..19: mol_type = other RNA
ctgctatgacggttacaca

SEQ ID NO: 128 - RNA, 19 nt, synthetic construct
  source 1..19: mol_type = other RNA
ggggagtagtggatgtcta

SEQ ID NO: 129 - RNA, 19 nt, synthetic construct
  source 1..19: mol_type = other RNA
tgtggtgtctgagtactta

SEQ ID NO: 130 - RNA, 19 nt, synthetic construct
  source 1..19: mol_type = other RNA
tacgtgtgtccttctggca

SEQ ID NO: 131 - RNA, 19 nt, synthetic construct
  source 1..19: mol_type = other RNA
aagtgtctagtcaacttaa

SEQ ID NO: 132 - RNA, 19 nt, synthetic construct
  source 1..19: mol_type = other RNA
ctatgacgttgccctgata

SEQ ID NO: 133 - RNA, 19 nt, synthetic construct
  source 1..19: mol_type = other RNA
ctcggttcctttgtactga

SEQ ID NO: 134 - RNA, 19 nt, synthetic construct
  source 1..19: mol_type = other RNA
accatagaaggagtcgata

SEQ ID NO: 135 - RNA, 19 nt, synthetic construct
  source 1..19: mol_type = other RNA
ggattatctggatgtctaa

SEQ ID NO: 136 - RNA, 19 nt, synthetic construct
  source 1..19: mol_type = other RNA
acgttgccctgatcaagca

SEQ ID NO: 137 - RNA, 19 nt, synthetic construct
  source 1..19: mol_type = other RNA
ggagccaaaaagtgtctaa

SEQ ID NO: 138 - RNA, 19 nt, synthetic construct
  source 1..19: mol_type = other RNA
ccgattaccacaagcaaca

SEQ ID NO: 139 - RNA, 19 nt, synthetic construct
  source 1..19: mol_type = other RNA
aatgcagactgggtcacga

SEQ ID NO: 140 - RNA, 19 nt, synthetic construct
  source 1..19: mol_type = other RNA
ctcacgcccgagactttca

SEQ ID NO: 141 - RNA, 19 nt, synthetic construct
  source 1..19: mol_type = other RNA
cagcgatctgtgacaacga

SEQ ID NO: 142 - RNA, 19 nt, synthetic construct
  source 1..19: mol_type = other RNA
ctaccacttgccagcaaca

SEQ ID NO: 143 - RNA, 19 nt, synthetic construct
  source 1..19: mol_type = other RNA
tttatgactatgacgttga

SEQ ID NO: 144 - RNA, 19 nt, synthetic construct
  source 1..19: mol_type = other RNA
ccttgatagttcacaagaa

SEQ ID NO: 145 - RNA, 19 nt, synthetic construct
  source 1..19: mol_type = other RNA
ctcaagaggtggccgaaga

SEQ ID NO: 146 - RNA, 19 nt, synthetic construct
  source 1..19: mol_type = other RNA
ttttatgactatgacgtta

SEQ ID NO: 147 - RNA, 19 nt, synthetic construct
  source 1..19: mol_type = other RNA
ttccgacttctccaagaga

SEQ ID NO: 148 - RNA, 21 nt, synthetic construct
  source 1..21: mol_type = other RNA
tcactcacattgtagtaggga

SEQ ID NO: 149 - RNA, 21 nt, synthetic construct
  source 1..21: mol_type = other RNA
tacgggtagaagccagaagga

SEQ ID NO: 150 - RNA, 21 nt, synthetic construct
  source 1..21: mol_type = other RNA
ttacgggtagaagccagaagg

SEQ ID NO: 151 - RNA, 21 nt, synthetic construct
  source 1..21: mol_type = other RNA
tgtgtaaccgtcatagcagtg

SEQ ID NO: 152 - RNA, 21 nt, synthetic construct
  source 1..21: mol_type = other RNA
tagacatccactactccccag

SEQ ID NO: 153 - RNA, 21 nt, synthetic construct
  source 1..21: mol_type = other RNA
taagtactcagacaccacagc

SEQ ID NO: 154 - RNA, 21 nt, synthetic construct
  source 1..21: mol_type = other RNA
tgccagaaggacacacgtact

SEQ ID NO: 155 - RNA, 21 nt, synthetic construct
  source 1..21: mol_type = other RNA
ttaagttgactagacactttt

SEQ ID NO: 156 - RNA, 21 nt, synthetic construct
  source 1..21: mol_type = other RNA
tatcagggcaacgtcatagtc

SEQ ID NO: 157 - RNA, 21 nt, synthetic construct
  source 1..21: mol_type = other RNA
tcagtacaaaggaaccgaggg

SEQ ID NO: 158 - RNA, 21 nt, synthetic construct
  source 1..21: mol_type = other RNA
tatcgactccttctatggtct

SEQ ID NO: 159 - RNA, 21 nt, synthetic construct
  source 1..21: mol_type = other RNA
ttagacatccagataatcctc

SEQ ID NO: 160 - RNA, 21 nt, synthetic construct
  source 1..21: mol_type = other RNA
tgcttgatcagggcaacgtca

SEQ ID NO: 161 - RNA, 21 nt, synthetic construct
  source 1..21: mol_type = other RNA
ttagacactttttggctcctg

SEQ ID NO: 162 - RNA, 21 nt, synthetic construct
  source 1..21: mol_type = other RNA
tgttgcttgtggtaatcggta

SEQ ID NO: 163 - RNA, 21 nt, synthetic construct
  source 1..21: mol_type = other RNA
tcgtgacccagtctgcattac

SEQ ID NO: 164 - RNA, 21 nt, synthetic construct
  source 1..21: mol_type = other RNA
tgaaagtctcgggcgtgagca

SEQ ID NO: 165 - RNA, 21 nt, synthetic construct
  source 1..21: mol_type = other RNA
tcgttgtcacagatcgctgtc

SEQ ID NO: 166 - RNA, 21 nt, synthetic construct
  source 1..21: mol_type = other RNA
tgttgctggcaagtggtagtt

SEQ ID NO: 167 - RNA, 21 nt, synthetic construct
  source 1..21: mol_type = other RNA
tcaacgtcatagtcataaaat

SEQ ID NO: 168 - RNA, 21 nt, synthetic construct
  source 1..21: mol_type = other RNA
ttcttgtgaactatcaagggg

SEQ ID NO: 169 - RNA, 21 nt, synthetic construct
  source 1..21: mol_type = other RNA
tcttcggccacctcttgaggg

SEQ ID NO: 170 - RNA, 21 nt, synthetic construct
  source 1..21: mol_type = other RNA
taacgtcatagtcataaaatt

SEQ ID NO: 171 - RNA, 21 nt, synthetic construct
  source 1..21: mol_type = other RNA
tctcttggagaagtcggaagg

SEQ ID NO: 172 - RNA, 20 nt, synthetic construct
  source 1..20: mol_type = other RNA
cactcacattgtagtaggtt

SEQ ID NO: 173 - RNA, 20 nt, synthetic construct
  source 1..20: mol_type = other RNA
acgggtagaagccagaagtt

SEQ ID NO: 174 - RNA, 20 nt, synthetic construct
  source 1..20: mol_type = other RNA
tacgggtagaagccagaatt

SEQ ID NO: 175 - RNA, 20 nt, synthetic construct
  source 1..20: mol_type = other RNA
gtgtaaccgtcatagcagtt

SEQ ID NO: 176 - RNA, 20 nt, synthetic construct
  source 1..20: mol_type = other RNA
agacatccactactcccctt

SEQ ID NO: 177 - RNA, 20 nt, synthetic construct
  source 1..20: mol_type = other RNA
aagtactcagacaccacatt

SEQ ID NO: 178 - RNA, 20 nt, synthetic construct
  source 1..20: mol_type = other RNA
gccagaaggacacacgtatt

SEQ ID NO: 179 - RNA, 20 nt, synthetic construct
  source 1..20: mol_type = other RNA
taagttgactagacactttt

SEQ ID NO: 180 - RNA, 20 nt, synthetic construct
  source 1..20: mol_type = other RNA
atcagggcaacgtcatagtt

SEQ ID NO: 181 - RNA, 20 nt, synthetic construct
  source 1..20: mol_type = other RNA
cagtacaaaggaaccgagtt

SEQ ID NO: 182 - RNA, 20 nt, synthetic construct
  source 1..20: mol_type = other RNA
atcgactccttctatggttt

SEQ ID NO: 183 - RNA, 20 nt, synthetic construct
  source 1..20: mol_type = other RNA
tagacatccagataatcctt

SEQ ID NO: 184 - RNA, 20 nt, synthetic construct
  source 1..20: mol_type = other RNA
gcttgatcagggcaacgttt

SEQ ID NO: 185 - RNA, 20 nt, synthetic construct
  source 1..20: mol_type = other RNA
tagacactttttggctcctt

SEQ ID NO: 186 - RNA, 20 nt, synthetic construct
  source 1..20: mol_type = other RNA
gttgcttgtggtaatcggtt

SEQ ID NO: 187 - RNA, 20 nt, synthetic construct
  source 1..20: mol_type = other RNA
cgtgacccagtctgcatttt

SEQ ID NO: 188 - RNA, 20 nt, synthetic construct
  source 1..20: mol_type = other RNA
gaaagtctcgggcgtgagtt

SEQ ID NO: 189 - RNA, 20 nt, synthetic construct
  source 1..20: mol_type = other RNA
cgttgtcacagatcgctgtt

SEQ ID NO: 190 - RNA, 20 nt, synthetic construct
  source 1..20: mol_type = other RNA
gttgctggcaagtggtagtt

SEQ ID NO: 191 - RNA, 20 nt, synthetic construct
  source 1..20: mol_type = other RNA
caacgtcatagtcataaatt

SEQ ID NO: 192 - RNA, 20 nt, synthetic construct
  source 1..20: mol_type = other RNA
tcttgtgaactatcaaggtt

SEQ ID NO: 193 - RNA, 20 nt, synthetic construct
  source 1..20: mol_type = other RNA
cttcggccacctcttgagtt

SEQ ID NO: 194 - RNA, 20 nt, synthetic construct
  source 1..20: mol_type = other RNA
aacgtcatagtcataaaatt

SEQ ID NO: 195 - RNA, 20 nt, synthetic construct
  source 1..20: mol_type = other RNA
ctcttggagaagtcggaatt

SEQ ID NO: 196 - RNA, 21 nt, synthetic construct
  source 1..21: mol_type = other RNA
tcactcacattgtagtaggtt

SEQ ID NO: 197 - RNA, 21 nt, synthetic construct
  source 1..21: mol_type = other RNA
tacgggtagaagccagaagtt

SEQ ID NO: 198 - RNA, 21 nt, synthetic construct
  source 1..21: mol_type = other RNA
ttacgggtagaagccagaatt

SEQ ID NO: 199 - RNA, 21 nt, synthetic construct
  source 1..21: mol_type = other RNA
tgtgtaaccgtcatagcagtt

SEQ ID NO: 200 - RNA, 21 nt, synthetic construct
  source 1..21: mol_type = other RNA
tagacatccactactcccctt

SEQ ID NO: 201 - RNA, 21 nt, synthetic construct
  source 1..21: mol_type = other RNA
taagtactcagacaccacatt

SEQ ID NO: 202 - RNA, 21 nt, synthetic construct
  source 1..21: mol_type = other RNA
tgccagaaggacacacgtatt

SEQ ID NO: 203 - RNA, 21 nt, synthetic construct
  source 1..21: mol_type = other RNA
ttaagttgactagacactttt

SEQ ID NO: 204 - RNA, 21 nt, synthetic construct
  source 1..21: mol_type = other RNA
tatcagggcaacgtcatagtt

SEQ ID NO: 205 - RNA, 21 nt, synthetic construct
  source 1..21: mol_type = other RNA
tcagtacaaaggaaccgagtt

SEQ ID NO: 206 - RNA, 21 nt, synthetic construct
  source 1..21: mol_type = other RNA
tatcgactccttctatggttt

SEQ ID NO: 207 - RNA, 21 nt, synthetic construct
  source 1..21: mol_type = other RNA
ttagacatccagataatcctt

SEQ ID NO: 208 - RNA, 21 nt, synthetic construct
  source 1..21: mol_type = other RNA
tgcttgatcagggcaacgttt

SEQ ID NO: 209 - RNA, 21 nt, synthetic construct
  source 1..21: mol_type = other RNA
ttagacactttttggctcctt

SEQ ID NO: 210 - RNA, 21 nt, synthetic construct
  source 1..21: mol_type = other RNA
tgttgcttgtggtaatcggtt

SEQ ID NO: 211 - RNA, 21 nt, synthetic construct
  source 1..21: mol_type = other RNA
tcgtgacccagtctgcatttt

SEQ ID NO: 212 - RNA, 21 nt, synthetic construct
  source 1..21: mol_type = other RNA
tgaaagtctcgggcgtgagtt

SEQ ID NO: 213 - RNA, 21 nt, synthetic construct
  source 1..21: mol_type = other RNA
tcgttgtcacagatcgctgtt

SEQ ID NO: 214 - RNA, 21 nt, synthetic construct
  source 1..21: mol_type = other RNA
tgttgctggcaagtggtagtt

SEQ ID NO: 215 - RNA, 21 nt, synthetic construct
  source 1..21: mol_type = other RNA
tcaacgtcatagtcataaatt

SEQ ID NO: 216 - RNA, 21 nt, synthetic construct
  source 1..21: mol_type = other RNA
ttcttgtgaactatcaaggtt

SEQ ID NO: 217 - RNA, 21 nt, synthetic construct
  source 1..21: mol_type = other RNA
tcttcggccacctcttgagtt

SEQ ID NO: 218 - RNA, 21 nt, synthetic construct
  source 1..21: mol_type = other RNA
taacgtcatagtcataaaatt

SEQ ID NO: 219 - RNA, 21 nt, synthetic construct
  source 1..21: mol_type = other RNA
tctcttggagaagtcggaatt

SEQ ID NO: 220 - RNA, 19 nt, synthetic construct
  source 1..19: mol_type = other RNA
  modified_base 1: mod_base = OTHER; note = 2'-O-methyl nucleotide phosphorothioate
  modified_base 2: mod_base = OTHER; note = 2'-O-methyl nucleotide phosphorothioate
  modified_base 3: mod_base = OTHER; note = 2'-Fluoro nucleotide
  modified_base 4: mod_base = OTHER; note = 2'-O-methyl nucleotide
  modified_base 5: mod_base = OTHER; note = 2'-O-methyl nucleotide
  modified_base 6: mod_base = OTHER; note = 2'-O-methyl nucleotide
  modified_base 7: mod_base = OTHER; note = 2'-Fluoro nucleotide
  modified_base 8: mod_base = OTHER; note = 2'-Fluoro nucleotide
  modified_base 9: mod_base = OTHER; note = 2'-Fluoro nucleotide
  modified_base 10: mod_base = OTHER; note = 2'-O-methyl nucleotide
  modified_base 11: mod_base = OTHER; note = 2'-O-methyl nucleotide
  modified_base 12: mod_base = OTHER; note = 2'-Fluoro nucleotide
  modified_base 13: mod_base = OTHER; note = 2'-O-methyl nucleotide
  modified_base 14: mod_base = OTHER; note = 2'-O-methyl nucleotide
  modified_base 15: mod_base = OTHER; note = 2'-O-methyl nucleotide
  modified_base 16: mod_base = OTHER; note = 2'-O-methyl nucleotide
  modified_base 17: mod_base = OTHER; note = 2'-Fluoro nucleotide
  modified_base 18: mod_base = OTHER; note = 2'-O-methyl nucleotide
  modified_base 19: mod_base = OTHER; note = 2'-O-methyl nucleotide
cctactacaatgtgagtga

SEQ ID NO: 221 - RNA, 21 nt, synthetic construct
  source 1..21: mol_type = other RNA
  modified_base 1: mod_base = OTHER; note = 2'-O-methyl nucleotide phosphorothioate
  modified_base 2: mod_base = OTHER; note = 2'-Fluoro nucleotide phosphorothioate
  modified_base 3: mod_base = OTHER; note = 2'-O-methyl nucleotide
  modified_base 4: mod_base = OTHER; note = 2'-O-methyl nucleotide
  modified_base 5: mod_base = OTHER; note = 2'-O-methyl nucleotide
  modified_base 6: mod_base = OTHER; note = 2'-O-methyl nucleotide
  modified_base 7: mod_base = OTHER; note = 2'-O-methyl nucleotide
  modified_base 8: mod_base = OTHER; note = 2'-O-methyl nucleotide
  modified_base 9: mod_base = OTHER; note = 2'-O-methyl nucleotide
  modified_base 10: mod_base = OTHER; note = 2'-O-methyl nucleotide
  modified_base 11: mod_base = OTHER; note = 2'-O-methyl nucleotide
  modified_base 12: mod_base = OTHER; note = 2'-O-methyl nucleotide
  modified_base 13: mod_base = OTHER; note = 2'-O-methyl nucleotide
  modified_base 14: mod_base = OTHER; note = 2'-Fluoro nucleotide
  modified_base 15: mod_base = OTHER; note = 2'-O-methyl nucleotide
  modified_base 16: mod_base = OTHER; note = 2'-O-methyl nucleotide
  modified_base 17: mod_base = OTHER; note = 2'-O-methyl nucleotide
  modified_base 18: mod_base = OTHER; note = 2'-O-methyl nucleotide
  modified_base 19: mod_base = OTHER; note = 2'-O-methyl nucleotide phosphorothioate
  modified_base 20: mod_base = OTHER; note = 2'-O-methyl nucleotide phosphorothioate
  modified_base 21: mod_base = OTHER; note = 2'-O-methyl nucleotide
tcactcacattgtagtaggtt

SEQ ID NO: 222 - RNA, 19 nt, synthetic construct
  source 1..19: mol_type = other RNA
  modified_base 1: mod_base = OTHER; note = 2'-O-methyl nucleotide phosphorothioate
  modified_base 2: mod_base = OTHER; note = 2'-O-methyl nucleotide phosphorothioate
  modified_base 3: mod_base = OTHER; note = 2'-Fluoro nucleotide
  modified_base 4: mod_base = OTHER; note = 2'-O-methyl nucleotide
  modified_base 5: mod_base = OTHER; note = 2'-O-methyl nucleotide
  modified_base 6: mod_base = OTHER; note = 2'-O-methyl nucleotide
  modified_base 7: mod_base = OTHER; note = 2'-Fluoro nucleotide
  modified_base 8: mod_base = OTHER; note = 2'-Fluoro nucleotide
  modified_base 9: mod_base = OTHER; note = 2'-Fluoro nucleotide
  modified_base 10: mod_base = OTHER; note = 2'-O-methyl nucleotide
  modified_base 11: mod_base = OTHER; note = 2'-O-methyl nucleotide
  modified_base 12: mod_base = OTHER; note = 2'-Fluoro nucleotide
  modified_base 13: mod_base = OTHER; note = 2'-O-methyl nucleotide
  modified_base 14: mod_base = OTHER; note = 2'-O-methyl nucleotide
  modified_base 15: mod_base = OTHER; note = 2'-O-methyl nucleotide
  modified_base 16: mod_base = OTHER; note = 2'-O-methyl nucleotide
  modified_base 17: mod_base = OTHER; note = 2'-Fluoro nucleotide
  modified_base 18: mod_base = OTHER; note = 2'-O-methyl nucleotide
  modified_base 19: mod_base = OTHER; note = 2'-O-methyl nucleotide
cttctggcttctacccgta

SEQ ID NO: 223 - RNA, 21 nt, synthetic construct
  source 1..21: mol_type = other RNA
  modified_base 1: mod_base = OTHER; note = 2'-O-methyl nucleotide phosphorothioate
  modified_base 2: mod_base = OTHER; note = 2'-Fluoro nucleotide phosphorothioate
  modified_base 3: mod_base = OTHER; note = 2'-O-methyl nucleotide
  modified_base 4: mod_base = OTHER; note = 2'-O-methyl nucleotide
  modified_base 5: mod_base = OTHER; note = 2'-O-methyl nucleotide
  modified_base 6: mod_base = OTHER; note = 2'-O-methyl nucleotide
  modified_base 7: mod_base = OTHER; note = 2'-O-methyl nucleotide
  modified_base 8: mod_base = OTHER; note = 2'-O-methyl nucleotide
  modified_base 9: mod_base = OTHER; note = 2'-O-methyl nucleotide
  modified_base 10: mod_base = OTHER; note = 2'-O-methyl nucleotide
  modified_base 11: mod_base = OTHER; note = 2'-O-methyl nucleotide
  modified_base 12: mod_base = OTHER; note = 2'-O-methyl nucleotide
  modified_base 13: mod_base = OTHER; note = 2'-O-methyl nucleotide
  modified_base 14: mod_base = OTHER; note = 2'-Fluoro nucleotide
  modified_base 15: mod_base = OTHER; note = 2'-O-methyl nucleotide
  modified_base 16: mod_base = OTHER; note = 2'-O-methyl nucleotide
  modified_base 17: mod_base = OTHER; note = 2'-O-methyl nucleotide
  modified_base 18: mod_base = OTHER; note = 2'-O-methyl nucleotide
  modified_base 19: mod_base = OTHER; note = 2'-O-methyl nucleotide phosphorothioate
  modified_base 20: mod_base = OTHER; note = 2'-O-methyl nucleotide phosphorothioate
  modified_base 21: mod_base = OTHER; note = 2'-O-methyl nucleotide
tacgggtagaagccagaagtt

SEQ ID NO: 224 - RNA, 19 nt, synthetic construct
  source 1..19: mol_type = other RNA
  modified_base 1: mod_base = OTHER; note = 2'-O-methyl nucleotide phosphorothioate
  modified_base 2: mod_base = OTHER; note = 2'-O-methyl nucleotide phosphorothioate
  modified_base 3: mod_base = OTHER; note = 2'-Fluoro nucleotide
  modified_base 4: mod_base = OTHER; note = 2'-O-methyl nucleotide
  modified_base 5: mod_base = OTHER; note = 2'-O-methyl nucleotide
  modified_base 6: mod_base = OTHER; note = 2'-O-methyl nucleotide
  modified_base 7: mod_base = OTHER; note = 2'-Fluoro nucleotide
  modified_base 8: mod_base = OTHER; note = 2'-Fluoro nucleotide
  modified_base 9: mod_base = OTHER; note = 2'-Fluoro nucleotide
  modified_base 10: mod_base = OTHER; note = 2'-O-methyl nucleotide
  modified_base 11: mod_base = OTHER; note = 2'-O-methyl nucleotide
  modified_base 12: mod_base = OTHER; note = 2'-Fluoro nucleotide
  modified_base 13: mod_base = OTHER; note = 2'-O-methyl nucleotide
  modified_base 14: mod_base = OTHER; note = 2'-O-methyl nucleotide
  modified_base 15: mod_base = OTHER; note = 2'-O-methyl nucleotide
  modified_base 16: mod_base = OTHER; note = 2'-O-methyl nucleotide
  modified_base 17: mod_base = OTHER; note = 2'-Fluoro nucleotide
  modified_base 18: mod_base = OTHER; note = 2'-O-methyl nucleotide
  modified_base 19: mod_base = OTHER; note = 2'-O-methyl nucleotide
ttctggcttctacccgtaa

SEQ ID NO: 225 - RNA, 21 nt, synthetic construct
  source 1..21: mol_type = other RNA
  modified_base 1: mod_base = OTHER; note = 2'-O-methyl nucleotide phosphorothioate
  modified_base 2: mod_base = OTHER; note = 2'-Fluoro nucleotide phosphorothioate
  modified_base 3: mod_base = OTHER; note = 2'-O-methyl nucleotide
  modified_base 4: mod_base = OTHER; note = 2'-O-methyl nucleotide
  modified_base 5: mod_base = OTHER; note = 2'-O-methyl nucleotide
  modified_base 6: mod_base = OTHER; note = 2'-O-methyl nucleotide
  modified_base 7: mod_base = OTHER; note = 2'-O-methyl nucleotide
  modified_base 8: mod_base = OTHER; note = 2'-O-methyl nucleotide
  modified_base 9: mod_base = OTHER; note = 2'-O-methyl nucleotide
  modified_base 10: mod_base = OTHER; note = 2'-O-methyl nucleotide
  modified_base 11: mod_base = OTHER; note = 2'-O-methyl nucleotide
  modified_base 12: mod_base = OTHER; note = 2'-O-methyl nucleotide
  modified_base 13: mod_base = OTHER; note = 2'-O-methyl nucleotide
  modified_base 14: mod_base = OTHER; note = 2'-Fluoro nucleotide
  modified_base 15: mod_base = OTHER; note = 2'-O-methyl nucleotide
  modified_base 16: mod_base = OTHER; note = 2'-O-methyl nucleotide
  modified_base 17: mod_base = OTHER; note = 2'-O-methyl nucleotide
  modified_base 18: mod_base = OTHER; note = 2'-O-methyl nucleotide
  modified_base 19: mod_base = OTHER; note = 2'-O-methyl nucleotide phosphorothioate
  modified_base 20: mod_base = OTHER; note = 2'-O-methyl nucleotide phosphorothioate
  modified_base 21: mod_base = OTHER; note = 2'-O-methyl nucleotide
ttacgggtagaagccagaatt

SEQ ID NO: 226 - RNA, 19 nt, synthetic construct
  source 1..19: mol_type = other RNA
  modified_base 1: mod_base = OTHER; note = 2'-O-methyl nucleotide phosphorothioate
  modified_base 2: mod_base = OTHER; note = 2'-O-methyl nucleotide phosphorothioate
  modified_base 3: mod_base = OTHER; note = 2'-Fluoro nucleotide
  modified_base 4: mod_base = OTHER; note = 2'-O-methyl nucleotide
  modified_base 5: mod_base = OTHER; note = 2'-O-methyl nucleotide
  modified_base 6: mod_base = OTHER; note = 2'-O-methyl nucleotide
  modified_base 7: mod_base = OTHER; note = 2'-Fluoro nucleotide
  modified_base 8: mod_base = OTHER; note = 2'-Fluoro nucleotide
  modified_base 9: mod_base = OTHER; note = 2'-Fluoro nucleotide
  modified_base 10: mod_base = OTHER; note = 2'-O-methyl nucleotide
  modified_base 11: mod_base = OTHER; note = 2'-O-methyl nucleotide
  modified_base 12: mod_base = OTHER; note = 2'-Fluoro nucleotide
  modified_base 13: mod_base = OTHER; note = 2'-O-methyl nucleotide
  modified_base 14: mod_base = OTHER; note = 2'-O-methyl nucleotide
  modified_base 15: mod_base = OTHER; note = 2'-O-methyl nucleotide
  modified_base 16: mod_base = OTHER; note = 2'-O-methyl nucleotide
  modified_base 17: mod_base = OTHER; note = 2'-Fluoro nucleotide
  modified_base 18: mod_base = OTHER; note = 2'-O-methyl nucleotide
  modified_base 19: mod_base = OTHER; note = 2'-O-methyl nucleotide
ctgctatgacggttacaca

SEQ ID NO: 227 - RNA, 21 nt, synthetic construct
  source 1..21: mol_type = other RNA
  modified_base 1: mod_base = OTHER; note = 2'-O-methyl nucleotide phosphorothioate
  modified_base 2: mod_base = OTHER; note = 2'-Fluoro nucleotide phosphorothioate
  modified_base 3: mod_base = OTHER; note = 2'-O-methyl nucleotide
  modified_base 4: mod_base = OTHER; note = 2'-O-methyl nucleotide
  modified_base 5: mod_base = OTHER; note = 2'-O-methyl nucleotide
  modified_base 6: mod_base = OTHER; note = 2'-O-methyl nucleotide
  modified_base 7: mod_base = OTHER; note = 2'-O-methyl nucleotide
  modified_base 8: mod_base = OTHER; note = 2'-O-methyl nucleotide
  modified_base 9: mod_base = OTHER; note = 2'-O-methyl nucleotide
  modified_base 10: mod_base = OTHER; note = 2'-O-methyl nucleotide
  modified_base 11: mod_base = OTHER; note = 2'-O-methyl nucleotide
  modified_base 12: mod_base = OTHER; note = 2'-O-methyl nucleotide
  modified_base 13: mod_base = OTHER; note = 2'-O-methyl nucleotide
  modified_base 14: mod_base = OTHER; note = 2'-Fluoro nucleotide
  modified_base 15: mod_base = OTHER; note = 2'-O-methyl nucleotide
  modified_base 16: mod_base = OTHER; note = 2'-O-methyl nucleotide
  modified_base 17: mod_base = OTHER; note = 2'-O-methyl nucleotide
  modified_base 18: mod_base = OTHER; note = 2'-O-methyl nucleotide
  modified_base 19: mod_base = OTHER; note = 2'-O-methyl nucleotide phosphorothioate
  modified_base 20: mod_base = OTHER; note = 2'-O-methyl nucleotide phosphorothioate
  modified_base 21: mod_base = OTHER; note = 2'-O-methyl nucleotide
tgtgtaaccgtcatagcagtt

SEQ ID NO: 228 - RNA, 19 nt, synthetic construct
  source 1..19: mol_type = other RNA
  modified_base 1: mod_base = OTHER; note = 2'-O-methyl nucleotide phosphorothioate
  modified_base 2: mod_base = OTHER; note = 2'-O-methyl nucleotide phosphorothioate
  modified_base 3: mod_base = OTHER; note = 2'-Fluoro nucleotide
  modified_base 4: mod_base = OTHER; note = 2'-O-methyl nucleotide
  modified_base 5: mod_base = OTHER; note = 2'-O-methyl nucleotide
  modified_base 6: mod_base = OTHER; note = 2'-O-methyl nucleotide
  modified_base 7: mod_base = OTHER; note = 2'-Fluoro nucleotide
  modified_base 8: mod_base = OTHER; note = 2'-Fluoro nucleotide
  modified_base 9: mod_base = OTHER; note = 2'-Fluoro nucleotide
  modified_base 10: mod_base = OTHER; note = 2'-O-methyl nucleotide
  modified_base 11: mod_base = OTHER; note = 2'-O-methyl nucleotide
  modified_base 12: mod_base = OTHER; note = 2'-Fluoro nucleotide
  modified_base 13: mod_base = OTHER; note = 2'-O-methyl nucleotide
  modified_base 14: mod_base = OTHER; note = 2'-O-methyl nucleotide
  modified_base 15: mod_base = OTHER; note = 2'-O-methyl nucleotide
  modified_base 16: mod_base = OTHER; note = 2'-O-methyl nucleotide
  modified_base 17: mod_base = OTHER; note = 2'-Fluoro nucleotide
  modified_base 18: mod_base = OTHER; note = 2'-O-methyl nucleotide
  modified_base 19: mod_base = OTHER; note = 2'-O-methyl nucleotide
ggggagtagtggatgtcta

SEQ ID NO: 229 - RNA, 21 nt, synthetic construct
  source 1..21: mol_type = other RNA
  modified_base 1: mod_base = OTHER; note = 2'-O-methyl nucleotide phosphorothioate
  modified_base 2: mod_base = OTHER; note = 2'-Fluoro nucleotide phosphorothioate
  modified_base 3: mod_base = OTHER; note = 2'-O-methyl nucleotide
  modified_base 4: mod_base = OTHER; note = 2'-O-methyl nucleotide
  modified_base 5: mod_base = OTHER; note = 2'-O-methyl nucleotide
  modified_base 6: mod_base = OTHER; note = 2'-O-methyl nucleotide
  modified_base 7: mod_base = OTHER; note = 2'-O-methyl nucleotide
  modified_base 8: mod_base = OTHER; note = 2'-O-methyl nucleotide
  modified_base 9: mod_base = OTHER; note = 2'-O-methyl nucleotide
  modified_base 10: mod_base = OTHER; note = 2'-O-methyl nucleotide
  modified_base 11: mod_base = OTHER; note = 2'-O-methyl nucleotide
  modified_base 12: mod_base = OTHER; note = 2'-O-methyl nucleotide
  modified_base 13: mod_base = OTHER; note = 2'-O-methyl nucleotide
  modified_base 14: mod_base = OTHER; note = 2'-Fluoro nucleotide
  modified_base 15: mod_base = OTHER; note = 2'-O-methyl nucleotide
  modified_base 16: mod_base = OTHER; note = 2'-O-methyl nucleotide
  modified_base 17: mod_base = OTHER; note = 2'-O-methyl nucleotide
  modified_base 18: mod_base = OTHER; note = 2'-O-methyl nucleotide
  modified_base 19: mod_base = OTHER; note = 2'-O-methyl nucleotide phosphorothioate
  modified_base 20: mod_base = OTHER; note = 2'-O-methyl nucleotide phosphorothioate
  modified_base 21: mod_base = OTHER; note = 2'-O-methyl nucleotide
tagacatccactactcccctt

SEQ ID NO: 230 - RNA, 19 nt, synthetic construct
  source 1..19: mol_type = other RNA
  modified_base 1: mod_base = OTHER; note = 2'-O-methyl nucleotide phosphorothioate
  modified_base 2: mod_base = OTHER; note = 2'-O-methyl nucleotide phosphorothioate
  modified_base 3: mod_base = OTHER; note = 2'-Fluoro nucleotide
  modified_base 4: mod_base = OTHER; note = 2'-O-methyl nucleotide
  modified_base 5: mod_base = OTHER; note = 2'-O-methyl nucleotide
  modified_base 6: mod_base = OTHER; note = 2'-O-methyl nucleotide
  modified_base 7: mod_base = OTHER; note = 2'-Fluoro nucleotide
  modified_base 8: mod_base = OTHER; note = 2'-Fluoro nucleotide
  modified_base 9: mod_base = OTHER; note = 2'-Fluoro nucleotide
  modified_base 10: mod_base = OTHER; note = 2'-O-methyl nucleotide
  modified_base 11: mod_base = OTHER; note = 2'-O-methyl nucleotide
  modified_base 12: mod_base = OTHER; note = 2'-Fluoro nucleotide
  modified_base 13: mod_base = OTHER; note = 2'-O-methyl nucleotide
  modified_base 14: mod_base = OTHER; note = 2'-O-methyl nucleotide
  modified_base 15: mod_base = OTHER; note = 2'-O-methyl nucleotide
  modified_base 16: mod_base = OTHER; note = 2'-O-methyl nucleotide
  modified_base 17: mod_base = OTHER; note = 2'-Fluoro nucleotide
  modified_base 18: mod_base = OTHER; note = 2'-O-methyl nucleotide
  modified_base 19: mod_base = OTHER; note = 2'-O-methyl nucleotide
tgtggtgtctgagtactta

SEQ ID NO: 231 - RNA, 21 nt, synthetic construct
  source 1..21: mol_type = other RNA
  modified_base 1: mod_base = OTHER; note = 2'-O-methyl nucleotide phosphorothioate
  modified_base 2: mod_base = OTHER; note = 2'-Fluoro nucleotide phosphorothioate
  modified_base 3: mod_base = OTHER; note = 2'-O-methyl nucleotide
  modified_base 4: mod_base = OTHER; note = 2'-O-methyl nucleotide
  modified_base 5: mod_base = OTHER; note = 2'-O-methyl nucleotide
  modified_base 6: mod_base = OTHER; note = 2'-O-methyl nucleotide
  modified_base 7: mod_base = OTHER; note = 2'-O-methyl nucleotide
  modified_base 8: mod_base = OTHER; note = 2'-O-methyl nucleotide
  modified_base 9: mod_base = OTHER; note = 2'-O-methyl nucleotide
  modified_base 10: mod_base = OTHER; note = 2'-O-methyl nucleotide
  modified_base 11: mod_base = OTHER; note = 2'-O-methyl nucleotide
  modified_base 12: mod_base = OTHER; note = 2'-O-methyl nucleotide
  modified_base 13: mod_base = OTHER; note = 2'-O-methyl nucleotide
  modified_base 14: mod_base = OTHER; note = 2'-Fluoro nucleotide
  modified_base 15: mod_base = OTHER; note = 2'-O-methyl nucleotide
  modified_base 16: mod_base = OTHER; note = 2'-O-methyl nucleotide
  modified_base 17: mod_base = OTHER; note = 2'-O-methyl nucleotide
  modified_base 18: mod_base = OTHER; note = 2'-O-methyl nucleotide
  modified_base 19: mod_base = OTHER; note = 2'-O-methyl nucleotide phosphorothioate
  modified_base 20: mod_base = OTHER; note = 2'-O-methyl nucleotide phosphorothioate
  modified_base 21: mod_base = OTHER; note = 2'-O-methyl nucleotide
taagtactcagacaccacatt

SEQ ID NO: 232 - RNA, 19 nt, synthetic construct
  source 1..19: mol_type = other RNA
  modified_base 1: mod_base = OTHER; note = 2'-O-methyl nucleotide phosphorothioate
  modified_base 2: mod_base = OTHER; note = 2'-O-methyl nucleotide phosphorothioate
  modified_base 3: mod_base = OTHER; note = 2'-Fluoro nucleotide
  modified_base 4: mod_base = OTHER; note = 2'-O-methyl nucleotide
  modified_base 5: mod_base = OTHER; note = 2'-O-methyl nucleotide
  modified_base 6: mod_base = OTHER; note = 2'-O-methyl nucleotide
  modified_base 7: mod_base = OTHER; note = 2'-Fluoro nucleotide
  modified_base 8: mod_base = OTHER; note = 2'-Fluoro nucleotide
  modified_base 9: mod_base = OTHER; note = 2'-Fluoro nucleotide
  modified_base 10: mod_base = OTHER; note = 2'-O-methyl nucleotide
  modified_base 11: mod_base = OTHER; note = 2'-O-methyl nucleotide
  modified_base 12: mod_base = OTHER; note = 2'-Fluoro nucleotide
  modified_base 13: mod_base = OTHER; note = 2'-O-methyl nucleotide
  modified_base 14: mod_base = OTHER; note = 2'-O-methyl nucleotide
  modified_base 15: mod_base = OTHER; note = 2'-O-methyl nucleotide
  modified_base 16: mod_base = OTHER; note = 2'-O-methyl nucleotide
  modified_base 17: mod_base = OTHER; note = 2'-Fluoro nucleotide
  modified_base 18: mod_base = OTHER; note = 2'-O-methyl nucleotide
  modified_base 19: mod_base = OTHER; note = 2'-O-methyl nucleotide
tacgtgtgtccttctggca

SEQ ID NO: 233 - RNA, 21 nt, synthetic construct
  source 1..21: mol_type = other RNA
  modified_base 1: mod_base = OTHER; note = 2'-O-methyl nucleotide phosphorothioate
  modified_base 2: mod_base = OTHER; note = 2'-Fluoro nucleotide phosphorothioate
  modified_base 3: mod_base = OTHER; note = 2'-O-methyl nucleotide
  modified_base 4: mod_base = OTHER; note = 2'-O-methyl nucleotide
  modified_base 5: mod_base = OTHER; note = 2'-O-methyl nucleotide
  modified_base 6: mod_base = OTHER; note = 2'-O-methyl nucleotide
  modified_base 7: mod_base = OTHER; note = 2'-O-methyl nucleotide
  modified_base 8: mod_base = OTHER; note = 2'-O-methyl nucleotide
  modified_base 9: mod_base = OTHER; note = 2'-O-methyl nucleotide
  modified_base 10: mod_base = OTHER; note = 2'-O-methyl nucleotide
  modified_base 11: mod_base = OTHER; note = 2'-O-methyl nucleotide
  modified_base 12: mod_base = OTHER; note = 2'-O-methyl nucleotide
  modified_base 13: mod_base = OTHER; note = 2'-O-methyl nucleotide
  modified_base 14: mod_base = OTHER; note = 2'-Fluoro nucleotide
  modified_base 15: mod_base = OTHER; note = 2'-O-methyl nucleotide
  modified_base 16: mod_base = OTHER; note = 2'-O-methyl nucleotide
  modified_base 17: mod_base = OTHER; note = 2'-O-methyl nucleotide
  modified_base 18: mod_base = OTHER; note = 2'-O-methyl nucleotide
  modified_base 19: mod_base = OTHER; note = 2'-O-methyl nucleotide phosphorothioate
  modified_base 20: mod_base = OTHER; note = 2'-O-methyl nucleotide phosphorothioate
  modified_base 21: mod_base = OTHER; note = 2'-O-methyl nucleotide
tgccagaaggacacacgtatt

SEQ ID NO: 234 - RNA, 19 nt, synthetic construct
  source 1..19: mol_type = other RNA
  modified_base 1: mod_base = OTHER; note = 2'-O-methyl nucleotide phosphorothioate
  modified_base 2: mod_base = OTHER; note = 2'-O-methyl nucleotide phosphorothioate
  modified_base 3: mod_base = OTHER; note = 2'-Fluoro nucleotide
  modified_base 4: mod_base = OTHER; note = 2'-O-methyl nucleotide
  modified_base 5: mod_base = OTHER; note = 2'-O-methyl nucleotide
  modified_base 6: mod_base = OTHER; note = 2'-O-methyl nucleotide
  modified_base 7: mod_base = OTHER; note = 2'-Fluoro nucleotide
  modified_base 8: mod_base = OTHER; note = 2'-Fluoro nucleotide
  modified_base 9: mod_base = OTHER; note = 2'-Fluoro nucleotide
  modified_base 10: mod_base = OTHER; note = 2'-O-methyl nucleotide
  modified_base 11: mod_base = OTHER; note = 2'-O-methyl nucleotide
  modified_base 12: mod_base = OTHER; note = 2'-Fluoro nucleotide
  modified_base 13: mod_base = OTHER; note = 2'-O-methyl nucleotide
  modified_base 14: mod_base = OTHER; note = 2'-O-methyl nucleotide
  modified_base 15: mod_base = OTHER; note = 2'-O-methyl nucleotide
  modified_base 16: mod_base = OTHER; note = 2'-O-methyl nucleotide
  modified_base 17: mod_base = OTHER; note = 2'-Fluoro nucleotide
  modified_base 18: mod_base = OTHER; note = 2'-O-methyl nucleotide
  modified_base 19: mod_base = OTHER; note = 2'-O-methyl nucleotide
aagtgtctagtcaacttaa

SEQ ID NO: 235 - RNA, 21 nt, synthetic construct
  source 1..21: mol_type = other RNA
  modified_base 1: mod_base = OTHER; note = 2'-O-methyl nucleotide phosphorothioate
  modified_base 2: mod_base = OTHER; note = 2'-Fluoro nucleotide phosphorothioate
  modified_base 3: mod_base = OTHER; note = 2'-O-methyl nucleotide
  modified_base 4: mod_base = OTHER; note = 2'-O-methyl nucleotide
  modified_base 5: mod_base = OTHER; note = 2'-O-methyl nucleotide
  modified_base 6: mod_base = OTHER; note = 2'-O-methyl nucleotide
  modified_base 7: mod_base = OTHER; note = 2'-O-methyl nucleotide
  modified_base 8: mod_base = OTHER; note = 2'-O-methyl nucleotide
  modified_base 9: mod_base = OTHER; note = 2'-O-methyl nucleotide
  modified_base 10: mod_base = OTHER; note = 2'-O-methyl nucleotide
  modified_base 11: mod_base = OTHER; note = 2'-O-methyl nucleotide
  modified_base 12: mod_base = OTHER; note = 2'-O-methyl nucleotide
  modified_base 13: mod_base = OTHER; note = 2'-O-methyl nucleotide
  modified_base 14: mod_base = OTHER; note = 2'-Fluoro nucleotide
  modified_base 15: mod_base = OTHER; note = 2'-O-methyl nucleotide
  modified_base 16: mod_base = OTHER; note = 2'-O-methyl nucleotide
  modified_base 17: mod_base = OTHER; note = 2'-O-methyl nucleotide
  modified_base 18: mod_base = OTHER; note = 2'-O-methyl nucleotide
  modified_base 19: mod_base = OTHER; note = 2'-O-methyl nucleotide phosphorothioate
  modified_base 20: mod_base = OTHER; note = 2'-O-methyl nucleotide phosphorothioate
  modified_base 21: mod_base = OTHER; note = 2'-O-methyl nucleotide
ttaagttgactagacactttt

SEQ ID NO: 236 - RNA, 19 nt, synthetic construct
  source 1..19: mol_type = other RNA
  modified_base 1: mod_base = OTHER; note = 2'-O-methyl nucleotide phosphorothioate
  modified_base 2: mod_base = OTHER; note = 2'-O-methyl nucleotide phosphorothioate
  modified_base 3: mod_base = OTHER; note = 2'-Fluoro nucleotide
  modified_base 4: mod_base = OTHER; note = 2'-O-methyl nucleotide
  modified_base 5: mod_base = OTHER; note = 2'-O-methyl nucleotide
  modified_base 6: mod_base = OTHER; note = 2'-O-methyl nucleotide
  modified_base 7: mod_base = OTHER; note = 2'-Fluoro nucleotide
  modified_base 8: mod_base = OTHER; note = 2'-Fluoro nucleotide
  modified_base 9: mod_base = OTHER; note = 2'-Fluoro nucleotide
  modified_base 10: mod_base = OTHER; note = 2'-O-methyl nucleotide
  modified_base 11: mod_base = OTHER; note = 2'-O-methyl nucleotide
  modified_base 12: mod_base = OTHER; note = 2'-Fluoro nucleotide
  modified_base 13: mod_base = OTHER; note = 2'-O-methyl nucleotide
  modified_base 14: mod_base = OTHER; note = 2'-O-methyl nucleotide
  modified_base 15: mod_base = OTHER; note = 2'-O-methyl nucleotide
  modified_base 16: mod_base = OTHER; note = 2'-O-methyl nucleotide
  modified_base 17: mod_base = OTHER; note = 2'-Fluoro nucleotide
  modified_base 18: mod_base = OTHER; note = 2'-O-methyl nucleotide
  modified_base 19: mod_base = OTHER; note = 2'-O-methyl nucleotide
ctatgacgttgccctgata

SEQ ID NO: 237 - RNA, 21 nt, synthetic construct
  source 1..21: mol_type = other RNA
  modified_base 1: mod_base = OTHER; note = 2'-O-methyl nucleotide phosphorothioate
  modified_base 2: mod_base = OTHER; note = 2'-Fluoro nucleotide phosphorothioate
  modified_base 3: mod_base = OTHER; note = 2'-O-methyl nucleotide
  modified_base 4: mod_base = OTHER; note = 2'-O-methyl nucleotide
  modified_base 5: mod_base = OTHER; note = 2'-O-methyl nucleotide
  modified_base 6: mod_base = OTHER; note = 2'-O-methyl nucleotide
  modified_base 7: mod_base = OTHER; note = 2'-O-methyl nucleotide
  modified_base 8: mod_base = OTHER; note = 2'-O-methyl nucleotide
  modified_base 9: mod_base = OTHER; note = 2'-O-methyl nucleotide
  modified_base 10: mod_base = OTHER; note = 2'-O-methyl nucleotide
  modified_base 11: mod_base = OTHER; note = 2'-O-methyl nucleotide
  modified_base 12: mod_base = OTHER; note = 2'-O-methyl nucleotide
  modified_base 13: mod_base = OTHER; note = 2'-O-methyl nucleotide
  modified_base 14: mod_base = OTHER; note = 2'-Fluoro nucleotide
  modified_base 15: mod_base = OTHER; note = 2'-O-methyl nucleotide
  modified_base 16: mod_base = OTHER; note = 2'-O-methyl nucleotide
  modified_base 17: mod_base = OTHER; note = 2'-O-methyl nucleotide
  modified_base 18: mod_base = OTHER; note = 2'-O-methyl nucleotide
  modified_base 19: mod_base = OTHER; note = 2'-O-methyl nucleotide phosphorothioate
  modified_base 20: mod_base = OTHER; note = 2'-O-methyl nucleotide phosphorothioate
  modified_base 21: mod_base = OTHER; note = 2'-O-methyl nucleotide
tatcagggcaacgtcatagtt

SEQ ID NO: 238 - RNA, 19 nt, synthetic construct
  source 1..19: mol_type = other RNA
  modified_base 1: mod_base = OTHER; note = 2'-O-methyl nucleotide phosphorothioate
  modified_base 2: mod_base = OTHER; note = 2'-O-methyl nucleotide phosphorothioate
  modified_base 3: mod_base = OTHER; note = 2'-Fluoro nucleotide
  modified_base 4: mod_base = OTHER; note = 2'-O-methyl nucleotide
  modified_base 5: mod_base = OTHER; note = 2'-O-methyl nucleotide
  modified_base 6: mod_base = OTHER; note = 2'-O-methyl nucleotide
  modified_base 7: mod_base = OTHER; note = 2'-Fluoro nucleotide
  modified_base 8: mod_base = OTHER; note = 2'-Fluoro nucleotide
  modified_base 9: mod_base = OTHER; note = 2'-Fluoro nucleotide
  modified_base 10: mod_base = OTHER; note = 2'-O-methyl nucleotide
  modified_base 11: mod_base = OTHER; note = 2'-O-methyl nucleotide
  modified_base 12: mod_base = OTHER; note = 2'-Fluoro nucleotide
  modified_base 13: mod_base = OTHER; note = 2'-O-methyl nucleotide
  modified_base 14: mod_base = OTHER; note = 2'-O-methyl nucleotide
  modified_base 15: mod_base = OTHER; note = 2'-O-methyl nucleotide
  modified_base 16: mod_base = OTHER; note = 2'-O-methyl nucleotide
  modified_base 17: mod_base = OTHER; note = 2'-Fluoro nucleotide
  modified_base 18: mod_base = OTHER; note = 2'-O-methyl nucleotide
  modified_base 19: mod_base = OTHER; note = 2'-O-methyl nucleotide
ctcggttcctttgtactga

SEQ ID NO: 239 - RNA, 21 nt, synthetic construct
  source 1..21: mol_type = other RNA
  modified_base 1: mod_base = OTHER; note = 2'-O-methyl nucleotide phosphorothioate
  modified_base 2: mod_base = OTHER; note = 2'-Fluoro nucleotide phosphorothioate
  modified_base 3: mod_base = OTHER; note = 2'-O-methyl nucleotide
  modified_base 4: mod_base = OTHER; note = 2'-O-methyl nucleotide
  modified_base 5: mod_base = OTHER; note = 2'-O-methyl nucleotide
  modified_base 6: mod_base = OTHER; note = 2'-O-methyl nucleotide
  modified_base 7: mod_base = OTHER; note = 2'-O-methyl nucleotide
  modified_base 8: mod_base = OTHER; note = 2'-O-methyl nucleotide
  modified_base 9: mod_base = OTHER; note = 2'-O-methyl nucleotide
  modified_base 10: mod_base = OTHER; note = 2'-O-methyl nucleotide
  modified_base 11: mod_base = OTHER; note = 2'-O-methyl nucleotide
  modified_base 12: mod_base = OTHER; note = 2'-O-methyl nucleotide
  modified_base 13: mod_base = OTHER; note = 2'-O-methyl nucleotide
  modified_base 14: mod_base = OTHER; note = 2'-Fluoro nucleotide
  modified_base 15: mod_base = OTHER; note = 2'-O-methyl nucleotide
  modified_base 16: mod_base = OTHER; note = 2'-O-methyl nucleotide
  modified_base 17: mod_base = OTHER; note = 2'-O-methyl nucleotide
  modified_base 18: mod_base = OTHER; note = 2'-O-methyl nucleotide
  modified_base 19: mod_base = OTHER; note = 2'-O-methyl nucleotide phosphorothioate
  modified_base 20: mod_base = OTHER; note = 2'-O-methyl nucleotide phosphorothioate
  modified_base 21: mod_base = OTHER; note = 2'-O-methyl nucleotide
tcagtacaaaggaaccgagtt

SEQ ID NO: 240 - RNA, 19 nt, synthetic construct
  source 1..19: mol_type = other RNA
  modified_base 1: mod_base = OTHER; note = 2'-O-methyl nucleotide phosphorothioate
  modified_base 2: mod_base = OTHER; note = 2'-O-methyl nucleotide phosphorothioate
  modified_base 3: mod_base = OTHER; note = 2'-Fluoro nucleotide
  modified_base 4: mod_base = OTHER; note = 2'-O-methyl nucleotide
  modified_base 5: mod_base = OTHER; note = 2'-O-methyl nucleotide
  modified_base 6: mod_base = OTHER; note = 2'-O-methyl nucleotide
  modified_base 7: mod_base = OTHER; note = 2'-Fluoro nucleotide
  modified_base 8: mod_base = OTHER; note = 2'-Fluoro nucleotide
  modified_base 9: mod_base = OTHER; note = 2'-Fluoro nucleotide
  modified_base 10: mod_base = OTHER; note = 2'-O-methyl nucleotide
  modified_base 11: mod_base = OTHER; note = 2'-O-methyl nucleotide
  modified_base 12: mod_base = OTHER; note = 2'-Fluoro nucleotide
  modified_base 13: mod_base = OTHER; note = 2'-O-methyl nucleotide
  modified_base 14: mod_base = OTHER; note = 2'-O-methyl nucleotide
  modified_base 15: mod_base = OTHER; note = 2'-O-methyl nucleotide
  modified_base 16: mod_base = OTHER; note = 2'-O-methyl nucleotide
  modified_base 17: mod_base = OTHER; note = 2'-Fluoro nucleotide
  modified_base 18: mod_base = OTHER; note = 2'-O-methyl nucleotide
  modified_base 19: mod_base = OTHER; note = 2'-O-methyl nucleotide
accatagaaggagtcgata

SEQ ID NO: 241 - RNA, 21 nt, synthetic construct
  source 1..21: mol_type = other RNA
  modified_base 1: mod_base = OTHER; note = 2'-O-methyl nucleotide phosphorothioate
  modified_base 2: mod_base = OTHER; note = 2'-Fluoro nucleotide phosphorothioate
  modified_base 3: mod_base = OTHER; note = 2'-O-methyl nucleotide
  modified_base 4: mod_base = OTHER; note = 2'-O-methyl nucleotide
  modified_base 5: mod_base = OTHER; note = 2'-O-methyl nucleotide
  modified_base 6: mod_base = OTHER; note = 2'-O-methyl nucleotide
  modified_base 7: mod_base = OTHER; note = 2'-O-methyl nucleotide
  modified_base 8: mod_base = OTHER; note = 2'-O-methyl nucleotide
  modified_base 9: mod_base = OTHER; note = 2'-O-methyl nucleotide
  modified_base 10: mod_base = OTHER; note = 2'-O-methyl nucleotide
  modified_base 11: mod_base = OTHER; note = 2'-O-methyl nucleotide
  modified_base 12: mod_base = OTHER; note = 2'-O-methyl nucleotide
  modified_base 13: mod_base = OTHER; note = 2'-O-methyl nucleotide
  modified_base 14: mod_base = OTHER; note = 2'-Fluoro nucleotide
  modified_base 15: mod_base = OTHER; note = 2'-O-methyl nucleotide
  modified_base 16: mod_base = OTHER; note = 2'-O-methyl nucleotide
  modified_base 17: mod_base = OTHER; note = 2'-O-methyl nucleotide
  modified_base 18: mod_base = OTHER; note = 2'-O-methyl nucleotide
  modified_base 19: mod_base = OTHER; note = 2'-O-methyl nucleotide phosphorothioate
  modified_base 20: mod_base = OTHER; note = 2'-O-methyl nucleotide phosphorothioate
  modified_base 21: mod_base = OTHER; note = 2'-O-methyl nucleotide
tatcgactccttctatggttt

SEQ ID NO: 242 - RNA, 19 nt, synthetic construct
  source 1..19: mol_type = other RNA
  modified_base 1: mod_base = OTHER; note = 2'-O-methyl nucleotide phosphorothioate
  modified_base 2: mod_base = OTHER; note = 2'-O-methyl nucleotide phosphorothioate
  modified_base 3: mod_base = OTHER; note = 2'-Fluoro nucleotide
  modified_base 4: mod_base = OTHER; note = 2'-O-methyl nucleotide
  modified_base 5: mod_base = OTHER; note = 2'-O-methyl nucleotide
  modified_base 6: mod_base = OTHER; note = 2'-O-methyl nucleotide
  modified_base 7: mod_base = OTHER; note = 2'-Fluoro nucleotide
  modified_base 8: mod_base = OTHER; note = 2'-Fluoro nucleotide
  modified_base 9: mod_base = OTHER; note = 2'-Fluoro nucleotide
  modified_base 10: mod_base = OTHER; note = 2'-O-methyl nucleotide
  modified_base 11: mod_base = OTHER; note = 2'-O-methyl nucleotide
  modified_base 12: mod_base = OTHER; note = 2'-Fluoro nucleotide
  modified_base 13: mod_base = OTHER; note = 2'-O-methyl nucleotide
  modified_base 14: mod_base = OTHER; note = 2'-O-methyl nucleotide
  modified_base 15: mod_base = OTHER; note = 2'-O-methyl nucleotide
  modified_base 16: mod_base = OTHER; note = 2'-O-methyl nucleotide
  modified_base 17: mod_base = OTHER; note = 2'-Fluoro nucleotide
  modified_base 18: mod_base = OTHER; note = 2'-O-methyl nucleotide
  modified_base 19: mod_base = OTHER; note = 2'-O-methyl nucleotide
ggattatctggatgtctaa

SEQ ID NO: 243 - RNA, 21 nt, synthetic construct
  source 1..21: mol_type = other RNA
  modified_base 1: mod_base = OTHER; note = 2'-O-methyl nucleotide phosphorothioate
  modified_base 2: mod_base = OTHER; note = 2'-Fluoro nucleotide phosphorothioate
  modified_base 3: mod_base = OTHER; note = 2'-O-methyl nucleotide
  modified_base 4: mod_base = OTHER; note = 2'-O-methyl nucleotide
  modified_base 5: mod_base = OTHER; note = 2'-O-methyl nucleotide
  modified_base 6: mod_base = OTHER; note = 2'-O-methyl nucleotide
  modified_base 7: mod_base = OTHER; note = 2'-O-methyl nucleotide
  modified_base 8: mod_base = OTHER; note = 2'-O-methyl nucleotide
  modified_base 9: mod_base = OTHER; note = 2'-O-methyl nucleotide
  modified_base 10: mod_base = OTHER; note = 2'-O-methyl nucleotide
  modified_base 11: mod_base = OTHER; note = 2'-O-methyl nucleotide
  modified_base 12: mod_base = OTHER; note = 2'-O-methyl nucleotide
  modified_base 13: mod_base = OTHER; note = 2'-O-methyl nucleotide
  modified_base 14: mod_base = OTHER; note = 2'-Fluoro nucleotide
  modified_base 15: mod_base = OTHER; note = 2'-O-methyl nucleotide
  modified_base 16: mod_base = OTHER; note = 2'-O-methyl nucleotide
  modified_base 17: mod_base = OTHER; note = 2'-O-methyl nucleotide
  modified_base 18: mod_base = OTHER; note = 2'-O-methyl nucleotide
  modified_base 19: mod_base = OTHER; note = 2'-O-methyl nucleotide phosphorothioate
  modified_base 20: mod_base = OTHER; note = 2'-O-methyl nucleotide phosphorothioate
  modified_base 21: mod_base = OTHER; note = 2'-O-methyl nucleotide
ttagacatccagataatcctt

SEQ ID NO: 244 - RNA, 19 nt, synthetic construct
  source 1..19: mol_type = other RNA
  modified_base 1: mod_base = OTHER; note = 2'-O-methyl nucleotide phosphorothioate
  modified_base 2: mod_base = OTHER; note = 2'-O-methyl nucleotide phosphorothioate
  modified_base 3: mod_base = OTHER; note = 2'-Fluoro nucleotide
  modified_base 4: mod_base = OTHER; note = 2'-O-methyl nucleotide
  modified_base 5: mod_base = OTHER; note = 2'-O-methyl nucleotide
  modified_base 6: mod_base = OTHER; note = 2'-O-methyl nucleotide
  modified_base 7: mod_base = OTHER; note = 2'-Fluoro nucleotide
  modified_base 8: mod_base = OTHER; note = 2'-Fluoro nucleotide
  modified_base 9: mod_base = OTHER; note = 2'-Fluoro nucleotide
  modified_base 10: mod_base = OTHER; note = 2'-O-methyl nucleotide
  modified_base 11: mod_base = OTHER; note = 2'-O-methyl nucleotide
  modified_base 12: mod_base = OTHER; note = 2'-Fluoro nucleotide
  modified_base 13: mod_base = OTHER; note = 2'-O-methyl nucleotide
  modified_base 14: mod_base = OTHER; note = 2'-O-methyl nucleotide
  modified_base 15: mod_base = OTHER; note = 2'-O-methyl nucleotide
  modified_base 16: mod_base = OTHER; note = 2'-O-methyl nucleotide
  modified_base 17: mod_base = OTHER; note = 2'-Fluoro nucleotide
  modified_base 18: mod_base = OTHER; note = 2'-O-methyl nucleotide
  modified_base 19: mod_base = OTHER; note = 2'-O-methyl nucleotide
acgttgccctgatcaagca

SEQ ID NO: 245 - RNA, 21 nt, synthetic construct
  source 1..21: mol_type = other RNA
  modified_base 1: mod_base = OTHER; note = 2'-O-methyl nucleotide phosphorothioate
  modified_base 2: mod_base = OTHER; note = 2'-Fluoro nucleotide phosphorothioate
  modified_base 3: mod_base = OTHER; note = 2'-O-methyl nucleotide
  modified_base 4: mod_base = OTHER; note = 2'-O-methyl nucleotide
  modified_base 5: mod_base = OTHER; note = 2'-O-methyl nucleotide
  modified_base 6: mod_base = OTHER; note = 2'-O-methyl nucleotide
  modified_base 7: mod_base = OTHER; note = 2'-O-methyl nucleotide
  modified_base 8: mod_base = OTHER; note = 2'-O-methyl nucleotide
  modified_base 9: mod_base = OTHER; note = 2'-O-methyl nucleotide
  modified_base 10: mod_base = OTHER; note = 2'-O-methyl nucleotide
  modified_base 11: mod_base = OTHER; note = 2'-O-methyl nucleotide
  modified_base 12: mod_base = OTHER; note = 2'-O-methyl nucleotide
  modified_base 13: mod_base = OTHER; note = 2'-O-methyl nucleotide
  modified_base 14: mod_base = OTHER; note = 2'-Fluoro nucleotide
  modified_base 15: mod_base = OTHER; note = 2'-O-methyl nucleotide
  modified_base 16: mod_base = OTHER; note = 2'-O-methyl nucleotide
  modified_base 17: mod_base = OTHER; note = 2'-O-methyl nucleotide
  modified_base 18: mod_base = OTHER; note = 2'-O-methyl nucleotide
  modified_base 19: mod_base = OTHER; note = 2'-O-methyl nucleotide phosphorothioate
  modified_base 20: mod_base = OTHER; note = 2'-O-methyl nucleotide phosphorothioate
  modified_base 21: mod_base = OTHER; note = 2'-O-methyl nucleotide
tgcttgatcagggcaacgttt

SEQ ID NO: 246 - RNA, 19 nt, synthetic construct
  source 1..19: mol_type = other RNA
  modified_base 1: mod_base = OTHER; note = 2'-O-methyl nucleotide phosphorothioate
  modified_base 2: mod_base = OTHER; note = 2'-O-methyl nucleotide phosphorothioate
  modified_base 3: mod_base = OTHER; note = 2'-Fluoro nucleotide
  modified_base 4: mod_base = OTHER; note = 2'-O-methyl nucleotide
  modified_base 5: mod_base = OTHER; note = 2'-O-methyl nucleotide
  modified_base 6: mod_base = OTHER; note = 2'-O-methyl nucleotide
  modified_base 7: mod_base = OTHER; note = 2'-Fluoro nucleotide
  modified_base 8: mod_base = OTHER; note = 2'-Fluoro nucleotide
  modified_base 9: mod_base = OTHER; note = 2'-Fluoro nucleotide
  modified_base 10: mod_base = OTHER; note = 2'-O-methyl nucleotide
  modified_base 11: mod_base = OTHER; note = 2'-O-methyl nucleotide
  modified_base 12: mod_base = OTHER; note = 2'-Fluoro nucleotide
  modified_base 13: mod_base = OTHER; note = 2'-O-methyl nucleotide
  modified_base 14: mod_base = OTHER; note = 2'-O-methyl nucleotide
  modified_base 15: mod_base = OTHER; note = 2'-O-methyl nucleotide
  modified_base 16: mod_base = OTHER; note = 2'-O-methyl nucleotide
  modified_base 17: mod_base = OTHER; note = 2'-Fluoro nucleotide
  modified_base 18: mod_base = OTHER; note = 2'-O-methyl nucleotide
  modified_base 19: mod_base = OTHER; note = 2'-O-methyl nucleotide
ggagccaaaaagtgtctaa

SEQ ID NO: 247 - RNA, 21 nt, synthetic construct
  source 1..21: mol_type = other RNA
  modified_base 1: mod_base = OTHER; note = 2'-O-methyl nucleotide phosphorothioate
  modified_base 2: mod_base = OTHER; note = 2'-Fluoro nucleotide phosphorothioate
  modified_base 3: mod_base = OTHER; note = 2'-O-methyl nucleotide
  modified_base 4: mod_base = OTHER; note = 2'-O-methyl nucleotide
  modified_base 5: mod_base = OTHER; note = 2'-O-methyl nucleotide
  modified_base 6: mod_base = OTHER; note = 2'-O-methyl nucleotide
  modified_base 7: mod_base = OTHER; note = 2'-O-methyl nucleotide
  modified_base 8: mod_base = OTHER; note = 2'-O-methyl nucleotide
  modified_base 9: mod_base = OTHER; note = 2'-O-methyl nucleotide
  modified_base 10: mod_base = OTHER; note = 2'-O-methyl nucleotide
  modified_base 11: mod_base = OTHER; note = 2'-O-methyl nucleotide
  modified_base 12: mod_base = OTHER; note = 2'-O-methyl nucleotide
  modified_base 13: mod_base = OTHER; note = 2'-O-methyl nucleotide
  modified_base 14: mod_base = OTHER; note = 2'-Fluoro nucleotide
  modified_base 15: mod_base = OTHER; note = 2'-O-methyl nucleotide
  modified_base 16: mod_base = OTHER; note = 2'-O-methyl nucleotide
  modified_base 17: mod_base = OTHER; note = 2'-O-methyl nucleotide
  modified_base 18: mod_base = OTHER; note = 2'-O-methyl nucleotide
  modified_base 19: mod_base = OTHER; note = 2'-O-methyl nucleotide phosphorothioate
  modified_base 20: mod_base = OTHER; note = 2'-O-methyl nucleotide phosphorothioate
  modified_base 21: mod_base = OTHER; note = 2'-O-methyl nucleotide
ttagacactttttggctcctt

SEQ ID NO: 248 - RNA, 19 nt, synthetic construct
  source 1..19: mol_type = other RNA
  modified_base 1: mod_base = OTHER; note = 2'-O-methyl nucleotide phosphorothioate
  modified_base 2: mod_base = OTHER; note = 2'-O-methyl nucleotide phosphorothioate
  modified_base 3: mod_base = OTHER; note = 2'-Fluoro nucleotide
  modified_base 4: mod_base = OTHER; note = 2'-O-methyl nucleotide
  modified_base 5: mod_base = OTHER; note = 2'-O-methyl nucleotide
  modified_base 6: mod_base = OTHER; note = 2'-O-methyl nucleotide
  modified_base 7: mod_base = OTHER; note = 2'-Fluoro nucleotide
  modified_base 8: mod_base = OTHER; note = 2'-Fluoro nucleotide
  modified_base 9: mod_base = OTHER; note = 2'-Fluoro nucleotide
  modified_base 10: mod_base = OTHER; note = 2'-O-methyl nucleotide
  modified_base 11: mod_base = OTHER; note = 2'-O-methyl nucleotide
  modified_base 12: mod_base = OTHER; note = 2'-Fluoro nucleotide
  modified_base 13: mod_base = OTHER; note = 2'-O-methyl nucleotide
  modified_base 14: mod_base = OTHER; note = 2'-O-methyl nucleotide
  modified_base 15: mod_base = OTHER; note = 2'-O-methyl nucleotide
  modified_base 16: mod_base = OTHER; note = 2'-O-methyl nucleotide
  modified_base 17: mod_base = OTHER; note = 2'-Fluoro nucleotide
  modified_base 18: mod_base = OTHER; note = 2'-O-methyl nucleotide
  modified_base 19: mod_base = OTHER; note = 2'-O-methyl nucleotide
ccgattaccacaagcaaca

SEQ ID NO: 249 - RNA, 21 nt, synthetic construct
  source 1..21: mol_type = other RNA
  modified_base 1: mod_base = OTHER; note = 2'-O-methyl nucleotide phosphorothioate
  modified_base 2: mod_base = OTHER; note = 2'-Fluoro nucleotide phosphorothioate
  modified_base 3: mod_base = OTHER; note = 2'-O-methyl nucleotide
  modified_base 4: mod_base = OTHER; note = 2'-O-methyl nucleotide
  modified_base 5: mod_base = OTHER; note = 2'-O-methyl nucleotide
  modified_base 6: mod_base = OTHER; note = 2'-O-methyl nucleotide
  modified_base 7: mod_base = OTHER; note = 2'-O-methyl nucleotide
  modified_base 8: mod_base = OTHER; note = 2'-O-methyl nucleotide
  modified_base 9: mod_base = OTHER; note = 2'-O-methyl nucleotide
  modified_base 10: mod_base = OTHER; note = 2'-O-methyl nucleotide
  modified_base 11: mod_base = OTHER; note = 2'-O-methyl nucleotide
  modified_base 12: mod_base = OTHER; note = 2'-O-methyl nucleotide
  modified_base 13: mod_base = OTHER; note = 2'-O-methyl nucleotide
  modified_base 14: mod_base = OTHER; note = 2'-Fluoro nucleotide
  modified_base 15: mod_base = OTHER; note = 2'-O-methyl nucleotide
  modified_base 16: mod_base = OTHER; note = 2'-O-methyl nucleotide
  modified_base 17: mod_base = OTHER; note = 2'-O-methyl nucleotide
  modified_base 18: mod_base = OTHER; note = 2'-O-methyl nucleotide
  modified_base 19: mod_base = OTHER; note = 2'-O-methyl nucleotide phosphorothioate
  modified_base 20: mod_base = OTHER; note = 2'-O-methyl nucleotide phosphorothioate
  modified_base 21: mod_base = OTHER; note = 2'-O-methyl nucleotide
tgttgcttgtggtaatcggtt

SEQ ID NO: 250 - RNA, 19 nt, synthetic construct
  source 1..19: mol_type = other RNA
  modified_base 1: mod_base = OTHER; note = 2'-O-methyl nucleotide phosphorothioate
  modified_base 2: mod_base = OTHER; note = 2'-O-methyl nucleotide phosphorothioate
  modified_base 3: mod_base = OTHER; note = 2'-Fluoro nucleotide
  modified_base 4: mod_base = OTHER; note = 2'-O-methyl nucleotide
  modified_base 5: mod_base = OTHER; note = 2'-O-methyl nucleotide
  modified_base 6: mod_base = OTHER; note = 2'-O-methyl nucleotide
  modified_base 7: mod_base = OTHER; note = 2'-Fluoro nucleotide
  modified_base 8: mod_base = OTHER; note = 2'-Fluoro nucleotide
  modified_base 9: mod_base = OTHER; note = 2'-Fluoro nucleotide
  modified_base 10: mod_base = OTHER; note = 2'-O-methyl nucleotide
  modified_base 11: mod_base = OTHER; note = 2'-O-methyl nucleotide
  modified_base 12: mod_base = OTHER; note = 2'-Fluoro nucleotide
  modified_base 13: mod_base = OTHER; note = 2'-O-methyl nucleotide
  modified_base 14: mod_base = OTHER; note = 2'-O-methyl nucleotide
  modified_base 15: mod_base = OTHER; note = 2'-O-methyl nucleotide
  modified_base 16: mod_base = OTHER; note = 2'-O-methyl nucleotide
  modified_base 17: mod_base = OTHER; note = 2'-Fluoro nucleotide
  modified_base 18: mod_base = OTHER; note = 2'-O-methyl nucleotide
  modified_base 19: mod_base = OTHER; note = 2'-O-methyl nucleotide
aatgcagactgggtcacga

SEQ ID NO: 251 - RNA, 21 nt, synthetic construct
  source 1..21: mol_type = other RNA
  modified_base 1: mod_base = OTHER; note = 2'-O-methyl nucleotide phosphorothioate
  modified_base 2: mod_base = OTHER; note = 2'-Fluoro nucleotide phosphorothioate
  modified_base 3: mod_base = OTHER; note = 2'-O-methyl nucleotide
  modified_base 4: mod_base = OTHER; note = 2'-O-methyl nucleotide
  modified_base 5: mod_base = OTHER; note = 2'-O-methyl nucleotide
  modified_base 6: mod_base = OTHER; note = 2'-O-methyl nucleotide
  modified_base 7: mod_base = OTHER; note = 2'-O-methyl nucleotide
  modified_base 8: mod_base = OTHER; note = 2'-O-methyl nucleotide
  modified_base 9: mod_base = OTHER; note = 2'-O-methyl nucleotide
  modified_base 10: mod_base = OTHER; note = 2'-O-methyl nucleotide
  modified_base 11: mod_base = OTHER; note = 2'-O-methyl nucleotide
  modified_base 12: mod_base = OTHER; note = 2'-O-methyl nucleotide
  modified_base 13: mod_base = OTHER; note = 2'-O-methyl nucleotide
  modified_base 14: mod_base = OTHER; note = 2'-Fluoro nucleotide
  modified_base 15: mod_base = OTHER; note = 2'-O-methyl nucleotide
  modified_base 16: mod_base = OTHER; note = 2'-O-methyl nucleotide
  modified_base 17: mod_base = OTHER; note = 2'-O-methyl nucleotide
  modified_base 18: mod_base = OTHER; note = 2'-O-methyl nucleotide
  modified_base 19: mod_base = OTHER; note = 2'-O-methyl nucleotide phosphorothioate
  modified_base 20: mod_base = OTHER; note = 2'-O-methyl nucleotide phosphorothioate
  modified_base 21: mod_base = OTHER; note = 2'-O-methyl nucleotide
tcgtgacccagtctgcatttt

SEQ ID NO: 252 - RNA, 19 nt, synthetic construct
  source 1..19: mol_type = other RNA
  modified_base 1: mod_base = OTHER; note = 2'-O-methyl nucleotide phosphorothioate
  modified_base 2: mod_base = OTHER; note = 2'-O-methyl nucleotide phosphorothioate
  modified_base 3: mod_base = OTHER; note = 2'-Fluoro nucleotide
  modified_base 4: mod_base = OTHER; note = 2'-O-methyl nucleotide
  modified_base 5: mod_base = OTHER; note = 2'-O-methyl nucleotide
  modified_base 6: mod_base = OTHER; note = 2'-O-methyl nucleotide
  modified_base 7: mod_base = OTHER; note = 2'-Fluoro nucleotide
  modified_base 8: mod_base = OTHER; note = 2'-Fluoro nucleotide
  modified_base 9: mod_base = OTHER; note = 2'-Fluoro nucleotide
  modified_base 10: mod_base = OTHER; note = 2'-O-methyl nucleotide
  modified_base 11: mod_base = OTHER; note = 2'-O-methyl nucleotide
  modified_base 12: mod_base = OTHER; note = 2'-Fluoro nucleotide
  modified_base 13: mod_base = OTHER; note = 2'-O-methyl nucleotide
  modified_base 14: mod_base = OTHER; note = 2'-O-methyl nucleotide
  modified_base 15: mod_base = OTHER; note = 2'-O-methyl nucleotide
  modified_base 16: mod_base = OTHER; note = 2'-O-methyl nucleotide
  modified_base 17: mod_base = OTHER; note = 2'-Fluoro nucleotide
  modified_base 18: mod_base = OTHER; note = 2'-O-methyl nucleotide
  modified_base 19: mod_base = OTHER; note = 2'-O-methyl nucleotide
ctcacgcccgagactttca

SEQ ID NO: 253 - RNA, 21 nt, synthetic construct
  source 1..21: mol_type = other RNA
  modified_base 1: mod_base = OTHER; note = 2'-O-methyl nucleotide phosphorothioate
  modified_base 2: mod_base = OTHER; note = 2'-Fluoro nucleotide phosphorothioate
  modified_base 3: mod_base = OTHER; note = 2'-O-methyl nucleotide
  modified_base 4: mod_base = OTHER; note = 2'-O-methyl nucleotide
  modified_base 5: mod_base = OTHER; note = 2'-O-methyl nucleotide
  modified_base 6: mod_base = OTHER; note = 2'-O-methyl nucleotide
  modified_base 7: mod_base = OTHER; note = 2'-O-methyl nucleotide
  modified_base 8: mod_base = OTHER; note = 2'-O-methyl nucleotide
  modified_base 9: mod_base = OTHER; note = 2'-O-methyl nucleotide
  modified_base 10: mod_base = OTHER; note = 2'-O-methyl nucleotide
  modified_base 11: mod_base = OTHER; note = 2'-O-methyl nucleotide
  modified_base 12: mod_base = OTHER; note = 2'-O-methyl nucleotide
  modified_base 13: mod_base = OTHER; note = 2'-O-methyl nucleotide
  modified_base 14: mod_base = OTHER; note = 2'-Fluoro nucleotide
  modified_base 15: mod_base = OTHER; note = 2'-O-methyl nucleotide
  modified_base 16: mod_base = OTHER; note = 2'-O-methyl nucleotide
  modified_base 17: mod_base = OTHER; note = 2'-O-methyl nucleotide
  modified_base 18: mod_base = OTHER; note = 2'-O-methyl nucleotide
  modified_base 19: mod_base = OTHER; note = 2'-O-methyl nucleotide phosphorothioate
  modified_base 20: mod_base = OTHER; note = 2'-O-methyl nucleotide phosphorothioate
  modified_base 21: mod_base = OTHER; note = 2'-O-methyl nucleotide
tgaaagtctcgggcgtgagtt

SEQ ID NO: 254 - RNA, 19 nt, synthetic construct
  source 1..19: mol_type = other RNA
  modified_base 1: mod_base = OTHER; note = 2'-O-methyl nucleotide phosphorothioate
  modified_base 2: mod_base = OTHER; note = 2'-O-methyl nucleotide phosphorothioate
  modified_base 3: mod_base = OTHER; note = 2'-Fluoro nucleotide
  modified_base 4: mod_base = OTHER; note = 2'-O-methyl nucleotide
  modified_base 5: mod_base = OTHER; note = 2'-O-methyl nucleotide
  modified_base 6: mod_base = OTHER; note = 2'-O-methyl nucleotide
  modified_base 7: mod_base = OTHER; note = 2'-Fluoro nucleotide
  modified_base 8: mod_base = OTHER; note = 2'-Fluoro nucleotide
  modified_base 9: mod_base = OTHER; note = 2'-Fluoro nucleotide
  modified_base 10: mod_base = OTHER; note = 2'-O-methyl nucleotide
  modified_base 11: mod_base = OTHER; note = 2'-O-methyl nucleotide
  modified_base 12: mod_base = OTHER; note = 2'-Fluoro nucleotide
  modified_base 13: mod_base = OTHER; note = 2'-O-methyl nucleotide
  modified_base 14: mod_base = OTHER; note = 2'-O-methyl nucleotide
  modified_base 15: mod_base = OTHER; note = 2'-O-methyl nucleotide
  modified_base 16: mod_base = OTHER; note = 2'-O-methyl nucleotide
  modified_base 17: mod_base = OTHER; note = 2'-Fluoro nucleotide
  modified_base 18: mod_base = OTHER; note = 2'-O-methyl nucleotide
  modified_base 19: mod_base = OTHER; note = 2'-O-methyl nucleotide
cagcgatctgtgacaacga

SEQ ID NO: 255 - RNA, 21 nt, synthetic construct
  source 1..21: mol_type = other RNA
  modified_base 1: mod_base = OTHER; note = 2'-O-methyl nucleotide phosphorothioate
  modified_base 2: mod_base = OTHER; note = 2'-Fluoro nucleotide phosphorothioate
  modified_base 3: mod_base = OTHER; note = 2'-O-methyl nucleotide
  modified_base 4: mod_base = OTHER; note = 2'-O-methyl nucleotide
  modified_base 5: mod_base = OTHER; note = 2'-O-methyl nucleotide
  modified_base 6: mod_base = OTHER; note = 2'-O-methyl nucleotide
  modified_base 7: mod_base = OTHER; note = 2'-O-methyl nucleotide
  modified_base 8: mod_base = OTHER; note = 2'-O-methyl nucleotide
  modified_base 9: mod_base = OTHER; note = 2'-O-methyl nucleotide
  modified_base 10: mod_base = OTHER; note = 2'-O-methyl nucleotide
  modified_base 11: mod_base = OTHER; note = 2'-O-methyl nucleotide
  modified_base 12: mod_base = OTHER; note = 2'-O-methyl nucleotide
  modified_base 13: mod_base = OTHER; note = 2'-O-methyl nucleotide
  modified_base 14: mod_base = OTHER; note = 2'-Fluoro nucleotide
  modified_base 15: mod_base = OTHER; note = 2'-O-methyl nucleotide
  modified_base 16: mod_base = OTHER; note = 2'-O-methyl nucleotide
  modified_base 17: mod_base = OTHER; note = 2'-O-methyl nucleotide
  modified_base 18: mod_base = OTHER; note = 2'-O-methyl nucleotide
  modified_base 19: mod_base = OTHER; note = 2'-O-methyl nucleotide phosphorothioate
  modified_base 20: mod_base = OTHER; note = 2'-O-methyl nucleotide phosphorothioate
  modified_base 21: mod_base = OTHER; note = 2'-O-methyl nucleotide
tcgttgtcacagatcgctgtt

SEQ ID NO: 256 - RNA, 19 nt, synthetic construct
  source 1..19: mol_type = other RNA
  modified_base 1: mod_base = OTHER; note = 2'-O-methyl nucleotide phosphorothioate
  modified_base 2: mod_base = OTHER; note = 2'-O-methyl nucleotide phosphorothioate
  modified_base 3: mod_base = OTHER; note = 2'-Fluoro nucleotide
  modified_base 4: mod_base = OTHER; note = 2'-O-methyl nucleotide
  modified_base 5: mod_base = OTHER; note = 2'-O-methyl nucleotide
  modified_base 6: mod_base = OTHER; note = 2'-O-methyl nucleotide
  modified_base 7: mod_base = OTHER; note = 2'-Fluoro nucleotide
  modified_base 8: mod_base = OTHER; note = 2'-Fluoro nucleotide
  modified_base 9: mod_base = OTHER; note = 2'-Fluoro nucleotide
  modified_base 10: mod_base = OTHER; note = 2'-O-methyl nucleotide
  modified_base 11: mod_base = OTHER; note = 2'-O-methyl nucleotide
  modified_base 12: mod_base = OTHER; note = 2'-Fluoro nucleotide
  modified_base 13: mod_base = OTHER; note = 2'-O-methyl nucleotide
  modified_base 14: mod_base = OTHER; note = 2'-O-methyl nucleotide
  modified_base 15: mod_base = OTHER; note = 2'-O-methyl nucleotide
  modified_base 16: mod_base = OTHER; note = 2'-O-methyl nucleotide
  modified_base 17: mod_base = OTHER; note = 2'-Fluoro nucleotide
  modified_base 18: mod_base = OTHER; note = 2'-O-methyl nucleotide
  modified_base 19: mod_base = OTHER; note = 2'-O-methyl nucleotide
ctaccacttgccagcaaca

SEQ ID NO: 257 - RNA, 21 nt, synthetic construct
  source 1..21: mol_type = other RNA
  modified_base 1: mod_base = OTHER; note = 2'-O-methyl nucleotide phosphorothioate
  modified_base 2: mod_base = OTHER; note = 2'-Fluoro nucleotide phosphorothioate
  modified_base 3: mod_base = OTHER; note = 2'-O-methyl nucleotide
  modified_base 4: mod_base = OTHER; note = 2'-O-methyl nucleotide
  modified_base 5: mod_base = OTHER; note = 2'-O-methyl nucleotide
  modified_base 6: mod_base = OTHER; note = 2'-O-methyl nucleotide
  modified_base 7: mod_base = OTHER; note = 2'-O-methyl nucleotide
  modified_base 8: mod_base = OTHER; note = 2'-O-methyl nucleotide
  modified_base 9: mod_base = OTHER; note = 2'-O-methyl nucleotide
  modified_base 10: mod_base = OTHER; note = 2'-O-methyl nucleotide
  modified_base 11: mod_base = OTHER; note = 2'-O-methyl nucleotide
  modified_base 12: mod_base = OTHER; note = 2'-O-methyl nucleotide
  modified_base 13: mod_base = OTHER; note = 2'-O-methyl nucleotide
  modified_base 14: mod_base = OTHER; note = 2'-Fluoro nucleotide
  modified_base 15: mod_base = OTHER; note = 2'-O-methyl nucleotide
  modified_base 16: mod_base = OTHER; note = 2'-O-methyl nucleotide
  modified_base 17: mod_base = OTHER; note = 2'-O-methyl nucleotide
  modified_base 18: mod_base = OTHER; note = 2'-O-methyl nucleotide
  modified_base 19: mod_base = OTHER; note = 2'-O-methyl nucleotide phosphorothioate
  modified_base 20: mod_base = OTHER; note = 2'-O-methyl nucleotide phosphorothioate
  modified_base 21: mod_base = OTHER; note = 2'-O-methyl nucleotide
tgttgctggcaagtggtagtt

SEQ ID NO: 258 - RNA, 19 nt, synthetic construct
  source 1..19: mol_type = other RNA
  modified_base 1: mod_base = OTHER; note = 2'-O-methyl nucleotide phosphorothioate
  modified_base 2: mod_base = OTHER; note = 2'-O-methyl nucleotide phosphorothioate
  modified_base 3: mod_base = OTHER; note = 2'-Fluoro nucleotide
  modified_base 4: mod_base = OTHER; note = 2'-O-methyl nucleotide
  modified_base 5: mod_base = OTHER; note = 2'-O-methyl nucleotide
  modified_base 6: mod_base = OTHER; note = 2'-O-methyl nucleotide
  modified_base 7: mod_base = OTHER; note = 2'-Fluoro nucleotide
  modified_base 8: mod_base = OTHER; note = 2'-Fluoro nucleotide
  modified_base 9: mod_base = OTHER; note = 2'-Fluoro nucleotide
  modified_base 10: mod_base = OTHER; note = 2'-O-methyl nucleotide
  modified_base 11: mod_base = OTHER; note = 2'-O-methyl nucleotide
  modified_base 12: mod_base = OTHER; note = 2'-Fluoro nucleotide
  modified_base 13: mod_base = OTHER; note = 2'-O-methyl nucleotide
  modified_base 14: mod_base = OTHER; note = 2'-O-methyl nucleotide
  modified_base 15: mod_base = OTHER; note = 2'-O-methyl nucleotide
  modified_base 16: mod_base = OTHER; note = 2'-O-methyl nucleotide
  modified_base 17: mod_base = OTHER; note = 2'-Fluoro nucleotide
  modified_base 18: mod_base = OTHER; note = 2'-O-methyl nucleotide
  modified_base 19: mod_base = OTHER; note = 2'-O-methyl nucleotide
tttatgactatgacgttga

SEQ ID NO: 259 - RNA, 21 nt, synthetic construct
  source 1..21: mol_type = other RNA
  modified_base 1: mod_base = OTHER; note = 2'-O-methyl nucleotide phosphorothioate
  modified_base 2: mod_base = OTHER; note = 2'-Fluoro nucleotide phosphorothioate
  modified_base 3: mod_base = OTHER; note = 2'-O-methyl nucleotide
  modified_base 4: mod_base = OTHER; note = 2'-O-methyl nucleotide
  modified_base 5: mod_base = OTHER; note = 2'-O-methyl nucleotide
  modified_base 6: mod_base = OTHER; note = 2'-O-methyl nucleotide
  modified_base 7: mod_base = OTHER; note = 2'-O-methyl nucleotide
  modified_base 8: mod_base = OTHER; note = 2'-O-methyl nucleotide
  modified_base 9: mod_base = OTHER; note = 2'-O-methyl nucleotide
  modified_base 10: mod_base = OTHER; note = 2'-O-methyl nucleotide
  modified_base 11: mod_base = OTHER; note = 2'-O-methyl nucleotide
  modified_base 12: mod_base = OTHER; note = 2'-O-methyl nucleotide
  modified_base 13: mod_base = OTHER; note = 2'-O-methyl nucleotide
  modified_base 14: mod_base = OTHER; note = 2'-Fluoro nucleotide
  modified_base 15: mod_base = OTHER; note = 2'-O-methyl nucleotide
  modified_base 16: mod_base = OTHER; note = 2'-O-methyl nucleotide
  modified_base 17: mod_base = OTHER; note = 2'-O-methyl nucleotide
  modified_base 18: mod_base = OTHER; note = 2'-O-methyl nucleotide
  modified_base 19: mod_base = OTHER; note = 2'-O-methyl nucleotide phosphorothioate
  modified_base 20: mod_base = OTHER; note = 2'-O-methyl nucleotide phosphorothioate
  modified_base 21: mod_base = OTHER; note = 2'-O-methyl nucleotide
tcaacgtcatagtcataaatt

SEQ ID NO: 260 - RNA, 19 nt, synthetic construct
  source 1..19: mol_type = other RNA
  modified_base 1: mod_base = OTHER; note = 2'-O-methyl nucleotide phosphorothioate
  modified_base 2: mod_base = OTHER; note = 2'-O-methyl nucleotide phosphorothioate
  modified_base 3: mod_base = OTHER; note = 2'-Fluoro nucleotide
  modified_base 4: mod_base = OTHER; note = 2'-O-methyl nucleotide
  modified_base 5: mod_base = OTHER; note = 2'-O-methyl nucleotide
  modified_base 6: mod_base = OTHER; note = 2'-O-methyl nucleotide
  modified_base 7: mod_base = OTHER; note = 2'-Fluoro nucleotide
  modified_base 8: mod_base = OTHER; note = 2'-Fluoro nucleotide
  modified_base 9: mod_base = OTHER; note = 2'-Fluoro nucleotide
  modified_base 10: mod_base = OTHER; note = 2'-O-methyl nucleotide
  modified_base 11: mod_base = OTHER; note = 2'-O-methyl nucleotide
  modified_base 12: mod_base = OTHER; note = 2'-Fluoro nucleotide
  modified_base 13: mod_base = OTHER; note = 2'-O-methyl nucleotide
  modified_base 14: mod_base = OTHER; note = 2'-O-methyl nucleotide
  modified_base 15: mod_base = OTHER; note = 2'-O-methyl nucleotide
  modified_base 16: mod_base = OTHER; note = 2'-O-methyl nucleotide
  modified_base 17: mod_base = OTHER; note = 2'-Fluoro nucleotide
  modified_base 18: mod_base = OTHER; note = 2'-O-methyl nucleotide
  modified_base 19: mod_base = OTHER; note = 2'-O-methyl nucleotide
ccttgatagttcacaagaa

SEQ ID NO: 261 - RNA, 21 nt, synthetic construct
  source 1..21: mol_type = other RNA
  modified_base 1: mod_base = OTHER; note = 2'-O-methyl nucleotide phosphorothioate
  modified_base 2: mod_base = OTHER; note = 2'-Fluoro nucleotide phosphorothioate
  modified_base 3: mod_base = OTHER; note = 2'-O-methyl nucleotide
  modified_base 4: mod_base = OTHER; note = 2'-O-methyl nucleotide
  modified_base 5: mod_base = OTHER; note = 2'-O-methyl nucleotide
  modified_base 6: mod_base = OTHER; note = 2'-O-methyl nucleotide
  modified_base 7: mod_base = OTHER; note = 2'-O-methyl nucleotide
  modified_base 8: mod_base = OTHER; note = 2'-O-methyl nucleotide
  modified_base 9: mod_base = OTHER; note = 2'-O-methyl nucleotide
  modified_base 10: mod_base = OTHER; note = 2'-O-methyl nucleotide
  modified_base 11: mod_base = OTHER; note = 2'-O-methyl nucleotide
  modified_base 12: mod_base = OTHER; note = 2'-O-methyl nucleotide
  modified_base 13: mod_base = OTHER; note = 2'-O-methyl nucleotide
  modified_base 14: mod_base = OTHER; note = 2'-Fluoro nucleotide
  modified_base 15: mod_base = OTHER; note = 2'-O-methyl nucleotide
  modified_base 16: mod_base = OTHER; note = 2'-O-methyl nucleotide
  modified_base 17: mod_base = OTHER; note = 2'-O-methyl nucleotide
  modified_base 18: mod_base = OTHER; note = 2'-O-methyl nucleotide
  modified_base 19: mod_base = OTHER; note = 2'-O-methyl nucleotide phosphorothioate
  modified_base 20: mod_base = OTHER; note = 2'-O-methyl nucleotide phosphorothioate
  modified_base 21: mod_base = OTHER; note = 2'-O-methyl nucleotide
ttcttgtgaactatcaaggtt

SEQ ID NO: 262 - RNA, 19 nt, synthetic construct
  source 1..19: mol_type = other RNA
  modified_base 1: mod_base = OTHER; note = 2'-O-methyl nucleotide phosphorothioate
  modified_base 2: mod_base = OTHER; note = 2'-O-methyl nucleotide phosphorothioate
  modified_base 3: mod_base = OTHER; note = 2'-Fluoro nucleotide
  modified_base 4: mod_base = OTHER; note = 2'-O-methyl nucleotide
  modified_base 5: mod_base = OTHER; note = 2'-O-methyl nucleotide
  modified_base 6: mod_base = OTHER; note = 2'-O-methyl nucleotide
  modified_base 7: mod_base = OTHER; note = 2'-Fluoro nucleotide
  modified_base 8: mod_base = OTHER; note = 2'-Fluoro nucleotide
  modified_base 9: mod_base = OTHER; note = 2'-Fluoro nucleotide
  modified_base 10: mod_base = OTHER; note = 2'-O-methyl nucleotide
  modified_base 11: mod_base = OTHER; note = 2'-O-methyl nucleotide
  modified_base 12: mod_base = OTHER; note = 2'-Fluoro nucleotide
  modified_base 13: mod_base = OTHER; note = 2'-O-methyl nucleotide
  modified_base 14: mod_base = OTHER; note = 2'-O-methyl nucleotide
  modified_base 15: mod_base = OTHER; note = 2'-O-methyl nucleotide
  modified_base 16: mod_base = OTHER; note = 2'-O-methyl nucleotide
  modified_base 17: mod_base = OTHER; note = 2'-Fluoro nucleotide
  modified_base 18: mod_base = OTHER; note = 2'-O-methyl nucleotide
  modified_base 19: mod_base = OTHER; note = 2'-O-methyl nucleotide
ctcaagaggtggccgaaga

SEQ ID NO: 263 - RNA, 21 nt, synthetic construct
  source 1..21: mol_type = other RNA
  modified_base 1: mod_base = OTHER; note = 2'-O-methyl nucleotide phosphorothioate
  modified_base 2: mod_base = OTHER; note = 2'-Fluoro nucleotide phosphorothioate
  modified_base 3: mod_base = OTHER; note = 2'-O-methyl nucleotide
  modified_base 4: mod_base = OTHER; note = 2'-O-methyl nucleotide
  modified_base 5: mod_base = OTHER; note = 2'-O-methyl nucleotide
  modified_base 6: mod_base = OTHER; note = 2'-O-methyl nucleotide
  modified_base 7: mod_base = OTHER; note = 2'-O-methyl nucleotide
  modified_base 8: mod_base = OTHER; note = 2'-O-methyl nucleotide
  modified_base 9: mod_base = OTHER; note = 2'-O-methyl nucleotide
  modified_base 10: mod_base = OTHER; note = 2'-O-methyl nucleotide
  modified_base 11: mod_base = OTHER; note = 2'-O-methyl nucleotide
  modified_base 12: mod_base = OTHER; note = 2'-O-methyl nucleotide
  modified_base 13: mod_base = OTHER; note = 2'-O-methyl nucleotide
  modified_base 14: mod_base = OTHER; note = 2'-Fluoro nucleotide
  modified_base 15: mod_base = OTHER; note = 2'-O-methyl nucleotide
  modified_base 16: mod_base = OTHER; note = 2'-O-methyl nucleotide
  modified_base 17: mod_base = OTHER; note = 2'-O-methyl nucleotide
  modified_base 18: mod_base = OTHER; note = 2'-O-methyl nucleotide
  modified_base 19: mod_base = OTHER; note = 2'-O-methyl nucleotide phosphorothioate
  modified_base 20: mod_base = OTHER; note = 2'-O-methyl nucleotide phosphorothioate
  modified_base 21: mod_base = OTHER; note = 2'-O-methyl nucleotide
tcttcggccacctcttgagtt

SEQ ID NO: 264 - RNA, 19 nt, synthetic construct
  source 1..19: mol_type = other RNA
  modified_base 1: mod_base = OTHER; note = 2'-O-methyl nucleotide phosphorothioate
  modified_base 2: mod_base = OTHER; note = 2'-O-methyl nucleotide phosphorothioate
  modified_base 3: mod_base = OTHER; note = 2'-Fluoro nucleotide
  modified_base 4: mod_base = OTHER; note = 2'-O-methyl nucleotide
  modified_base 5: mod_base = OTHER; note = 2'-O-methyl nucleotide
  modified_base 6: mod_base = OTHER; note = 2'-O-methyl nucleotide
  modified_base 7: mod_base = OTHER; note = 2'-Fluoro nucleotide
  modified_base 8: mod_base = OTHER; note = 2'-Fluoro nucleotide
  modified_base 9: mod_base = OTHER; note = 2'-Fluoro nucleotide
  modified_base 10: mod_base = OTHER; note = 2'-O-methyl nucleotide
  modified_base 11: mod_base = OTHER; note = 2'-O-methyl nucleotide
  modified_base 12: mod_base = OTHER; note = 2'-Fluoro nucleotide
  modified_base 13: mod_base = OTHER; note = 2'-O-methyl nucleotide
  modified_base 14: mod_base = OTHER; note = 2'-O-methyl nucleotide
  modified_base 15: mod_base = OTHER; note = 2'-O-methyl nucleotide
  modified_base 16: mod_base = OTHER; note = 2'-O-methyl nucleotide
  modified_base 17: mod_base = OTHER; note = 2'-Fluoro nucleotide
  modified_base 18: mod_base = OTHER; note = 2'-O-methyl nucleotide
  modified_base 19: mod_base = OTHER; note = 2'-O-methyl nucleotide
ttttatgactatgacgtta

SEQ ID NO: 265 - RNA, 21 nt, synthetic construct
  source 1..21: mol_type = other RNA
  modified_base 1: mod_base = OTHER; note = 2'-O-methyl nucleotide phosphorothioate
  modified_base 2: mod_base = OTHER; note = 2'-Fluoro nucleotide phosphorothioate
  modified_base 3: mod_base = OTHER; note = 2'-O-methyl nucleotide
  modified_base 4: mod_base = OTHER; note = 2'-O-methyl nucleotide
  modified_base 5: mod_base = OTHER; note = 2'-O-methyl nucleotide
  modified_base 6: mod_base = OTHER; note = 2'-O-methyl nucleotide
  modified_base 7: mod_base = OTHER; note = 2'-O-methyl nucleotide
  modified_base 8: mod_base = OTHER; note = 2'-O-methyl nucleotide
  modified_base 9: mod_base = OTHER; note = 2'-O-methyl nucleotide
  modified_base 10: mod_base = OTHER; note = 2'-O-methyl nucleotide
  modified_base 11: mod_base = OTHER; note = 2'-O-methyl nucleotide
  modified_base 12: mod_base = OTHER; note = 2'-O-methyl nucleotide
  modified_base 13: mod_base = OTHER; note = 2'-O-methyl nucleotide
  modified_base 14: mod_base = OTHER; note = 2'-Fluoro nucleotide
  modified_base 15: mod_base = OTHER; note = 2'-O-methyl nucleotide
  modified_base 16: mod_base = OTHER; note = 2'-O-methyl nucleotide
  modified_base 17: mod_base = OTHER; note = 2'-O-methyl nucleotide
  modified_base 18: mod_base = OTHER; note = 2'-O-methyl nucleotide
  modified_base 19: mod_base = OTHER; note = 2'-O-methyl nucleotide phosphorothioate
  modified_base 20: mod_base = OTHER; note = 2'-O-methyl nucleotide phosphorothioate
  modified_base 21: mod_base = OTHER; note = 2'-O-methyl nucleotide
taacgtcatagtcataaaatt

SEQ ID NO: 266 - RNA, 19 nt, synthetic construct
  source 1..19: mol_type = other RNA
  modified_base 1: mod_base = OTHER; note = 2'-O-methyl nucleotide phosphorothioate
  modified_base 2: mod_base = OTHER; note = 2'-O-methyl nucleotide phosphorothioate
  modified_base 3: mod_base = OTHER; note = 2'-Fluoro nucleotide
  modified_base 4: mod_base = OTHER; note = 2'-O-methyl nucleotide
  modified_base 5: mod_base = OTHER; note = 2'-O-methyl nucleotide
  modified_base 6: mod_base = OTHER; note = 2'-O-methyl nucleotide
  modified_base 7: mod_base = OTHER; note = 2'-Fluoro nucleotide
  modified_base 8: mod_base = OTHER; note = 2'-Fluoro nucleotide
  modified_base 9: mod_base = OTHER; note = 2'-Fluoro nucleotide
  modified_base 10: mod_base = OTHER; note = 2'-O-methyl nucleotide
  modified_base 11: mod_base = OTHER; note = 2'-O-methyl nucleotide
  modified_base 12: mod_base = OTHER; note = 2'-Fluoro nucleotide
  modified_base 13: mod_base = OTHER; note = 2'-O-methyl nucleotide
  modified_base 14: mod_base = OTHER; note = 2'-O-methyl nucleotide
  modified_base 15: mod_base = OTHER; note = 2'-O-methyl nucleotide
  modified_base 16: mod_base = OTHER; note = 2'-O-methyl nucleotide
  modified_base 17: mod_base = OTHER; note = 2'-Fluoro nucleotide
  modified_base 18: mod_base = OTHER; note = 2'-O-methyl nucleotide
  modified_base 19: mod_base = OTHER; note = 2'-O-methyl nucleotide
ttccgacttctccaagaga

SEQ ID NO: 267 - RNA, 21 nt, synthetic construct
  source 1..21: mol_type = other RNA
  modified_base 1: mod_base = OTHER; note = 2'-O-methyl nucleotide phosphorothioate
  modified_base 2: mod_base = OTHER; note = 2'-Fluoro nucleotide phosphorothioate
  modified_base 3: mod_base = OTHER; note = 2'-O-methyl nucleotide
  modified_base 4: mod_base = OTHER; note = 2'-O-methyl nucleotide
  modified_base 5: mod_base = OTHER; note = 2'-O-methyl nucleotide
  modified_base 6: mod_base = OTHER; note = 2'-O-methyl nucleotide
  modified_base 7: mod_base = OTHER; note = 2'-O-methyl nucleotide
  modified_base 8: mod_base = OTHER; note = 2'-O-methyl nucleotide
  modified_base 9: mod_base = OTHER; note = 2'-O-methyl nucleotide
  modified_base 10: mod_base = OTHER; note = 2'-O-methyl nucleotide
  modified_base 11: mod_base = OTHER; note = 2'-O-methyl nucleotide
  modified_base 12: mod_base = OTHER; note = 2'-O-methyl nucleotide
  modified_base 13: mod_base = OTHER; note = 2'-O-methyl nucleotide
  modified_base 14: mod_base = OTHER; note = 2'-Fluoro nucleotide
  modified_base 15: mod_base = OTHER; note = 2'-O-methyl nucleotide
  modified_base 16: mod_base = OTHER; note = 2'-O-methyl nucleotide
  modified_base 17: mod_base = OTHER; note = 2'-O-methyl nucleotide
  modified_base 18: mod_base = OTHER; note = 2'-O-methyl nucleotide
  modified_base 19: mod_base = OTHER; note = 2'-O-methyl nucleotide phosphorothioate
  modified_base 20: mod_base = OTHER; note = 2'-O-methyl nucleotide phosphorothioate
  modified_base 21: mod_base = OTHER; note = 2'-O-methyl nucleotide
tctcttggagaagtcggaatt

SEQ ID NO: 268 - protein, 14 aa, synthetic construct
  SITE 1: note = N-term Ac - acetyl Isoleucine
  SITE 4: note = 1-methyl-tryptophan
  DISULFID 2..12: note = Intrachain disulfide bond
  SITE 14: note = 8-amino-3,6-dioxaoctanoic acid spacer lysine amide (side chain modified with a polyethylene glycol ester linker)
ICVXQDWGAHRCTK